(19) United States
(12) Patent Application Publication
(10) Pub. No.: US 2024/0360429 A1
(43) Pub. Date: Oct. 31, 2024

(54) COMPOSITIONS AND METHODS FOR CONTROLLING ADAPTIVE IMMUNITY IN BACTERIA

(71) Applicant: INTERNATIONAL N&H DENMARK APS, Kgs Lyngby (DK)

(72) Inventors: ANNE M. MILLEN, NEW CENTURY, KS (US); DENNIS ROMERO, NEW CENTURY, KS (US); LAURA SIMDON, NEW CENTURY, KS (US)

(21) Appl. No.: 18/683,081

(22) PCT Filed: Aug. 8, 2022
(86) PCT No.: PCT/EP2022/072214

§ 371 Date: Feb. 12, 2024

Related U.S. Application Data
(60) Provisional application No. 63/232,379, filed on Aug. 12, 2021.

(30) Foreign Application Priority Data
Oct. 27, 2021 (EP) 21204903.5

(51) Int. Cl. C12N 9/22; C12N 15/11; C12N 15/74

(52) U.S. Cl. CPC C12N 9/22; C12N 15/11; C12N 15/746; C12N 2310/20

(57) ABSTRACT

Provided herein are compositions for enabling natural adaptive spacer acquisition in CRISPR-Cas systems incapable of adaptation, e.g., non-adapting lactococcal CRISPR-Cas systems. Also provided are methods for using the compositions and products thereof to produce bacterial strains that are resistant, such as bacteriophage resistant, and/or evolved to have a desirable phenotype. Non-adapting CRISPR-Cas systems adapted according to the methods provided herein, and bacteria containing such adapted non-adapting CRISPR-Cas systems, are further provided. The compositions and methods provided herein allow for the controlled induction of natural spacer acquisition via adaptation in CRISPR-Cas systems that are fundamentally incapable of adaptation without modifying the features underlying the non-adapting behavior of the CRISPR-Cas system.

### FIELD OF THE INVENTION

Provided herein are compositions for enabling natural adaptive spacer acquisition in CRISPR-Cas systems incapable of adaptation, e.g., non-adapting lactococcal CRISPR-Cas systems. Also provided are methods for using the compositions and products thereof to produce bacterial strains that are resistant, such as bacteriophage resistant, and/or evolved to have a desirable phenotype. Non-adapting CRISPR-Cas systems adapted according to the methods provided herein, and bacteria containing such adapted non-adapting CRISPR-Cas systems, are further provided. The compositions and methods provided herein allow for the controlled induction of natural spacer acquisition via adaptation in CRISPR-Cas systems that are fundamentally incapable of adaptation without modifying the features underlying the non-adapting behavior of the CRISPR-Cas system.

### BACKGROUND

CRISPR-Cas (Clustered Regularly Interspaced Short Palindromic Repeats-CRISPR associated proteins) is an adaptive immune system found in bacteria and archaea, which affords organisms protection against invasive nucleic acids. The CRISPR-Cas system includes both Cas proteins and a CRISPR array containing short repeat sequences interspaced by short spacer sequences derived from invading nucleic acids. The spacers contained in the CRISPR array can be used to guard against future infection through processes including maturation, where CRISPR RNAs (crRNA) are generated to guide Cas protein machinery to a target invader nucleic acid, and interference, where the crRNA targeted invader nucleic acid is cleaved and/or degraded by the Cas protein machinery.

Acquisition of spacer sequences by the CRISPR-Cas system generally occurs via adaptation, a process in which new spacers are incorporated into the CRISPR array. Interestingly, it has been found that certain CRISPR-Cas systems, for example CRISPR-Cas systems found in species of milk-adapted Lactococcus, a widely used bacteria in the food industry, are able to perform steps of maturation and interference but lack the ability to adapt. Non-adapting CRISPR-Cas systems are thus unable to acquire immunity against new threats, which can have profound consequences for the organism.

Bacterial cultures are used extensively in the food industry, for example, in the production of fermented food products (e.g., yoghurt, cheese, meat products, bakery products, wine, vegetable products), to protect food products from contaminants (e.g., bacterial, yeast, and mold contaminants), and as probiotics. The ability of bacteria in such cultures to defend against invading nucleic acids, for example bacteriophages, is of utmost importance to the success of the bacterial culture. Bacteria having non-adapting CRISPR-Cas systems may be particularly susceptible to invading nucleic acids, thereby jeopardizing the success of the culture. At industrial scale, culture failure may have major economic impacts, ranging from a reduced quality of the fermented product up to the complete loss of the product.

Thus, there is a need for compositions and methods capable of controlling adaptive immunity in bacteria. The compositions and methods provided herein address such needs.

### SUMMARY OF THE INVENTION

In aspects are provided polynucleotides containing a nucleic acid sequence encoding a Clustered Regularly Interspaced Short Palindromic Repeats (CRISPR)-associated protein 1 (Cas1) polypeptide. In aspects are provided polynucleotides containing a nucleic acid sequence encoding a CRISPR-associated endoribonuclease Cas2 (Cas2) polypeptide. Further provided are vectors containing the polynucleotides provided herein. In aspects are provided bacterial strains containing the polynucleotides or vectors provided herein.

In aspects are provided methods of enabling adaptation in a non-adapting CRISPR-Cas system, the method including introducing into a bacterial strain comprising a non-adapting CRISPR-Cas system polynucleotides and/or vectors described herein. In some embodiments, the methods include exposing bacterial strains introduced with polynucleotides and/or vectors described herein to target nucleic acids.

In aspects are provided methods of producing a bacterial strain resistant to a nucleic acid, the method including exposing a bacterial strain to a target nucleic acid, wherein the bacterial strain contains a non-adapting CRISPR-Cas system and polynucleotides and/or vectors provided herein.

In aspects are provided methods of producing a bacterial strain resistant to a bacteriophage, the method including exposing a bacterial strain to a bacteriophage, wherein the bacterial strain contains a non-adapting CRISPR-Cas system and polynucleotides and/or vectors provided herein.

In aspects are provided methods of producing a bacterial strain having a desirable phenotype, the method including exposing a bacterial strain to one or more stressors or selective pressures dependent on a desirable phenotype, wherein the bacterial strain contains a non-adapting CRISPR-Cas system and polynucleotides and/or vectors provided herein.

In aspects are provided bacterial strains produced according to the methods provided herein.

In aspects are provided adapted non-adapting CRISPR-Cas systems produced according to the methods provided herein. In aspects are provided bacterial strains containing adapted non-adapting CRISPR-Cas systems.

In aspects are provided cell cultures including bacterial strains produced according to the methods provided herein. In aspects are provided food products including bacteria or cell cultures provided herein. In aspects are provided dietary supplements containing bacteria or cell cultures provided herein.

In aspects are provided uses of bacterial strains and cell cultures provided herein for preparing a food product or dietary supplement.

In aspects are provided methods for preparing a food product, the methods including fermenting a substrate with a bacterial strain or a cell culture provided herein.

Each of the aspects and embodiments described herein are capable of being used together, unless excluded either explicitly or clearly from the context of the embodiment or aspect.

### BRIEF DESCRIPTION OF THE FIGURES

FIG. 1 shows a simplified diagram of exemplary lactococcal CRISPR-Cas systems. Block arrows represent genes and direction of transcription; repeats (diamonds), spacers (rectangles), leader (L). (Left) Lactococcus raffinolactis (L. raffinolactis) CRISPR-Cas on the chromosome of L. raffinolactis Lr_19_5. (Center) CRISPR-Cas system resident on plasmid p537CR from Lactococcus cremoris (L. cremoris) DGCC7167, formerly referred to as Lactococcus lactis subsp cremoris. The open arrow section depicted at the 3′ end of cas1 represents missing segment as compared to L. raffinolactis. (Right) pRafCas1Cas2: L. raffinolactis Lr_19_5 cas1-cas2 cloned behind promoter p6 on expression vector pTRK989.

FIGS. 2A-2B show spacer acquisition PCR testing. FIG. 2A shows spacer acquisition detected by PCR (arrow) in exemplary Lactococcus cremoris subsp cremoris (L. cremoris subsp cremoris) DGCC12607 that includes a non-adapting CRISPR-Cas system and was transformed with a vector encoding exemplary Cas1 and Cas2 from L. raffinolactis strain Lr_19_5 (pRafCas1Cas2). FIG. 2B shows a higher molecular weight product (white arrow) in exemplary L. cremoris subsp cremoris DGCC12607 that includes a non-adapting CRISPR-Cas system and was transformed with a vector encoding an exemplary Cas1 and Cas2 from L. raffinolactis strain Lr_19_5 (12607-Cas1Cas2) indicating spacer acquisition in the population, while no acquisition was observed in DGCC12607 transformed with Cas1 alone, Cas2 alone, or in DGCC12607 transformed with an exemplary Cas1 and Cas2 from an exemplary Enterococcus italicus strain (12607-italCas1Cas2).

FIGS. 3A-3D show exemplary non-limiting strategies for producing bacterial strains having adapted non-adapting CRISPR-Cas systems. The legend for all figures is shown in FIG. 3A. FIG. 3A shows the introduction of an exemplary cas1-cas2 encoding vector into a bacterium containing an exemplary non-adapting CRISPR-Cas system, challenging the bacterium with multiple bacteriophages to build a CRISPR array (e.g., adaptation), optionally removing the cas1-cas2 containing vector from the bacterium, and naturally conjugating the adapted non-adapting CRISPR-Cas system into a recipient bacterial strain. FIG. 3B shows a method similar to FIG. 3A, but here, the CRISPR array (or portions thereof) of the adapted non-adapting CRISPR-Cas system built by multiple bacteriophage challenge is amplified and transformed into a recipient strain, e.g., a naturally competent recipient strain, already containing an exemplary non-adapting CRISPR-Cas system where it may insert into the resident non-adapting CRISPR-Cas system via homologous recombination. FIG. 3C shows conjugating an exemplary non-adapting CRISPR-Cas system from a donor bacterium to a recipient strain, introducing an exemplary cas1-cas2 encoding vector into the recipient strain, challenging the starter strain with multiple bacteriophages to build a CRISPR array, and removing (e.g., curing) the cas1-cas2 encoding vector from the recipient strain. FIG. 3D shows construction of an exemplary adapted non-adapting CRISPR-Cas system as described in FIGS. 3A and 3B and subcloning acquired spacers and repeat sequences into an expression vector that can be introduced into a recipient strain, e.g., via natural competence.

### DETAILED DESCRIPTION OF THE INVENTION

CRISPR-Cas systems are adaptive immune systems found in bacteria and archaea. In general, CRISPR-Cas systems are composed of two parts: Clustered Regularly Interspaced Short Palindromic Repeats (CRISPR) arrays and CRISPR-associated (Cas) proteins. CRISPR arrays encode individual spacer sequences separated by conserved repeats. The spacer sequences are derived from invading nucleic acids and can be used to target nucleic acids for cleavage and/or degradation in the event of future invasion. Thus, the spacer sequences present in a CRISPR array can be viewed as an immunological memory of prior infections/attacks which represents the acquired immune profile of the organism. Highly diverse Cas proteins are involved in adaptive immune function, such as degrading invading nucleic acids and facilitating spacer acquisition.

CRISPR-Cas systems generally function in three steps: 1) adaptation, where new spacers are incorporated into the CRISPR array; 2) maturation or processing, during which CRISPR RNAs (crRNA) are generated to guide Cas proteins towards their respective nucleic acid targets; and 3) interference, in which the crRNA targeted invader nucleic acid is cleaved and/or degraded by Cas proteins. However, not all CRISPR-Cas systems are capable of performing each of these functions, a notable example of which is a CRISPR-Cas system found in Lactococcus cremoris subsp cremoris, formerly classified as Lactococcus lactis subsp. cremoris, that appears incapable of adaptation.

L. cremoris subsp cremoris is an industrial strain used in a variety of applications including: fermentation of dairy products, e.g., as a starter culture; protection of food products from contaminants (bacterial, yeast, and mold contaminants), e.g., as a protective culture; and as probiotics, e.g., as a probiotic culture. It has been shown previously that a type III-A CRISPR-Cas system present on a conjugative plasmid in L. cremoris subsp cremoris provides resistance against virulent phages by targeting phage RNA and DNA in a transcription-dependent manner. This CRISPR-Cas system has also been shown to provide resistance when the plasmid it resides on is transferred into a Lactococcus lactis subsp lactis. Despite phage resistance activity, the lactococcal CRISPR-Cas system appears to lack the ability to incorporate new spacers into the CRISPR array via adaptation.

Milk-adapted L. cremoris subsp cremoris strains often harbor genes essential for growth in milk on plasmids. Without being bound by theory, it has been suggested that due to the ancillary activity of CRISPR-Cas against plasmids, a non-adapting CRISPR-Cas system may be favorable for lactococci, allowing them to maintain plasmids encoding beneficial traits.

Bacteriophage infection is a major problem in bacterial cultures used in industrial settings. Bacteriophages employ a variety of mechanisms to attack bacteria, and new types of bacteriophages continue to emerge. Strategies used in industry to minimize bacteriophage infection, and thus failure of a bacterial culture, include the use of: (i) mixed starter cultures; and (ii) the alternate use of strains having different phage susceptibility profiles (strain rotation). However, these strategies are met with their own sets of difficulties. For example, repeated sub-culturing of mixed strain cultures can lead to unpredictable changes in the distribution of individual strains, and eventually undesired strain dominance. This in turn may lead to increased susceptibility to phage attack and risk of fermentation failures. With respect to strain rotation, it is difficult and cumbersome to identify and select a sufficient number of strains having different phage type profiles to provide an efficient and reliable rotation program. In addition, the continuous use of strains requires careful monitoring for new infectious phages and the need to quickly substitute a strain which is infected by the new bacteriophage with a resistant strain. In manufacturing plants, where large quantities of bulk starter cultures are made ahead of time, such a quick response is usually not possible.

Alternative strategies to circumvent bacteriophage infection and bacterial culture failure have included engineering the acquired immune profile of bacteria. For example, CRISPR-Cas systems may be generated with immune profiles that provide resistance to known or suspected invasive nucleic acids, e.g., nucleic acids of bacteriophages. In this way, stocks of bacteria with known resistance can be readily prepared for, e.g., bacterial culture preparation and/or strain rotation.

Strategies for engineering acquired immune profiles have included cloning synthetic spacer sequences into CRISPR-Cas systems or challenge of bacteria with nucleic acids of interest to engage natural CRISPR adaptation. However, the design of synthetic spacers may rely upon empirical rather than natural selection of spacer sequences, which could result in the synthetic spacers behaving sub-optimally in defending against infection. In addition, the presence of cloned foreign DNA prevents the bacteria from achieving food-grade status, which would be unacceptable for bacterial cultures used in food production. Nucleic acid challenge of bacteria to engage natural CRISPR adaptation is also not always possible because some CRISPR-Cas systems, such as milk-adapted lactococcal CRISPR-Cas systems, lack the ability to adapt.

It is therefore clear that new strategies for ensuring the success of bacterial cultures, for example against bacteriophage infection, are required. In particular, these new strategies should produce food-grade organisms useful for the food industry.

As described herein, it was surprisingly found that introduction of cas1 and cas2 genes from Lactococcus raffinolactis into an L. cremoris subsp cremoris bacteria harboring a type-IIIA CRISPR-Cas system incapable of adaptation resulted in the non-adapting CRISPR-Cas system acquiring new spacers when phage-challenged. See, e.g., Example 1. It was also surprisingly found that spacers directed to resident plasmids were present in the CRISPR array of the non-adapting CRISPR-Cas system after introduction of the cas1 and cas2 genes from Lactococcus raffinolactis into the exemplary L. cremoris. Given the location of the spacers in the array, it appeared that adaptation occurred even in the absence of phage challenge.

These remarkable findings demonstrate that adaptation can be enabled in non-adapting CRISPR-Cas systems under controlled experimental conditions, for example using the methods and compositions provided herein. The composition and methods described herein not only enable adaptation in response to phage challenge, thereby producing an adapted non-adapting CRISPR-Cas system with specific, e.g., customized, bacteriophage resistance, but may also enable adaptive spacer acquisition directed to mobile genetic elements (MGEs), e.g., plasmids, integrons, and/or the host genome, which provides additional unique advantages of the compositions and methods provided herein. By way of example, spacer sequences directed against antibiotic resistance MGEs may be acquired to produce an adapted non-adapting CRISPR-Cas system capable of removing undesirable antibiotic resistance.

As another example, enablement of adaptation according to the compositions and methods provided herein may be used to produce adapted non-adapting CRISPR-Cas systems with spacers directed against the host genome, e.g., self-targeting spacer sequences. By placing the bacteria of interest under stresses and/or selective pressures dependent on a desired phenotype, spacers may be acquired that modify genes or genomic elements, e.g., gene segments, promoters, repressors, activators, two-component regulatory systems, resulting in expression of a desirable phenotype.

It should be appreciated that the methods and compositions provided herein allow for the induction of natural adaptation. This is advantageous because it allows the bacteria to naturally select spacer sequences for acquisition. Without being bound by theory, naturally selected spacers, e.g., spacers selected by the internal mechanisms of the bacteria, may exhibit superior defensiveness to infection compared to synthetic spacers selected empirically. The methods and compositions provided herein thus provide an additional advantage of enabling the bacteria to naturally, e.g., by internal mechanisms, acquire spacers.

In some aspects, the compositions and methods provided herein allow for removal and/or control, e.g., transcriptional control, of the compositions for enabling adaptation in the organism. The ability to remove and/or control such compositions offers a unique element of control over the timing, e.g., initiation and/or duration, for enabling adaptation. Thus, a non-adapting CRISPR-Cas system may be enabled to adapt only for a period of time during which the bacteria containing the non-adapting CRISPR-Cas system is challenged with nucleic acids of interest, e.g., target nucleic acids. In some cases, the period of time is only for a duration sufficient to achieve a desirable phenotype. For example, in cases where a desirable phenotype is selected following stresses or selective pressures, the period of time during which adaptation is enabled is a duration sufficient to achieve an adapted non-adapting CRISPR-Cas system that can be used to produce a bacteria with the desirable phenotype. After challenge and/or emergence of a desirable phenotype, the compositions for enabling adaptation may be removed or otherwise controlled (e.g., transcriptionally controlled) to prevent further adaptation and/or undesirable spacer acquisition. The methods and compositions provided herein thus produce an adapted non-adapting CRISPR-Cas system having a known, e.g., customized, acquired immune profile, where the adapted non-adapting CRISPR-Cas system cannot acquire new spacers without performing the methods described herein. Thus, the methods and compositions provided herein produce an adapted non-adapting CRISPR-Cas system. In some embodiments, the adapted non-adapting CRISPR-Cas system may be introduced to other bacteria, thereby conferring resistance, e.g., phage resistance, MGE resistance, or desirable phenotypes, to the bacteria. The ability to introduce adapted non-adapting CRISPR-Cas systems to other bacteria, e.g., a recipient bacterial strain, provides an advantage in that strains of bacteria having resistance and/or desirable phenotypes can be produced without having to perform steps of subjecting the bacterial strain to invasive nucleic acids and/or stressors or selective pressures. The ability to produce an adapted non-adapting CRISPR-Cas system with customized immunity that can be introduced into bacteria may decrease the amount of time and effort needed to create strains with specific resistances and/or desirable phenotypes. The ability to quickly produce strains with specific resistances and/or desirable phenotypes may be particularly useful in the food industry, e.g., where phage infection is prevalent.

The compositions and methods provided herein allow for the controlled enablement of adaptation in non-adapting CRISPR-Cas systems without altering the features that render the CRISPR-Cas system incapable of adaptation. The composition and methods described herein can produce bacteria with naturally customized immune systems. In some cases, the immune systems may be customized to produce bacteria suitable, e.g., having desirable phenotypes, for a given process, e.g., food manufacturing.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a”, “an”, and “the” include plural referents unless the context clearly dictates otherwise. For example, “a” or “an” include “at least one” or “one or more.”

The terms “comprising”, “comprises” and “comprised of” as used herein are synonymous with “including”, “includes” or “containing”, “contains”, and grammatical variants thereof, are inclusive or open-ended and do not exclude additional, non-recited members, elements or method steps.

The terms “comprising”, “comprises” and “comprised of”, “including”, “includes” or “containing”, “contains”, and grammatical variants thereof also include the term “consisting of”.

All publications, including patent documents, scientific articles and databases, referred to in this application are incorporated by reference in their entirety for all purposes to the same extent as if each individual publication were individually incorporated by reference. Nothing herein is to be construed as an admission that such publications constitute prior art to the claims appended hereto. If a definition set forth herein is contrary to or otherwise inconsistent with a definition set forth in the patents, applications, published applications and other publications that are herein incorporated by reference, the definition set forth herein prevails over the definition that is incorporated herein by reference.

### I. DEFINITIONS

As used herein, “microorganism” or “microbe” refers to a bacterium, a fungus, a virus, a protozoan, and other microbes or microscopic organisms.

As used herein, “bacteria” refers to any of the prokaryotic microorganisms that exist as a single cell or in a cluster or aggregate of single cells.

As used here in the term “probiotic” refers to a composition for consumption by humans (e.g., as an or as a component of food) that contains viable (i.e. live) microorganisms, i.e., microorganisms that are capable of living and reproducing that, when administered in adequate amounts, confer a health benefit on a subject (see Hill et al. 2014 Nature Revs Gastro & Hep 11, 506-514, incorporated by reference herein in its entirety). A probiotic may contain one or more (such as any of 1, 2, 3, or 4) of any of the bacteria and strains thereof described herein. Probiotics may be distinguished from bacterial compositions that have been killed, for example, by pasteurization or heat treatment. Use of non-viable bacterial compositions is also contemplated in certain embodiments of the methods disclosed herein.

By “at least one strain,” is meant a single strain but also mixtures of strains comprising at least two strains of microorganisms, e.g., bacteria. By “a mixture of at least two strains,” is meant a mixture of two, three, four, five, six or even more strains. In some embodiments of a mixture of strains, the proportions can vary from 1% to 99%. When a mixture comprises more than two strains, the strains can be present in substantially equal proportions in the mixture or in different proportions.

For purposes of this disclosure, a “biologically pure strain” means a strain containing no other bacterial strains in quantities sufficient to interfere with replication of the strain or to be detectable when assessed using techniques recognized in the field. The terms “bacterial strain” and “recipient bacterial strain” encompass a bacterial cell and a recipient bacterial cell, respectively.

When used in connection with the organisms and cultures described herein, the term “isolated” includes not only a biologically pure strain, but also any culture of organisms which is grown or maintained other than as it is found in nature. In some instances, isolated may be used to refer to nucleic acid sequences and/or polypeptides.

CRISPR-Cas system as referred to herein includes a CRISPR array and one or more cas genes. In some embodiments, CRISPR-Cas system is encoded by CRISPR-cas locus, i.e., a DNA segment, located in the bacterial genome. In some embodiments, CRISPR-Cas system is encoded by a CRISPR-cas locus, i.e., a DNA segment, located on a plasmid present a bacterium.

As used herein, the term “CRISPR array” refers to the DNA segment which includes all of the CRISPR repeats and spacers, starting with the first nucleotide of the first CRISPR repeat and ending with the last nucleotide of the last (terminal) CRISPR repeat. Typically, each spacer sequence in a CRISPR array is located between two repeats and consequently, a locus includes one more repeat than spacer sequence. In some embodiments, the CRISPR array may also include a CRISPR leader sequence.

As used herein, “CRISPR spacer,” “spacer sequence,” or “spacer” refer to the non-repetitive sequences that are located between two repeats in a CRISPR array. As used herein, “protospacer” refers to the sequence within the target nucleic acid which corresponds to a given CRISPR spacer. Spacer acquisition in many CRISPR-Cas systems requires recognition of a short protospacer adjacent motif (PAM) in the target nucleic acid. These motifs are located in the direct vicinity of the protospacer (typically less than 10 nucleotides outside of the sequence) and appear to be specific to each CRISPR-Cas system. In some embodiments, a PAM, e.g., a spacer acquisition motif (SAM) or target interference motif (TIM), is not required for spacer acquisition, for example according the compositions and methods described herein. In some embodiments of the present invention, a “spacer” refers to the nucleic acid segment that is flanked by two repeats. CRISPR spacer sequences often have significant homology to naturally occurring phage or plasmid sequences. In some embodiments, the spacer has significant homology with the genome of the host organism. In some embodiments, the spacer has homology to a plasmid contained in the organism, alternatively referred to herein as a resident plasmid. Typically, spacers are located between two identical or nearly identical repeat sequences. Thus, spacers often are identified by sequence analysis of the DNA segments located between two CRISPR repeats.

As used herein, the terms “CRISPR repeat,” “repeat sequence,” or “repeat” have the conventional meaning as used in the art—i.e., multiple, short, direct repeating sequences, which show little or no sequence variation within a given CRISPR array. Many repeat sequences are partially palindromic, having the potential to form stable, conserved secondary structures.

As used herein the term “repeat-spacer” refers to spacer sequence associated with at least one repeat sequence.

CRISPR leader sequence is located between the first nucleotide of the first repeat in CRISPR array and the stop codon of the last cas gene.

As used herein, “CRISPR trailer” refers to the non-coding sequence located directly downstream of the 3′ end of the CRISPR array—i.e., right after the last nucleotide of the last CRISPR repeat. This last CRISPR repeat is also referred to as a “terminal repeat.”

As used herein, the term “cas gene” (for CRISPR-associated) has its conventional meaning as used in the art where it refers to a gene that is coupled to, associated with, close to, or in the vicinity of a CRISPR array. The expression “cas gene” includes, but is not limited to, cas, csn, csm and cmr genes, depending upon the type of CRISPR-Cas system. Thus, the person skilled in the art can easily identify based on conventional protein comparison bioinformatics tools (such as BLAST), whether a gene associated with a CRISPR locus encodes a Cas protein characteristic of any CRISPR-Cas system. The expression “Cas protein” encompasses Cas, Csn, Csm and Cmr proteins, depending upon the type of CRISPR-Cas system.

As used herein, the term “bacteriophage” or “phage” has its conventional meaning as understood in the art—i.e., a virus that selectively infects one or more bacterial species.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide,” “nucleic acid sequence,” “nucleotide sequence,” and “nucleic acid fragment” are used interchangeably to denote a polymer of RNA and/or DNA and/or RNA-DNA that is single- or double-stranded, optionally containing synthetic, non-natural, or altered nucleotide bases. Nucleotides (usually found in their 5′-monophosphate form) are referred to by their single letter designation, for example: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide. Nucleic acid notation is generally known in the art. For polynucleotides, conservative variants include those sequences that, because of the degeneracy of the genetic code, encode the amino acid sequence of a polypeptide disclosed herein. Naturally occurring allelic variants such as these can be identified with the use of well-known molecular biology techniques, as, for example, with polymerase chain reaction (PCR) and hybridization techniques as outlined below.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein, “derived from” encompasses “originated from,” “obtained from,” or “isolated from.”

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Values and ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges may implicitly include the term “about” unless the context clearly dictates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

### I. COMPOSITIONS

Provided herein, inter alia, are CRISPR-Cas systems incapable of performing adaptation; compositions, e.g., polynucleotides, constructs, vectors, proteins, useful for enabling adaptation in such CRISPR-Cas systems; and bacteria containing the non-adapting CRISPR-Cas systems and/or compositions described herein. Also provided are non-adapting CRISPR-Cas systems that have undergone adaptation according to the methods described herein and bacteria containing such adapted non-adapting CRISPR-Cas systems.

In some embodiments, CRISPR-Cas systems that are incapable of adaptation have a deficiency in one or more proteins, e.g., Cas proteins, that participate in and/or are required for adaptation. For example, the CRISPR-Cas system may not encode or encode non-functioning proteins, e.g., Cas proteins, that participate in and/or are required for adaptation. Such CRISPR-Cas systems are referred to herein as “non-adapting CRISPR-Cas systems.” In some embodiments, the compositions for enabling adaptation cure the deficiencies of the non-adapting CRISPR-Cas system by providing proteins, e.g., Cas proteins, that participate in and/or are required to perform adaptation.

In some embodiments, the compositions described herein, including non-adapting CRISPR-Cas systems and adapted non-adapting CRISPR-Cas systems described herein (see, e.g., Sections I-A and I-C, respectively), are isolated and/or purified. “Isolated” or “purified” as used herein refers to compositions described herein, or functional fragments thereof, that are substantially or essentially free from components that normally accompany or interact with the composition, such as a component found in its naturally occurring environment. Thus, an isolated or purified composition or functional fragment thereof is substantially free of other cellular material, or culture medium when produced by recombinant techniques, or substantially free of chemical precursors or other chemicals when chemically synthesized.

By way of example, an isolated polynucleotide may be free of sequences (optimally protein encoding sequences) that naturally flank the polynucleotide (i.e., 20 sequences located at the 5′ and 3′ ends of the polynucleotide) in the genomic DNA of the organism from which the polynucleotide is derived. In various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides and nucleic acid sequences thereof.

An isolated or purified polypeptide that is substantially free of cellular material may include preparations of polypeptides having less than about 30%, 20%, 10%, 5%, or 1% (by dry weight) of contaminating protein. When the polypeptides disclosed herein or functional fragments thereof are recombinantly produced, the culture medium may represent less than about 30%, 20%, 10%, 5%, or 1% (by dry weight) of chemical precursors or non-protein-of-interest chemicals.

Functional fragments of the compositions disclosed herein are also provided. For example, functional fragments include a portion of a polynucleotide (nucleic acid sequence) or a portion of an amino acid sequence (polypeptide) and hence protein encoded thereby. Functional fragments of a polynucleotide may encode protein fragments that retain the biological activity of the native protein, or fragments of a polynucleotide may retain the biological activity of the full size polynucleotide; these fragments are referred to herein as “functional fragments.” The terms “functional fragment,” “active fragment,” “fragment that is functionally equivalent,” and “functionally equivalent fragment” are used interchangeably herein.

Compositions disclosed herein include isolated and/or purified polynucleotides and polypeptides encoding proteins, e.g., Cas polypeptides, capable of enabling adaptation in CRISPR-Cas systems incapable of adapting. In addition, isolated and/or purified non-adapted CRISPR-Cas systems and adapted non-adapting CRISPR-Cas systems (e.g., adapted according to the methods described herein) are also provided.

Any form of composition, e.g., nucleic acid, construct, vector (e.g., plasmid), protein, etc., and method of delivery, e.g., transformation, conjugation, cell fusion, biolistic delivery, cell penetration, etc., known in the art and capable of introducing the compositions described herein to bacteria is contemplated for use herein.

### A. Non-Adapting CRISPR-Cas Systems

The adaptation function of a CRISPR-Cas system may be assayed by exposing to a phage a bacterial strain containing the CRISPR-Cas system (said bacterial strain being sensitive to said phage), selecting bacteriophage-resistant strains (i.e., strains which are resistant to this phage), and determining whether the resistance is conferred by the addition—in the CRISPR array of said CRISPR-Cas system—of at least one spacer sequence, e.g., a spacer sequence with nucleotide sequence identity to the phage. In some embodiments, a non-adapting CRISPR-Cas system is identified by determining that the resistance is not conferred by the addition of at least one spacer sequence with nucleotide sequence identity to the phage in the CRISPR array. In some embodiments, a non-adapting CRISPR-Cas system is identified by a lack of bacteriophage-resistant strains, e.g., after phage challenge.

A non-adapting CRISPR-Cas system may also be identified or, for example if assessed according to a functional method, e.g., as described above, further characterized by molecular methods known in the art. For example, the entire or portions of the CRISPR-Cas system may be sequenced to determine whether the CRISPR-Cas system codes for polypeptides that participate in and/or are required for adaptation. In some embodiments, sequences of all or portions of the CRISPR-Cas system may be compared to CRISPR-Cas systems known to perform adaptation. In some cases, non-adapting CRISPR-Cas systems may be identified by the lack of encoded proteins and/or the encoding of non-functioning proteins known to participate in and/or be required for adaptation.

In some cases, the non-adapting CRISPR-Cas system is incapable of adaptation because the system does not encode, does not express, or does not express a functional form of one or more Cas proteins that participate in and/or are required to perform adaptation. In some embodiments, the compositions provided herein cure the deficiencies of the non-adapting CRISPR-Cas system by providing the one or more Cas proteins that participate in and/or are required for CRISPR adaptation.

In some embodiments, the non-adapting CRISPR-Cas system does not encode one or more Cas proteins that participate in and/or are required for adaptation. For example, the non-adapting CRISPR-Cas system does not contain genes encoding one or more Cas proteins that participate in and/or are required for adaptation. In some embodiments, a Clustered Regularly Interspaced Short Palindromic Repeats (CRISPR)-associated protein 1 (Cas1) polypeptide participates in and/or is required for adaptation. In some embodiments, a CRISPR-associated endoribonuclease Cas2 (Cas2) polypeptide participates in and/or is required for adaptation. In some embodiments, a Cas1 or Cas2 polypeptide participates in and/or is required for adaptation. In some embodiments, Cas1 and Cas2 polypeptides participate in and/or are required for adaptation.

In some embodiments, the non-adapting CRISPR-Cas system does not encode a Cas1 polypeptide. In some embodiments, the non-adapting CRISPR-Cas system does not encode a Cas2 polypeptide. In some embodiments, the non-adapting CRISPR-Cas system does not encode a Cas1 or a Cas2 polypeptide. In some embodiments, the non-adapting CRISPR-Cas system does not encode Cas1 and Cas2 polypeptides.

In some cases, the non-adapting CRISPR-Cas system encodes one or more Cas proteins that are functionally incapable of performing adaptation. For example, the non-adapting CRISPR-Cas system does not contain genes encoding functional, e.g., biologically active forms, of one or more Cas proteins that participate in and/or are required for adaptation. In this case, the one or more Cas proteins are expressed but are functionally incapable of performing adaptation. A polypeptide that is incapable of performing a prescribed biological function may be referred to herein as a non-functioning polypeptide or protein. Non-functioning polypeptides may be identified by comparing their biological behavior to polypeptides known to participate and/or are required to perform a biological function, e.g., adaptation. Non-functioning polypeptides may have reduced activity, e.g., 50%, 60%, 70%. 80%, 90%, 95%, 96%, 97%, 98%, 99% reduced activity or no activity, compared to a functional control protein. Methods of assessing functional activity are known in the art. Non-limiting examples of assessing functional activity include PCR to detect spacer acquisition, e.g., in a culture population, or sequence of a culture population to identify newly acquired spacers.

In some embodiments, the non-adapting CRISPR-Cas system encodes a non-functioning Cas1 protein. In some embodiments, the non-adapting CRISPR-Cas system encodes a non-functioning Cas2 polypeptide. In some embodiments, the non-adapting CRISPR-Cas system encodes a non-functioning Cas1 polypeptide and a non-functioning Cas2 polypeptide. In some embodiments, the non-adapting CRISPR-Cas system encodes a non-functioning Cas1 protein and does not encode a Cas2 polypeptide. In some embodiments, the non-adapting CRISPR-Cas system encodes a non-functioning Cas2 protein and does not encode a Cas1 polypeptide. In some embodiments, the non-functioning protein (e.g., Cas1 and/or Cas2) is a truncated protein. In some embodiments, the non-functioning protein is a Cas1 and/or Cas2 protein that lacks the ability to facilitate spacer acquisition via adaptation.

In some embodiments, the non-adapting CRISPR-Cas system is a CRISPR-Cas system from a bacterium of the genus Lactococcus. In some embodiments, the non-adapting CRISPR-Cas system is a CRISPR-Cas system of the species L. cremoris. In some embodiments, the non-adapting CRISPR-Cas system is a CRISPR-Cas system from a subspecies or biovar of L. cremoris. In some embodiments, the non-adapting CRISPR-Cas system is a CRISPR-Cas system from an L. cremoris subsp cremoris. In some embodiments, the non-adapting CRISPR-Cas system is a CRISPR-Cas system of the species L. lactis. In some embodiments, the non-adapting CRISPR-Cas system is a CRISPR-Cas system from a subspecies or biovar of L. lactis.

In some embodiments, the non-adapting CRISPR-Cas system is a CRISPR-Cas system from an L. lactis subsp lactis. In some embodiments, the non-adapting CRISPR-Cas system is a type Ill-A CRISPR-Cas system. In some embodiments, the non-adapting CRISPR-Cas system is a type III-A CRISPR-Cas system from a species or strain of Lactococcus, e.g., L. cremoris subsp cremoris.

In some embodiments, the non-adapting CRISPR-Cas system includes the nucleic acid sequence set forth by SEQ ID NO:25. In some embodiments, the non-adapting CRISPR-Cas system includes a nucleic acid sequence having at least 70%, 75%, 80%, 85%, 90%, 91%, 92%, 93%, 94%, 95%, 96%, 97%, 98%, or 99% sequence identity to the sequence set forth in SEQ ID NO:25. In some embodiments, the non-adapting CRISPR-Cas system includes a nucleic acid sequence having at least 80%, 85%, 90%, 91%, 92%, 93%, 94%, 95%, 96%, 97%, 98%, or 99% sequence identity to the sequence set forth in SEQ ID NO:25. In some embodiments, the non-adapting CRISPR-Cas system includes a nucleic acid sequence having at least 90%, 91%, 92%, 93%, 94%, 95%, 96%, 97%, 98%, or 99% sequence identity to the sequence set forth in SEQ ID NO:25. In some embodiments, the non-adapting CRISPR-Cas system includes a nucleic acid sequence having at least 95%, 96%, 97%, 98%, or 99% sequence identity to the sequence set forth in SEQ ID NO:25. In some embodiments, the non-adapting CRISPR-Cas system encodes a Cas1 polypeptide having the sequence set forth by SEQ ID NO:24 In some embodiments, the non-adapting CRISPR-Cas system a Cas1 polypeptide having at least 70%, 75%, 80%, 85%, 90%, 91%, 92%, 93%, 94%, 95%, 96%, 97%, 98%, or 99% sequence identity to the sequence set forth in SEQ ID NO:24. In some embodiments, the non-adapting CRISPR-Cas system encodes a polypeptide having at least 80%, 85%, 90%, 91%, 92%, 93%, 94%, 95%, 96%, 97%, 98%, or 99% sequence identity to the sequence set forth in SEQ ID NO:24. In some embodiments, the non-adapting CRISPR-Cas system encodes a Cas1 polypeptide having at least 90%, 91%, 92%, 93%, 94%, 95%, 96%, 97%, 98%, or 99% sequence identity to the sequence set forth in SEQ ID NO:24. In some embodiments, the non-adapting CRISPR-Cas system encodes a Cas1 polypeptide having at least 95%, 96%, 97%, 98%, or 99% sequence identity to the sequence set forth in SEQ ID NO:24. In some embodiments, the non-adapting CRISPR-Cas system does not encode a Cas2 polypeptide.

In some embodiments, the non-adapting CRISPR-Cas system is contained in a plasmid. In some embodiments, the plasmid is a conjugative plasmid. In some embodiments, the plasmid is a non-conjugative plasmid. In some embodiments, the plasmid is a mobilizable plasmid. In some embodiments, the non-adapting CRISPR-Cas system is contained in the p537CR plasmid.

### B. Compositions for Enabling Adaptation

Provided herein are composition for enabling adaptation in non-adapting CRISPR-Cas systems, for example as described in Section I-A above. In some embodiments, the compositions for enabling adaptation cure, e.g., temporarily, the deficiencies of the non-adapting CRISPR-Cas system by providing proteins, e.g., Cas proteins, that participate in and/or are required to perform adaptation.

The compositions for enabling adaptation in non-adapting CRISPR-Cas systems may be provided in any form, e.g., nucleic acid, construct, vector (e.g., plasmid), protein, etc. In the event that the compositions are provided to a cell, e.g., bacteria (see, e.g., Section I-D), the compositions may be delivered by any means necessary, e.g., transformation, conjugation, cell fusion, biolistic delivery, cell penetration, etc., known in the art capable of introducing the compositions described herein to a cell.

### 1. Polynucleotides and Constructs

In some cases, the compositions for enabling adaptation in a non-adapting CRISPR-Cas system may be in the form of polynucleotides. For example, the polynucleotide may be or may contain a nucleic acid sequence encoding one or more proteins useful for adaptation. In some embodiments, the polynucleotide is or includes a nucleic acid sequence encoding a Cas polypeptide known to participate in and/or be necessary for CRISPR adaptation, e.g., a Cas1 and/or Cas2 polypeptide. In some embodiments, the polynucleotides provided herein are recombinant polynucleotides.

In some embodiments, the polynucleotide is or includes a nucleic acid sequence encoding a Cas1 polypeptide. In some embodiments, the Cas1 polypeptide is a Cas1 polypeptide of a Lactococcus bacterial strain. In some embodiments, the Cas1 polypeptide is a Cas1 polypeptide of a Lactococcus bacterial strain that is able to perform adaptation. In some embodiments, the Cas1 polypeptide is a Cas1 polypeptide of a Lactococcus raffinolactis bacterial strain. In some embodiments, the Lactococcus raffinolactis bacterial strain is the strain deposited under accession number CP047616. In some embodiments, the Cas1 polypeptide is a Cas1 polypeptide of a Lactococcus lactis bacterial strain. In some embodiments, the Cas1 polypeptide is a Cas1 polypeptide of a Lactococcus cremoris bacterial strain. In some embodiments, the L. lactis and L. cremoris bacterial strains are strains that are able to perform adaptation. In some embodiments, the L. lactis bacterial strain able to perform adaptation is an L. lactis subsp lactis bacterial strain. In some embodiments, the L. cremoris bacterial strain able to perform adaptation is an L. cremoris subsp cremoris bacterial strain.

In some embodiments, the polynucleotide is or includes a nucleic acid sequence encoding a Cas2 polypeptide. In some embodiments, the Cas2 polypeptide is a Cas2 polypeptide of a Lactococcus bacterial strain. In some embodiments, the Cas2 polypeptide is a Cas2 polypeptide of a Lactococcus bacterial strain that is able to perform adaptation. In some embodiments, the Cas2 polypeptide is a Cas2 polypeptide of a Lactococcus raffinolactis bacterial strain. In some embodiments, the Lactococcus raffinolactis bacterial strain is the strain deposited under accession number CP047616. In some embodiments, the Cas2 polypeptide is a Cas2 polypeptide of a Lactococcus lactis bacterial strain. In some embodiments, the Cas2 polypeptide is a Cas2 polypeptide of a Lactococcus cremoris bacterial strain. In some embodiments, the L. lactis and L. cremoris bacterial strains are strains that are able to perform adaptation. In some embodiments, the L. lactis bacterial strain able to perform adaptation is an L. lactis subsp lactis bacterial strain. In some embodiments, the L. cremoris bacterial strain able to perform adaptation is an L. cremoris subsp cremoris bacterial strain.

In some embodiments, the Cas1 and Cas2 polypeptides encoded by the nucleic acid sequences are from a single genus of bacteria. In some embodiments, the Cas1 and Cas2 polypeptides encoded by the nucleic acid sequences are polypeptides from the same species of bacteria. In some embodiments, the Cas1 and Cas2 polypeptides encoded by the nucleic acid sequences are polypeptides from the same strain of bacteria. In some embodiments, the Cas1 and Cas2 polypeptides encoded by the nucleic acid sequences are polypeptides from different species of bacteria. In some embodiments, the Cas1 and Cas2 polypeptides encoded by the nucleic acid sequences are polypeptides from different strains of bacteria. In some embodiments, the genus of bacteria is Lactococcus. In some embodiments, the species of bacteria or strain of bacteria is selected from Lactococcus lactis, Lactococcus cremoris, and/or Lactococcus raffinolactis. In some embodiments, the species of bacteria or strain of bacteria is a Lactococcus lactis. In some embodiments, the species of bacteria or strain of bacteria is a Lactococcus cremoris. In some embodiments, the L. lactis and L. cremoris bacterial strains are strains that are able to perform adaptation. In some embodiments, the L. lactis bacterial strain able to perform adaptation is an L. lactis subsp lactis bacterial strain. In some embodiments, the L. cremoris bacterial strain able to perform adaptation is an L. cremoris subsp cremoris bacterial strain. In some embodiments, the species of bacteria or strain of bacteria is a Lactococcus raffinolactis. In some embodiments, the Lactococcus raffinolactis bacterial strain is the strain deposited under accession number CP047616.

In some embodiments, the Cas1 polypeptide is encoded by a nucleic acid sequence having or including the sequence set forth in SEQ ID NO:3. In some embodiments, the Cas1 is encoded by a nucleic acid sequence having or including at least 70%, 75%, 80%, 85%, 90%, 91%, 92%, 93%, 94%, 95%, 96%, 97%, 98%, or 99% sequence identity to the sequence set forth in SEQ ID NO:3. In some embodiments, the Cas1 is encoded by a nucleic acid sequence having or including at least 80%, 85%, 90%, 91%, 92%, 93%, 94%, 95%, 96%, 97%, 98%, or 99% sequence identity to the sequence set forth in SEQ ID NO:3. In some embodiments, the Cas1 is encoded by a nucleic acid sequence having or including at least 90%, 91%, 92%, 93%, 94%, 95%, 96%, 97%, 98%, or 99% sequence identity to the sequence set forth in SEQ ID NO:3. In some embodiments, the Cas1 is encoded by a nucleic acid sequence having or including at least 95%, 96%, 97%, 98%, or 99% sequence identity to the sequence set forth in SEQ ID NO:3. In some embodiments, the Cas1 polypeptide encoded by the nucleic acid sequence has or includes an amino acid sequence set forth in SEQ ID NO:2. In some embodiments, the Cas1 polypeptide encoded by the nucleic acid sequence has or includes an amino acid sequence having at least 70%, 75%, 80%, 85%, 90%, 91%, 92%, 93%, 94%, 95%, 96%, 97%, 98%, or 99% sequence identity to the sequence set forth in SEQ ID NO:2. In some embodiments, the Cas1 polypeptide encoded by the nucleic acid sequence has or includes an amino acid sequence having at least 80%, 85%, 90%, 91%, 92%, 93%, 94%, 95%, 96%, 97%, 98%, or 99% sequence identity to the sequence set forth in SEQ ID NO:2. In some embodiments, the Cas1 polypeptide encoded by the nucleic acid sequence has or includes an amino acid sequence having at least 90%, 91%, 92%, 93%, 94%, 95%, 96%, 97%, 98%, or 99% sequence identity to the sequence set forth in SEQ ID NO:2. In some embodiments, the Cas1 polypeptide encoded by the nucleic acid sequence has or includes an amino acid sequence having at least 95%, 96%, 97%, 98%, or 99% sequence identity to the sequence set forth in SEQ ID NO:2. In some embodiments, the polynucleotide encodes any of the Cas1 polypeptides described herein.

In some embodiments, the Cas2 polypeptide is encoded by a nucleic acid sequence having or including the sequence set forth in SEQ ID NO:5. In some embodiments, the Cas2 is encoded by a nucleic acid sequence having or including at least 70%, 75%, 80%, 85%, 90%, 91%, 92%, 93%, 94%, 95%, 96%, 97%, 98%, or 99% sequence identity to the sequence set forth in SEQ ID NO:5. In some embodiments, the Cas2 is encoded by a nucleic acid sequence having or including at least 80%, 85%, 90%, 91%, 92%, 93%, 94%, 95%, 96%, 97%, 98%, or 99% sequence identity to the sequence set forth in SEQ ID NO:5. In some embodiments, the Cas2 is encoded by a nucleic acid sequence having or including at least 90%, 91%, 92%, 93%, 94%, 95%, 96%, 97%, 98%, or 99% sequence identity to the sequence set forth in SEQ ID NO:5. In some embodiments, the Cas2 is encoded by a nucleic acid sequence having or including at least 95%, 96%, 97%, 98%, or 99% sequence identity to the sequence set forth in SEQ ID NO:5. In some embodiments, the Cas2 polypeptide encoded by the nucleic acid sequence has or includes an amino acid sequence set forth in SEQ ID NO:4. In some embodiments, the Cas2 polypeptide encoded by the nucleic acid sequence has or includes an amino acid sequence having at least 70%, 75%, 80%, 85%, 90%, 91%, 92%, 93%, 94%, 95%, 96%, 97%, 98%, or 99% sequence identity to the sequence set forth in SEQ ID NO:4. In some embodiments, the Cas2 polypeptide encoded by the nucleic acid sequence has or includes an amino acid sequence having at least 85%, 90%, 91%, 92%, 93%, 94%, 95%, 96%, 97%, 98%, or 99% sequence identity to the sequence set forth in SEQ ID NO:4. In some embodiments, the Cas2 polypeptide encoded by the nucleic acid sequence has or includes an amino acid sequence having at least 90%, 91%, 92%, 93%, 94%, 95%, 96%, 97%, 98%, or 99% sequence identity to the sequence set forth in SEQ ID NO:4. In some embodiments, the Cas2 polypeptide encoded by the nucleic acid sequence has or includes an amino acid sequence having at least 95%, 96%, 97%, 98%, or 99% sequence identity to the sequence set forth in SEQ ID NO:4. In some embodiments, the polynucleotide encodes any of the Cas2 polypeptides described herein.

In some embodiments, the nucleic acid sequence encoding Cas1 and the nucleic acid sequence encoding Cas2 are contained in separate polynucleotides. In some embodiments, the nucleic acid sequence encoding Cas1 and the nucleic acid sequence encoding Cas2 are contained in a single polynucleotide.

In some embodiments, the polynucleotide is or includes nucleic acid sequences encoding Cas1 and Cas2 polypeptides as described herein. In some embodiments, the Cas1 and Cas2 polypeptides encoded by the polynucleotide are from an L. lactis bacterial strain. In some embodiments, the Cas1 and Cas2 polypeptides encoded by the polynucleotide are from an L. lactis subsp lactis bacterial strain. In some embodiments, the Cas1 and Cas2 polypeptides are encoded by the polynucleotide are from an L. cremoris bacterial strain. In some embodiments, the Cas1 and Cas2 polypeptides are encoded by the polynucleotide are from an L. cremoris subsp cremoris bacterial strain. In some embodiments, the Cas1 and Cas2 polypeptides encoded by the polynucleotide are from an L. raffinolactis bacterial strain. In some embodiments, the Lactococcus raffinolactis bacterial strain is the strain deposited under accession number CP047616.

In some embodiments, the polynucleotide includes a nucleic acid sequence encoding a Cas1 polypeptide, the nucleic acid sequence having the sequence set forth by SEQ ID NO:3 or a sequence having at least 70%, 75%, 80%, 85%, 90%, 91%, 92%, 93%, 94%, 95%, 96%, 97%, 98%, or 99% sequence identity to the sequence set forth in SEQ ID NO:3, and a nucleic acid sequence encoding a Cas2 polypeptide, the nucleic acid sequence having the sequence set forth by SEQ ID NO:5 or a sequence having at least 70%, 75%, 80%, 85%, 90%, 91%, 92%, 93%, 94%, 95%, 96%, 97%, 98%, or 99% sequence identity to the sequence set forth in SEQ ID NO:5. In some embodiments, the polynucleotide includes a nucleic acid sequence encoding a Cas1 polypeptide, the nucleic acid sequence having the sequence set forth by SEQ ID NO:3 or a sequence having at least 80%, 85%, 90%, 91%, 92%, 93%, 94%, 95%, 96%, 97%, 98%, or 99% sequence identity to the sequence set forth in SEQ ID NO:3, and a nucleic acid sequence encoding a Cas2 polypeptide, the nucleic acid sequence having the sequence set forth by SEQ ID NO:5 or a sequence having at least 80%, 85%, 90%, 91%, 92%, 93%, 94%, 95%, 96%, 97%, 98%, or 99% sequence identity to the sequence set forth in SEQ ID NO:5. In some embodiments, the polynucleotide includes a nucleic acid sequence encoding a Cas1 polypeptide, the nucleic acid sequence having the sequence set forth by SEQ ID NO:3 or a sequence having at least 70%, 90%, 91%, 92%, 93%, 94%, 95%, 96%, 97%, 98%, or 99% sequence identity to the sequence set forth in SEQ ID NO:3, and a nucleic acid sequence encoding a Cas2 polypeptide, the nucleic acid sequence having the sequence set forth by SEQ ID NO:5 or a sequence having at least 90%, 91%, 92%, 93%, 94%, 95%, 96%, 97%, 98%, or 99% sequence identity to the sequence set forth in SEQ ID NO:5. In some embodiments, the polynucleotide includes a nucleic acid sequence encoding a Cas1 polypeptide, the nucleic acid sequence having the sequence set forth by SEQ ID NO:3 or a sequence having at least 95%, 96%, 97%, 98%, or 99% sequence identity to the sequence set forth in SEQ ID NO:3, and a nucleic acid sequence encoding a Cas2 polypeptide, the nucleic acid sequence having the sequence set forth by SEQ ID NO:5 or a sequence having at least 95%, 96%, 97%, 98%, or 99% sequence identity to the sequence set forth in SEQ ID NO:5.

In some embodiments, the polynucleotide encodes a Cas1 polypeptide having the sequence set forth by SEQ ID NO:2 or a sequence having at least 70%, 75%, 80%, 85%, 90%, 91%, 92%, 93%, 94%, 95%, 96%, 97%, 98%, or 99% sequence identity to the sequence set forth in SEQ ID NO:2, and a Cas2 polypeptide having the sequence set forth by SEQ ID NO:4 or a sequence having at least 70%, 75%, 80%, 85%, 90%, 91%, 92%, 93%, 94%, 95%, 96%, 97%, 98%, or 99% sequence identity to the sequence set forth in SEQ ID NO:4. In some embodiments, the polynucleotide encodes a Cas1 polypeptide having the sequence set forth by SEQ ID NO:2 or a sequence having at least 80%, 85%, 90%, 91%, 92%, 93%, 94%, 95%, 96%, 97%, 98%, or 99% sequence identity to the sequence set forth in SEQ ID NO:2, and a Cas2 polypeptide having the sequence set forth by SEQ ID NO:4 or a sequence having at least 80%, 85%, 90%, 91%, 92%, 93%, 94%, 95%, 96%, 97%, 98%, or 99% sequence identity to the sequence set forth in SEQ ID NO:4. In some embodiments, the polynucleotide encodes a Cas1 polypeptide having the sequence set forth by SEQ ID NO:2 or a sequence having at least 90%, 91%, 92%, 93%, 94%, 95%, 96%, 97%, 98%, or 99% sequence identity to the sequence set forth in SEQ ID NO:2, and a Cas2 polypeptide having the sequence set forth by SEQ ID NO:4 or a sequence having at least 90%, 91%, 92%, 93%, 94%, 95%, 96%, 97%, 98%, or 99% sequence identity to the sequence set forth in SEQ ID NO:4. In some embodiments, the polynucleotide encodes a Cas1 polypeptide encoded by the polynucleotide has or is the sequence set forth by SEQ ID NO:2 or a sequence having at least 95%, 96%, 97%, 98%, or 99% sequence identity to the sequence set forth in SEQ ID NO:2, and a Cas2 polypeptide having the sequence set forth by SEQ ID NO:4 or a sequence having at least 95%, 96%, 97%, 98%, or 99% sequence identity to the sequence set forth in SEQ ID NO:4. In some embodiments, the polynucleotide encodes a Cas1 and Cas2 polypeptide as disclosed herein. In some embodiments, the polynucleotide includes nucleic acid sequences disclosed herein that encode Cas1 and Cas2 polypeptides provided herein.

In some embodiments, when the Cas1 and Cas2 polypeptides are encoded by nucleic acid sequences contained in a single polynucleotide, the nucleic acid sequence encoding the Cas1 polypeptide is positioned 5′ to the nucleic acid sequence encoding the Cas2 polypeptide. In some embodiments, when the Cas1 and Cas2 polypeptides are encoded by nucleic acid sequences contained in a single polynucleotide, the nucleic acid sequence encoding the Cas2 polypeptide is positioned 5′ to the nucleic acid sequence encoding the Cas1 polypeptide. Any orientation of the nucleic acid sequences encoding the Cas1 and Cas2 polypeptides is contemplated herein.

In some embodiments, the polynucleotide is or includes a nucleic acid sequence having the sequence set forth by SEQ ID NO:1. In some embodiments, the polynucleotide is or includes a nucleic acid sequence having at least 70%, 75%, 80%, 85%, 90%, 91%, 92%, 93%, 94%, 95%, 96%, 97%, 98%, or 99% sequence identity to the sequence set forth in SEQ ID NO:1. In some embodiments, the polynucleotide is or includes a nucleic acid sequence having at least 80%, 85%, 90%, 91%, 92%, 93%, 94%, 95%, 96%, 97%, 98%, or 99% sequence identity to the sequence set forth in SEQ ID NO:1. In some embodiments, the polynucleotide is or includes a nucleic acid sequence having at least 90%, 91%, 92%, 93%, 94%, 95%, 96%, 97%, 98%, or 99% sequence identity to the sequence set forth in SEQ ID NO:1. In some embodiments, the polynucleotide is or includes a nucleic acid sequence having at least 95%, 96%, 97%, 98%, or 99% sequence identity to the sequence set forth in SEQ ID NO:1.

In some embodiments, the polynucleotides described herein are constructs. Construct and cassette may be used interchangeably herein to refer to polynucleotide sequences that are directly or indirectly linked, e.g., attached, to a regulatory sequence, such as a heterologous regulatory sequence, and/or a polycistronic element. An example of an indirect link is the provision of a suitable spacer group such as an intron sequence, such as the ShI-intron or the ADH intron, intermediate a promoter and a nucleic acid sequence described herein. In some cases, the terms do not cover the natural combination of the polynucleotide sequence coding for the protein ordinarily associated with the wild type gene promoter and when they are both in their natural environment.

The construct may further contain or express another gene, such as a marker allowing for the selection of the construct. Various markers exist which may be used, for example those markers that provide for antibiotic resistance—e.g. resistance to bacterial antibiotics—such as Chloramphenicol, Erythromycin, Ampicillin, Streptomycin and Tetracycline.

In some embodiments, the polynucleotide including a nucleic acid sequence encoding a Cas polypeptide as described herein (e.g., Cas1 polypeptide, Cas2 polypeptide), includes a heterologous regulatory sequence. In some embodiments, the polynucleotide is or includes a nucleic acid sequence encoding a Cas1 polypeptide as described herein operably linked to a heterologous regulatory sequence. In some embodiments, the polynucleotide is or includes a nucleic acid sequence encoding a Cas2 polypeptide as described herein operably linked to a heterologous regulatory sequence. In some embodiments, the heterologous regulatory sequence is a promoter, enhancer, or activator.

In some embodiments, the heterologous regulatory sequence is a promoter. A suitable promoter may be selected depending on the bacteria into which the polynucleotide will be introduced, e.g., a promoter recognizable by the RNA polymerase present in the particular bacteria used. In some embodiments, the promoter is a constitutive promoter. A constitutive promoter may be used to allow continuous transcription of the sequence to which it is operably linked.

In some embodiments, the promoter is an inducible promoter. The inducible promoter may be used to selectively control the expression of the encoded protein to which it is operably linked. Inducible promoters (also referred to as regulated promoters) include, for example, promoters induced or regulated by light, heat, stresses, sugars, peptides, and metal ions. A variety of inducible promoters are known in the art and useful in driving expression of the proteins provided herein. Such promoters include those induced by growth in particular sugars, such as L-arabinose, L-rhamnose, xylose, lactose and sucrose; promoters induced by antibiotics, such as tetracyclines or bacteriocin, e.g., nisin; promoters induced by other chemical compounds such as substituted benzenes, cyclohexanone-related compounds, e-caprolactam, propionate, thiostrepton, alkanes, and peptides; promoters induced by bacteriophages, e.g., a phage-inducible promoter, such as Φ31p, which has been described in Djordjevic and Klaenhammer 1997, Djordjevic et al., 1997, and Walker and Klaenhammer 2000; promoters induced by light, such as blue, red, or green light. For a review of inducible promoters see, e.g., Brautaset, el al., Microb Biotechnol. (2009) 2: 15-30.

In some embodiments, the promoter is a chemical-inducible promoter, where the application of a chemical induces expression. In some embodiments, the promoter is a phage-inducible promoter, where the presence of a phage induces expression. In some embodiments, the promoter is a light-inducible promoter, where application of specific wavelengths of light induces expression.

Non-limiting examples of promoters contemplated for use according to the compositions and methods described herein include L-arabinose inducible (araBAD, PBAD) promoter, any lac promoter, L-rhamnose inducible (rhaPBAD) promoter, T7 RNA polymerase promoter, trc promoter, tac promoter, Φ31p, lambda phage promoter (p_{L}p_{L}-9G-50), anhydrotetracycline-inducible (tetA) promoter, trp, lpp, phoA, recA, pro U, cst-1, cadA, nar, lpp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, GA, GB), Ptms, P43, rplK-rplA, ferredoxin promoter, and/or xylose promoter.

In some embodiments, the polynucleotide is or includes from 5′ to 3′: a heterologous regulatory sequence, such as a promoter described herein, and a nucleic acid sequence encoding a Cas1 polypeptide as described herein. In some embodiments, the polynucleotide is or includes from 5′ to 3′: a heterologous regulatory sequence, such as a promoter described herein, and a nucleic acid sequence encoding a Cas2 polypeptide described herein.

In some embodiments, for example when nucleic acid sequences encoding Cas1 and Cas2 polypeptides are contained in a single polynucleotide, the polynucleotide may include one or more heterologous regulatory sequences and/or polycistronic elements. A heterologous regulatory sequence and/or polycistronic elements may be useful to ensure that the nucleic acid sequences encoding the polypeptides (e.g., Cas1 polypeptide, Cas2 polypeptide) are expressed. In some embodiments, the heterologous regulatory sequence is a promoter, for example, as described herein. In some embodiments, the polycistronic element is a ribosome binding sequence, e.g., a Shine-Dalgarno sequence. In some embodiments, the polycistronic element is an internal ribosome entry site (IRES). In some embodiments, the polycistronic element is a ribosomal skip sequence or self-cleaving peptide, e.g., T2A, a P2A, an E2A, or an F2A element. In some embodiments, the polynucleotide contains the polycistronic element positioned between the nucleic acid sequences of the polynucleotide encoding the Cas1 and Cas2 polypeptides.

In some embodiments, the polynucleotide is or includes from 5′ to 3′: a heterologous regulatory sequence, such as a promoter described herein; a nucleic acid sequence encoding a Cas1 polypeptide as described herein; a polycistronic sequence as described herein; and a nucleic acid sequence encoding a Cas2 polypeptide as described herein. In some embodiments, the polynucleotide is or includes from 5′ to 3′: a heterologous regulatory sequence, such as a promoter described herein; a nucleic acid sequence encoding a Cas2 polypeptide as described herein; a polycistronic sequence as described herein; and a nucleic acid sequence encoding a Cas1 polypeptide as described herein. In some embodiments, the polynucleotide is or includes from 5′ to 3′: a first heterologous regulatory sequence, such as a promoter described herein; a nucleic acid sequence encoding a Cas1 polypeptide as described herein; a second heterologous regulatory sequence, such as a promoter as described herein; and a nucleic acid sequence encoding a Cas2 polypeptide as described herein. In some embodiments, the polynucleotide is or includes from 5′ to 3′: a first heterologous regulatory sequence, such as a promoter described herein; a nucleic acid sequence encoding a Cas2 polypeptide as described herein; a second heterologous regulatory sequence, such as a promoter as described herein; and a nucleic acid sequence encoding a Cas1 polypeptide as described herein.

In some embodiments, the polynucleotides provided herein are recombinant nucleic acid sequences. In some embodiments, the polynucleotides provided herein are labile. Labile polynucleotides may include labile nucleosides, for example as described in published application US 2002/0127575, which is incorporated herein by reference in its entirety.

### 2. Vectors

The polynucleotides and constructs described herein (see, e.g., Section I-B1) may be present in a vector. As used herein, vector refers to any nucleic acid molecule into which another nucleic acid molecule (e.g., nucleic acid sequence encoding a Cas polypeptide) can be inserted and which can be introduced into and optionally replicate within a bacterial strain. In some embodiments, the vector may be referred to as an expression vector, meaning that the coding nucleic acid sequences contained in the vector are capable of in vivo or in vitro expression. The choice of vector, e.g. plasmid, cosmid, virus or phage vector, will often depend on the host cell, e.g., bacteria, into which it is to be introduced. In some embodiment, the vector is a plasmid.

The vectors may contain one or more selectable marker genes—such as a gene which confers antibiotic resistance e.g. ampicillin, kanamycin, chloramphenicol or tetracyclin resistance. Alternatively, the selection may be accomplished by co-transformation (as described in WO91/17243).

The vector may further comprise a nucleotide sequence enabling the vector to replicate in the host cell in question. Examples of such sequences are the origins of replication of plasmids pWV01, pUC19, pACYC177, pUBI 10, pE194, pAMBI and pIJ702.

In some embodiments, provided herein are vectors containing the polynucleotides or constructs described herein, see, e.g., Section I-B1. In some embodiments, the vector contains a polynucleotide which encodes a Cas protein described herein, see, e.g., Section I-B1. In some embodiments, the vector contains a heterologous regulatory sequence. In some embodiments, the heterologous regulatory sequence is a promoter as described herein, see, e.g., Section I-B1.

The vectors provided herein may be introduced into a bacterial strain as described herein (see, e.g., Section II-A). In some embodiments, the vector can be further cured or otherwise removed from the bacterial strain following introduction. In some embodiments, the vector is cured naturally through cell division. In some embodiments, the vector is cured by treating the bacteria with chemical or physical agents. Exemplary means of curing plasmids from bacteria include, but are not limited to, treatment with acridine mutagens, ion and ionizing radiation, thyme starvation, antibiotics and growth above optimum temperature, pH or extreme environmental conditions. In some embodiments, the vectors are labile. For example, in some cases, propagation of the vector may be heat-sensitive or require the presence of an antitoxin. In some embodiments, the vector may encode a conditionally lethal gene. Vectors for such use are known in the art and may be selected accordingly. Non-limiting example of vectors contemplated herein include pGhost9, pTRK989, pNZ124 (Boca Scientific Inc, Westwood, MA), and pNice (Boca Scientific Inc, Westwood, MA).

### 3. Polypeptides

In some embodiments, the compositions provided herein for enabling adaptation are amino acid sequences. In some embodiments, the amino acid sequence is or includes a Cas1 polypeptide as described herein (see, e.g., Section I-B1). In some embodiments, the amino acid sequence is or includes a Cas2 polypeptide as described herein (see, e.g., Section I-1). In some embodiments, the amino acid sequence is or includes a Cas1 and a Cas2 polypeptide as described herein (see, e.g., Section I-1). In some embodiments, the Cas1 and Cas2 polypeptides are contained in a single amino acid sequence. In this case, the use of spacers and/or linkers may be used to ensure that the proteins fold and/or interact to exhibit functional behavior (e.g., adaptation). In some embodiments, the Cas1 and Cas2 polypeptides are contained in separate amino acid sequences.

The polypeptides disclosed herein may be altered in various ways including amino acid substitutions, deletions, truncations, and insertions. Methods for such manipulations are generally known in the art. For example, amino acid sequence variants and fragments of polypeptides can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing 15 Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.). Conservative substitutions, such as exchanging one amino acid with another having similar properties, may be optimal.

### C. Adapted Non-Adapting CRISPR-Cas Systems

Also provided herein are non-adapting CRISPR-Cas systems that have undergone adaptation according the methods provided herein (see, Section II). Non-adapting CRISPR-Cas systems, such as those described in Section I-A, having undergone successful adaptation are referred to herein as “adapted non-adapting CRISPR-Cas systems.” In some embodiments, the adapted non-adapting CRISPR-Cas system is a non-adapting CRISPR-Cas system according to any of the embodiments of Section I-A, that contains a spacer sequence, e.g., in the CRISPR array, that is not present in the non-adapting CRISPR-Cas system prior to undergoing the methods described herein to enable adaptation. Methods of detecting new spacers in a non-adapting CRISPR-Cas system may include PCR, DNA-DNA hybridization (or DNA-RNA hybridization e.g., using DNA or RNA probes that could be synthetic, labelled oligonucleotides, for example). DNA microarrays may also be used. In some embodiments, the sequence of the adapted non-adapting CRISPR-Cas system is compared to the sequence of the original non-adapting CRISPR-Cas system, e.g., a non-adapting CRISPR-Cas system prior to performing the methods of enabling adaptation described herein, to determine the presence of a new spacer. Exemplary methods of detecting new spacers include, but are not limited to, those described in Example 1 below and DNA sequencing.

In some embodiments, the adapted non-adapting CRISPR-Cas system is a non-adapting CRISPR-Cas system as described in Section I-A, that includes at least one new spacer sequence. In some embodiments, the adapted CRISPR-Cas system contains 1, 2, 3, 4, 5, 6, 7, 8, 9, 10, or more new spacers sequences. In some embodiments, the adapted CRISPR-Cas system contains about 1 to about 10 new spacer sequences. In some embodiments, the adapted CRISPR-Cas system contains about 1 to about 5 new spacer sequences. In some embodiments, the adapted CRISPR-Cas system contains about 1 to about 4 new spacer sequences. In some embodiments, the adapted CRISPR-Cas system contains about 1 to about 3 new spacer sequences. In some embodiments, the adapted CRISPR-Cas system contains about 1 to about 2 new spacer sequences. In some embodiments, the adapted CRISPR-Cas system contains 1 new spacer sequence. In some embodiments, the adapted CRISPR-Cas system contains 2 new spacer sequence. In some embodiments, the adapted CRISPR-Cas system contains 3 new spacer sequence. In some embodiments, the adapted CRISPR-Cas system contains 4 new spacer sequence. In some embodiments, the adapted CRISPR-Cas system contains 5 new spacer sequence. According to the methods provided herein, in some cases, it is possible to add as many new spacers as needed to achieve a desired immune profile.

In some embodiments, the new spacer sequence can be used in the processes of maturation and interference. Spacer sequences that can be used in such processes as maturation and interference may be referred to herein as spacer sequences active against a target nucleic acid. In some embodiments, the new spacer sequence is active against a target nucleic acid. embodiment, the target nucleic acid is a foreign nucleic acid. In some embodiments, the target nucleic acid is a DNA. In some embodiments, the DNA is double-stranded DNA. In some embodiments, the DNA is single-stranded DNA. In some embodiments, the target nucleic acid is an RNA. In some embodiments, said target nucleic acid is a chromosomal DNA sequence (i.e., a sequence present in the chromosome of the bacterial cell). In some embodiments, said target nucleic acid is a DNA sequence present in a plasmid, e.g., resident plasmid, of the bacterial cell. In some embodiments, the target nucleic acid is a transcript (e.g., a transcript expressed by the bacterial cell). In some embodiments, the new spacer is active against the target nucleic acid sequence per se. In some embodiments, the new spacer is active against a transcription product of the target nucleic acid sequence—such as a transcript of the target nucleic acid sequence [e.g. an RNA (e.g. mRNA)]. Examples of target nucleic acid include, but are not limited to, a bacteriophage genome, the transcription product of a bacteriophage genome, a plasmid, a resident plasmid, a chromosomal sequence, a mobile genetic element, a transposable element or an insertion sequence. In some embodiment, the target nucleic acid is selected from a bacteriophage genome, the transcription product of a bacteriophage genome, a plasmid, a resident plasmid, a chromosomal sequence, a mobile genetic element, a transposable element and an insertion sequence. As used herein, a self-targeting spacer refers to a spacer sequence corresponding to a protospacer (as defined herein) the sequence of which is present in the genome of said bacteria (associated with a PAM if required) or on a plasmid contained in the bacteria, e.g., resident plasmid. In some embodiments, the target nucleic acid is a bacteriophage genome or the transcription product of a bacteriophage genome. In some embodiment, the target nucleic acid is a plasmid. In some embodiment, the target nucleic acid is a chromosomal sequence.

In some embodiments, the target nucleic acid is a nucleic acid from a bacteriophage. In some embodiments, the target nucleic acid is or is derivable from a bacteriophage. Many bacteriophages are specific to a particular genus or species or strain of cell. The bacteriophage may be a lytic bacteriophage or a lysogenic bacteriophage. A lytic bacteriophage is one that follows the lytic pathway through completion of the lytic cycle, rather than entering the lysogenic pathway. A lytic bacteriophage undergoes viral replication leading to lysis of the cell membrane, destruction of the cell, and release of progeny bacteriophage particles capable of infecting other cells. By way of example, the bacteriophage include, but are not limited to, those bacteriophage capable of infecting bacteria belonging to the following genera: Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, Brucella and Xanthomonas. By way of further example, the bacteriophage include, but are not limited to, those bacteriophage capable of infecting (or transducing) lactic acid bacteria species, a Bifidobacterium species, a Brevibacterium species, a Propionibacterium species, a Lactococcus species, a Streptococcus species, a Lactobacillus species including the Lactobacillus acidophilus, Enterococcus species, Pediococcus species, a Leuconostoc species and Oenococcus species.

By way of further example, the bacteriophage include, but are not limited to, those bacteriophage capable of infecting Lactococcus lactis, Lactococcus lactis subsp lactis, Lactococcus cremoris, Lactococcus cremoris subsp cremoris, Lactococcus lactis subsp. lactis biovar diacetylactis, Streptococcus thermophilus, Lactobacillus delbrueckii subsp. bulgaricus, Lactobacillus helveticus, Bifidobacterium lactis, Lactobacillus acidophilus, Lacticaseibacillus casei, Bifidobacterium infantis, Lacticaseibacillus paracasei, Lactobacillus salivarins, Lactiplantibacillus plantarum, Lactobacillus reuteri, Lactobacillus gasseri, Lactobacillus johnsonii or Bifidobacterium longum.

By way of further example, the bacteriophages include, but are not limited to, those bacteriophage capable of infecting any fermentative bacteria susceptible to disruption by bacteriophage infection, including but not limited to processes for the production of antibiotics, amino acids, and solvents. Products produced by fermentation which are known to have encountered bacteriophage infection, and the corresponding infected fermentation bacteria, include, but are not limited to, Cheddar and cottage cheese (Lactococcus lactis subsp lactis, Lactococcus cremoris subsp cremoris), yogurt (Lactobacillus delbrueckii subsp. bulgaricus, Streptococcus thermophilus), Swiss cheese (S. thermophilus, Lactobacillus lactis, Lactobacillus helveticus), Blue cheese (Leuconostoc cremoris), Italian cheese (L. bulgaricus, S. thermophilus), Viili (Lactococcus cremoris, Lactococcus lactis subsp. lactis biovar diacetylactis, Leuconostoc cremoris), Yakult (Lacticaseibacillus casei), casein (Lactococcus cremoris), Natto (Bacillus subtilis var. natto), Wine (Leuconostoc oenos), Sake (Leuconostoc mesenteroides), Polymyxin (Bacillus polymyxa), Colistin (Bacillus colistrium), Bacitracin (Bacillus licheniformis), L-Glutamic acid (Brevibacterium lactofermentum, Microbacterium ammoniaphilum), and acetone and butanol (Colstridium acetobutylicum, Clostridium saccharoperbutylacetonicum).

In some embodiments, the target nucleic acid is a mobile genetic element. In some embodiments, the target nucleic acid is a transposable element or insertion sequence. In some embodiments, the target nucleic acid is an insertion sequence. In some embodiments, the target nucleic acid is a transposable element.

In some embodiments, the target nucleic acid is a plasmid. In some embodiments, the target nucleic acid is a region within the plasmid DNA, such as sequences within the plasmid's origin of replication.

In some embodiments, the target nucleic acid is an undesirable genetic element. In some embodiments, removal of the undesirable genetic element results in a desirable bacterial phenotype. For example, a phenotype useful in food production, food protective, or probiotic cultures.

In some embodiments, the target nucleic acid is or is derived from a gene that is or is associated with resistance to antibiotics. By “antibiotic” is understood a chemical composition or moiety which decreases the viability or which inhibits the growth or reproduction of microbes. Antibiotic resistance genes include, but are not limited to tetracyclines (tet), chloramphenicol (cat), aminoglycosides (e.g., streptomycin), erythromycin (MLS—e.g., erm) and glycopeptides (e.g., transferrable vancomycin [van] resistance), blutₑm, blaᵣₒb, blaₛhv aadB, aacC1, aacC2, aacC3, aacA4, mecA, vanA, vanH, vanX, satA, aacA-aphH, vat, vga, msrA sul, and/or int. The antibiotic resistance genes include those that are or are derivable (preferably, derived) from bacterial species that include but are not limited to the genera Escherichia, Klebsiella, Pseudomonas, Proteus, Streptococcus, Staphylococcus, Enterococcus, Haemophilus and Moraxella. The antibiotic resistance genes also include those that are or are derivable (preferably, derived) from bacterial species that include but are not limited to Escherichia coli, Klebsiella pneumoniae, Pseudomonas aeruginosa, Proteus mirabilis, Streptococcus pneumoniae. Staphylococcus aureus, Staphylococcus epidermidis, Enterococcus faecalis, Staphylococcus saprophyticus, Streptococcus pyogenes, Haemophilus influenzae, and Moraxella catarrhalis. In some embodiments, the target nucleic acid is an antibiotic resistance encoding gene(s) that can prevent transfer of genes conferring resistance to antibiotics to cells, e.g., bacteria, thus reducing the risk of acquiring antibiotic resistance. By way of example, target nucleic acids may include vanR, (a gene conferring resistance to vancomycin), or tetR, a gene conferring resistance to tetracycline, or targeting beta-lactamase inhibitors. In some embodiments, the target nucleic acid is an antibiotic resistance gene.

In some embodiments, the target nucleic acid is a virulence factor. In some embodiments, the target nucleic acid may be or may be derived from a gene that is or is associated with genes encoding virulence factors. For example, factors commonly contributing to virulence in microbial pathogens can be targeted, such as toxins, internalins, and hemolysins. In some embodiments, the virulence factor is selected from the group consisting of a toxin-, an internalin- and a hemolysin-encoding nucleic acid.

In some embodiments, the target nucleic acid is a pathogenicity island.

In some embodiments, the target nucleic as defined herein is able to generate a CRISPR-Cas system-mediated response, e.g., maturation and interference. In some embodiments, the target nucleic as defined herein is able to generate a type III-A CRISPR-Cas system-mediated response.

### D. Bacterial Compositions

Further provided herein are bacterial strains, e.g., bacterial cells, containing any of the compositions described in Sections IA-IC. In some embodiments, the bacterial strain is a strain useful in food production, e.g., in the production of fermented food, such as a starter culture. In some embodiments, the bacterial strain is a strain useful in food protection, e.g., a protective culture. In some embodiments, the bacterial strain is a strain useful as a probiotic, optionally in a functional food or a dietary supplement.

In some embodiments, the bacterial strain is a Gram-positive bacterial strain. In some embodiments, the bacterial strain is a lactic acid bacterium. In some embodiments, the bacterial strain is a Bifidobacterium species, a Brevibacterium species, a Propionibacterium species, a Lactococcus species, a Streptococcus species, a Lactobacillus species, a Lactiplantibacillus species, a Lacticaseibacillus species, a Limosilactobacillus species, an Enterococcus species, a Pediococcus species, a Leuconostoc species and an Oenococcus species. Suitable species include, but are not limited to Streptococcus thermophilus, Lactobacillus acidophilus, Bifidobacterium lactis, Limosilactobacillus fermentum, Lacticaseibacillus casei, Lacticaseibacillus paracasei, Lacticaseibacillus rhamnosus, Lactiplantibacillus plantarum, Lactobacillus delbrueckii subsp bulgaricus, Propionibacteria freudenreichii, Pediococcus acidilactici, an Enterococcus faecium, a Lactococcus lactis, or a Lactococcus cremoris. In some embodiments, the bacterial strain is a Lactococcus lactis, a Lactococcus cremoris, or a biovar or subspecies thereof. In some embodiments, the bacterial strain is a Lactococcus lactis, a Lactococcus cremoris, or a biovar or subspecies thereof is a milk-adapted strain. In some embodiments, the bacterial strain is a Lactococcus lactis strain. In some embodiments, the bacterial strain is a Lactococcus lactis subsp lactis strain. In an embodiment, said bacterial strain is a Lactococcus cremoris strain. In an embodiment, said bacterial strain is a Lactococcus cremoris subsp cremoris strain.

In some embodiments, the bacterial strain contains a non-adapting CRISPR-Cas system as described in Section I-A. In some embodiments, the non-adapting CRISPR-Cas system is the native CRISPR-Cas system of the bacterial strain. In some embodiments, the non-adapting CRISPR-Cas system is present in a bacterial strain that does not or is not known to contain a non-adapting CRISPR-Cas system. In some embodiments, the bacterial strain containing the non-adapting CRISPR-Cas system is a recipient bacterial strain. In some embodiments, the bacterial strain, optionally a recipient bacterial strain, contains a non-adapting CRISPR-Cas system as described in Section I-A and compositions for enabling adaptation in non-adapting CRISPR-Cas systems, such as described in Section I-B.

In some embodiments, the bacterial strain, optionally a recipient bacterial strain, contains compositions for enabling adaptation in non-adapting CRISPR-Cas systems, such as described in Section I-B.

In some embodiments, the bacterial strain contains an adapted non-adapting CRISPR-Cas system as described in Section I-C. In some embodiments, the bacterial strain containing the adapted non-adapting CRISPR-Cas system is not the same bacterial strain that underwent methods of enabling adaptation in non-adapting CRISPR-Cas systems as described herein. Thus, in some embodiments, the bacterial strain containing the adapted non-adapting CRISPR-Cas system is a recipient bacterial strain. In this case, the adapted non-adapting CRISPR-Cas system is introduced to the recipient bacterial strain from the bacterial strain that underwent methods of enabling adaptation in non-adapting CRISPR-Cas systems as described herein.

In some embodiments, a recipient bacterial strain is from the same genus as the bacterial strain from which it received the adapted non-adapting CRISPR-Cas system. In some embodiments, the recipient bacterial strain is the same species as the bacterial strain from which it received the adapted non-adapting CRISPR-Cas system. In some embodiments, the recipient bacterial strain is the same strain as the bacterial strain from which it received the adapted non-adapting CRISPR-Cas system. In some embodiments, the recipient bacterial strain is from a different genus than the bacterial strain from which it received the adapted non-adapting CRISPR-Cas system. In some embodiments, the recipient bacterial strain is a different species than the bacterial strain from which it received the adapted non-adapting CRISPR-Cas system. In some embodiments, the recipient bacterial strain is a different strain than the bacterial strain from which it received the adapted non-adapting CRISPR-Cas system.

### II. METHODS OF ENABLING ADAPTATION

Provided herein are methods for enabling adaptation in non-adapting CRISPR-Cas systems, said methods including the compositions as described herein (see, Section I). In some embodiments, the methods provided herein produce adapted non-adapting CRISPR-Cas systems for example as described in Section I-C above. In some cases, the methods provided herein produce bacterial strains, for example as described in Section I-D, containing adapted non-adapting CRISPR-Cas systems. In some embodiments, the presence of the adapted non-adapting CRISPR-Cas system in the bacterial strain endows the bacterial strain with a resistance and/or a desirable phenotype.

The methods provided herein include combining non-adapting CRISPR-Cas systems and compositions for enabling adaptation in non-adapting CRISPR-Cas systems to allow natural spacer acquisition to occur in the CRISPR array of the non-adapting CRISPR-Cas system. In some cases, the combining occurs in vitro. For example, the non-adapting CRISPR-Cas system and composition for enabling adaptation may be combined in a cell-free system, e.g., an environment or media that is not in a cell. In this case, enabling spacer acquisition would occur in the cell-free system, for example, by adding target nucleic acids to the system. In this case, the methods described herein occur in a cell-free environment or system. In some embodiments, the combining occurs in vivo, for example in a cell. In some embodiments, the combining occurs in a bacterium. In some embodiments, the bacterium may be a species or strain of bacteria that naturally contains a non-adapting CRISPR-Cas system. In some embodiments, the bacterium may be a strain that does not or is not known to contain a non-adapting CRISPR-Cas system. In these cases, a non-adapting CRISPR-Cas system may be introduced into the bacterium, for example as described below.

In some embodiments, the method includes subjecting, e.g., exposing, the bacterium containing the non-adapting CRISPR-Cas systems and compositions for enabling adaptation to conditions that promote spacer acquisition. For example, in some embodiments, the bacterium may be subjected to phage challenge, e.g., by one or more phages. In this way, the non-adapting CRISPR-Cas system may be adapted to include new spacers that confer resistance to the phage. In some embodiments, the bacterium may be subjected, e.g., exposed, to challenge with a foreign nucleic acid, such as a mobile genetic element (MGE) or plasmid. In some embodiments, the foreign nucleic acid, e.g., MGE, plasmid, may be an element that confers antibiotic resistance, a virulence factor, or provide for toxin production. In this way, the non-adapting CRISPR-Cas system may be adapted to include new spacers that confer resistance to the foreign nucleic acid, e.g., MGE, plasmid. In some embodiments, the bacterium may be subjected, e.g., exposed, to stresses or selective pressures that depend on a desirable phenotype. In this way, the non-adapting CRISPR-Cas system may be adapted to include new spacers that confer a desirable phenotype to the bacterium. In some embodiments, the bacterium may be subjected, e.g., exposed, to one or more conditions to that promote new spacer acquisition. Thus, in some aspects, the non-adapting CRISPR-Cas system will be adapted, e.g., acquire new spacers, such that the adapted non-adapting CRISPR-Cas system includes a known immune profile. In some embodiments, the known immune profile is customized to produce bacterial strains with known immune profiles. In some embodiments, a plurality of known immune profiles may be generated to produce a plurality of bacterial strains with known immune profiles. In some embodiments, the plurality of bacterial strains with known immune profiles may be used to ensure the success of bacterial cultures for their intended purpose, for example as described in Section III.

In some embodiments, the method further includes curing the bacterial strain which has undergone the methods of enabling adaptation as described herein to remove the compositions for enabling adaptation, for example as described in Sections I-B1 to B3.

In some embodiments, the method further includes introducing the adapted non-adapting CRISPR-Cas system (see, e.g., Section I-C) to a recipient bacterial strain. In some embodiments, the recipient bacterial strain is as described in Section I-C.

The methods described herein are contemplated to include any or all of the steps described herein.

A. Introduction of Compositions for Enabling Adaptation into Bacterial Strains

The compositions described herein in Sections I-B1 to I-B3 for enabling adaptation in such systems, may be introduced into a bacterial strain using any method available.

“Introducing,” “introduced,” and grammatical variants thereof are intended to mean presenting to the bacterial strain polynucleotides, constructs, vectors, plasmids, and polypeptides for enabling adaptation as defined herein, in such a manner that the component(s) gains access to the interior of a bacterium. The methods and compositions do not depend on a particular method for introducing compositions for enabling adaptation into a bacterial strain, only that the composition gains access to the interior of the bacterium. In some embodiments, the introducing includes the incorporation of a nucleic acid sequence or polynucleotide into the bacterial strain where the nucleic acid or polynucleotide is incorporated into the genome of the bacterial strain and includes the transient (direct) provision of a nucleic acid sequence or polynucleotide or protein to the host cell. In some embodiments, the introducing includes the incorporation of a nucleic acid or polynucleotide into the bacterial strain where the nucleic acid sequence or polynucleotide is not incorporated into the genome of the bacterial strain. In some embodiments, the compositions, e.g., nucleic acids, polynucleotides, are not incorporate into the bacterial strain genome.

In some embodiments, introducing a nucleic acid sequence, construct, vector, or polypeptide into a strain can be carried out by several methods, including transformation, conjugation, transduction, or protoplast fusion. Methods for introducing polynucleotides or polypeptides by transformation into a host cell, include, but are not limited to, microinjection, electroporation, stable transformation methods, transient transformation methods (such as induced competence using chemical (e.g. divalent cations such as CaCl₂), mechanical (electroporation) means, or methods such as those described in published international applications WO 2018/114983 and WO 2010/149721, which are incorporated herein by reference in their entireties), ballistic particle acceleration (particle bombardment), direct gene transfer, viral-mediated introduction, cell-penetrating peptides, or mesoporous silica nanoparticle (MSN)-mediated direct protein delivery. Introducing a nucleic acid, construct, plasmid, or vector into a strain can be carried out by conjugation, which is a specific method of natural DNA exchange requiring physical cell-to-cell contact. Introducing a nucleic acid, construct, plasmid, or vector into a strain can be carried out by transduction, which is the introduction of DNA via a virus (e.g. phage) infection which is also a natural method of DNA exchange. Generally, such methods involve incorporating a polynucleotide within a viral DNA or RNA molecule.

A protein, such as a Cas polypeptide described herein, can be introduced into a host cell by directly introducing the protein itself or an mRNA encoding the protein. The protein can be introduced into a host cell transiently. Uptake of the protein into the host cell can be facilitated with a Cell Penetrating Peptide (CPP).

In some embodiments, the introduction is stable, i.e., that the nucleic acid (construct, plasmid, or vector) introduced into the bacterial strain integrates into a genome of the host cell and is capable of being inherited by the progeny thereof. In another embodiment, the introduction can be temporary, i.e., that a nucleic acid (construct, plasmid, or vector) is introduced into the bacterial strain and does not integrate into a genome of the host cell or a polypeptide is introduced into the bacterial strain. Transient transformation indicates that the introduced nucleic acid or protein is only temporarily expressed or present in the bacterial strain. In some embodiments, transient introduction enables curing of the composition from the cell.

In some embodiments, the bacterial strain into which the composition for enabling adaptation in non-adapting CRISPR-Cas systems is introduced is a bacterial strain that contains a non-adapting CRISPR-Cas system, such as a non-adapting CRISPR-Cas system as described in Section I-A. In some embodiments, the non-adapting CRISPR-Cas system is the native CRISPR-Cas system of the bacterial strain. In some embodiments, the bacterial strain containing the non-adapting CRISPR-Cas system is does not or is not known, e.g., under naturally occurring conditions, to contain a non-adapting CRISPR-Cas system. In some embodiments, the bacterial strain containing the non-adapting CRISPR-Cas system is a recipient bacterial strain. Thus, in some embodiments, the bacterial strain containing the adapted non-adapting CRISPR-Cas system is a recipient bacterial strain. In some embodiments, the bacterial strain or recipient bacterial strain is a bacterium of any one or more of the genus species or strains described in Section I-D or Section III-A below.

In some embodiments, for example when the non-adapting CRISPR-Cas system is present in a recipient bacterial strain, the non-adapting CRISPR-Cas system is introduced according to the any of the methods of introducing a nucleic acid sequence, polynucleotide, vector, or plasmid as described herein, e.g., supra.

In some embodiments, a polynucleotide is introduced into the bacterial strain. In some embodiments, the polynucleotide is or includes a nucleic acid sequence encoding a Cas1 polypeptide as disclosed in Section I-B1. In some embodiments, the polynucleotide is or includes a nucleic acid sequence encoding a Cas2 polypeptide as disclosed in Section I-B1. In some embodiments, the polynucleotide further includes a heterologous regulatory sequence operably linked to the nucleic acid sequence encoding the Cas1 or Cas2 polypeptide.

In some embodiments, a first polynucleotide is introduced into the bacterial strain. In some embodiments, the first polynucleotide is or includes a nucleic acid sequence encoding a Cas1 polypeptide as disclosed in Section I-B1. In some embodiments, a second polynucleotide is or includes a nucleic acid sequence encoding a Cas2 polypeptide as disclosed in Section I-1. In some embodiments, the first polynucleotide further includes a heterologous regulatory sequence operably linked, directly or indirectly, to the nucleic acid sequence encoding the Cas1. In some embodiments, the second polynucleotide further includes a heterologous regulatory sequence operably linked, directly or indirectly, to the nucleic acid sequence encoding the Cas2.

In some embodiments, a polynucleotide including a nucleic acid sequence encoding a Cas1 polypeptide and a Cas2 polypeptide, as described in Section I-B, is introduced to the bacterial strain. In some embodiments, the polynucleotide further includes a first heterologous regulatory sequence operably linked, directly or indirectly, to the nucleic acid sequence encoding the Cas1 polypeptide. In some embodiments, the polynucleotide includes a second heterologous regulatory sequence operably linked, directly or indirectly, to the nucleic acid sequence encoding the Cas2 polypeptide. In some embodiments, when two heterologous regulatory sequences are present in the polynucleotide, the heterologous regulatory sequences are first and second heterologous regulatory sequences.

In some embodiments, a polynucleotide, e.g., construct, including from 5′ to 3′ a first heterologous regulatory sequence, a nucleic acid sequence encoding a Cas1 polypeptide, a second heterologous regulatory sequence, and a Cas2 polypeptide, as described in Section I-B, is introduced to the bacterial strain. In some embodiments, a polynucleotide, e.g., construct, including from 5′ to 3′ a first heterologous regulatory sequence, a nucleic acid sequence encoding a Cas2 polypeptide, a second heterologous regulatory sequence, and a Cas1 polypeptide, as described in Section I-B, is introduced to the bacterial strain. In some embodiments, a polynucleotide, e.g., construct, including from 5′ to 3′ a heterologous regulatory sequence, a nucleic acid sequence encoding a Cas1 polypeptide, a polycistronic sequence, and a Cas2 polypeptide, as described in Section I-B, is introduced to the bacterial strain. In some embodiments, a polynucleotide, e.g., construct, including from 5′ to 3′ a heterologous regulatory sequence, a nucleic acid sequence encoding a Cas2 polypeptide, a polycistronic sequence, and a Cas1 polypeptide, as described in Section I-B, is introduced to the bacterial strain.

In some embodiments, a vector, e.g., plasmid, is introduced into the bacterial strain. In some embodiments, the vector, e.g., plasmid, is or includes a nucleic acid sequence encoding a Cas1 polypeptide as disclosed in Section I-B1. In some embodiments, the vector, e.g., plasmid, is or includes a nucleic acid sequence encoding a Cas2 polypeptide as disclosed in Section I-B1. In some embodiments, the vector, e.g., plasmid, further includes a heterologous regulatory sequence operably linked to the nucleic acid sequence encoding the Cas1 or Cas2 polypeptide.

In some embodiments, a first vector and a second vector are introduced into the bacterial strain. In some embodiments, the first vector, e.g., plasmid, is or includes a nucleic acid sequence encoding a Cas1 polypeptide as disclosed in Section I-B1. In some embodiments, the second vector, e.g., plasmid, is or includes a nucleic acid sequence encoding a Cas2 polypeptide as disclosed in Section I-B1. In some embodiments, the first vector, e.g., plasmid, further includes a heterologous regulatory sequence operably linked, directly or indirectly, to the nucleic acid sequence encoding the Cas1. In some embodiments, the second vector, e.g., plasmid, further includes a heterologous regulatory sequence operably linked, directly or indirectly, to the nucleic acid sequence encoding the Cas2.

In some embodiments, a vector, e.g., plasmid, including a nucleic acid sequence encoding a Cas1 polypeptide and a Cas2 polypeptide, as described in Section I-B, is introduced to the bacterial strain. In some embodiments, the vector, e.g., plasmid, further includes a first heterologous regulatory sequence operably linked, directly or indirectly, to the nucleic acid sequence encoding the Cas1 polypeptide. In some embodiments, the vector, e.g., plasmid, includes a second heterologous regulatory sequence operably linked, directly or indirectly, to the nucleic acid sequence encoding the Cas2 polypeptide.

In some embodiments, a vector, e.g., plasmid, including from 5′ to 3′ a first heterologous regulatory sequence, a nucleic acid sequence encoding a Cas1 polypeptide, a second heterologous regulatory sequence, and a Cas2 polypeptide, as described in Section I-B, is introduced to the bacterial strain. In some embodiments, a vector, e.g., plasmid, including from 5′ to 3′ a first heterologous regulatory sequence, a nucleic acid sequence encoding a Cas2 polypeptide, a second heterologous regulatory sequence, and a Cas1 polypeptide, as described in Section I-B, is introduced to the bacterial strain. In some embodiments, a vector, e.g., plasmid, including from 5′ to 3′ a heterologous regulatory sequence, a nucleic acid sequence encoding a Cas1 polypeptide, a polycistronic sequence, and a Cas2 polypeptide, as described in Section I-B, is introduced to the bacterial strain. In some embodiments vector, e.g., plasmid, including from 5′ to 3′ a heterologous regulatory sequence, a nucleic acid sequence encoding a Cas2 polypeptide, a polycistronic sequence, and a Cas1 polypeptide, as described in Section I-B, is introduced to the bacterial strain.

It should be appreciated that in some cases, for example when the compositions for enabling adaptation are under the control of an inducible promoter, methods for inducing spacer acquisition (see, e.g., Section II-B) occur under conditions appropriate to allow expression. Exemplary conditions are further described herein (see, e.g., Section I-B1).

In some embodiments, one or more polypeptides as described in Section I-B3 are introduced into the bacterial strain. In some embodiments, the polypeptide is or includes a Cas1 polypeptide as described herein (see, e.g., Section I-B3). In some embodiments, the polypeptide is or includes a Cas2 polypeptide as described herein (see, e.g., Section I-B3). In some embodiments, the polypeptide is or includes a Cas1 and a Cas2 polypeptide as described herein (see, e.g., Section I-B3). In some embodiments, the Cas1 and Cas2 polypeptides are contained in a single amino acid sequence. In this case, the use of spacers and/or linkers may be used to ensure that the proteins fold and/or interact to exhibit functional behavior (e.g., adaptation). In some embodiments, the Cas1 and Cas2 polypeptides are introduced to the bacterial strain as separate polypeptides. In some embodiments, the Cas1 and Cas2 polypeptides are introduced to the bacterial strain as a single polypeptide, optionally including spacers or linkers to retain functional activity.

### B. Inducing Spacer Acquisition

In some aspects, the methods provided herein allow acquisition of new spacer sequences in CRISPR arrays of non-adapting CRISPR-Cas systems. The resulting non-adapting CRISPR-Cas systems containing new spacers are referred to herein as adapted non-adapting CRISPR-Cas systems as described in Section I-C.

It is contemplated that new spacers may be acquired from a variety of nucleic acid sources, referred to herein as target nucleic acids. Non-limiting examples of target nucleic acids include a bacteriophage genome, a transcription product of a bacteriophage genome, a plasmid, a resident plasmid, a chromosomal sequence, a mobile genetic element (MGEs), a transposable element, or an insertion sequence. Target nucleic acids considered according to the methods provided herein are further described in Section I-C above.

In some embodiments, the acquisition of new spacers according to the methods provided herein confers resistance against one or more bacteriophages. In some embodiments, the acquisition of new spacers according to the methods provided herein confers resistance against one or more plasmids. In some embodiments, the acquisition of new spacers according to the methods provided herein confers resistance against one or more MGEs. In some embodiments, the acquisition of new spacers according to the methods provided herein confers resistance against one or more transposable elements. In some embodiments, the acquisition of new spacers according to the methods provided herein confers resistance against one or more insertion sequences. In some embodiments, the acquisition of new spacers according to the methods provided herein confers a desirable phenotype, e.g., by the presence of a self-targeting spacer.

In some cases, the new spacers acquired by the CRISPR array are not limited to one type of resistance, e.g., plasmid, MGE, bacteriophage, or desirable phenotype, e.g., self-targeting spacer. In some embodiments, the methods provided herein allow for multiple spacers directed to different target nucleic acids to be acquired. For example, a bacterial cell containing a non-adapting CRISPR-Cas system and compositions for enabling adaptation as described herein may be exposed to one or more or a plurality of target nucleic acids, e.g., different target nucleic acids, or environmental conditions, e.g., selective pressures, stressors, such that the new spacers acquired confer one or more or a plurality of resistances and desirable phenotypes. Thus, the methods provided herein should be viewed as optionally used in combination to produce an adapted non-adapting CRISPR-Cas system customized to provide resistances and desirable phenotypes. The methods provided herein may be used to produce an immune profile as required for a particular purpose. In some cases, the immune profile may be customized to produce bacterial strains for food productions, e.g., in the production of fermented food, such as a starter culture. In some embodiments, the immune profile may be customized to produce bacterial strains for food protection, e.g., a protective culture. In some embodiments, the immune profile may be customized to produce bacterial strains for probiotics, optionally in a functional food or a dietary supplement.

### 1. Plasmids and MGEs

In an aspect is provided a method for producing bacterial strain resistant to nucleic acid. In some embodiments, the method includes exposing a bacterial strain containing a non-adapting CRISPR-Cas system and a composition for enabling adaptation in a non-adapting CRISPR-Cas system as described in, e.g., Section I-A, to a target nucleic acid. In some embodiments, exposing refers to contacting, e.g., by mixing, the bacterial strain with the target nucleic acid so as to induce adaptation. In some embodiments, the target nucleic acid is foreign nucleic acid. As used herein, a foreign nucleic acid refers to a nucleic acid that is not present in the genome or a plasmid, or transcripts thereof, resident in the bacterial strain exposed or to be exposed to the target nucleic acid. In some embodiments, the target nucleic acid is a foreign DNA. In some embodiments, the target nucleic acid is a foreign RNA. In some embodiments, the target nucleic acid, e.g., foreign nucleic acid, is a plasmid. In some embodiments, the target nucleic acid, e.g., foreign nucleic acid, is a mobile genetic element. In some embodiments, the mobile genetic element is a transposable element. In some embodiments, the mobile genetic element is an insertion sequence. In some embodiments, the foreign nucleic acid encodes an antibiotic resistance. In some embodiments, the foreign nucleic acid is an antibiotic resistance gene. In some embodiments, the foreign nucleic acid encodes a virulence factor. In some embodiments, the foreign nucleic acid is an antibiotic resistance gene. In some embodiments, the foreign nucleic acid encodes a toxin. In some embodiments, the foreign nucleic acid is a toxin gene.

In some embodiments, the method produces bacterial strains resistant to acquiring a plasmid or MGE.

In some embodiments, the bacterial strain is exposed to one or more target nucleic acids, e.g., different target nucleic acids as described herein. In some embodiments, the bacterial strain is exposed to 1, 2, 3, 4, 5, 6, 7, 8, 9, 10, or more target nucleic acids, e.g., different target nucleic acids. In some embodiments, the bacterial strain is exposed to about 1 to about 10 target nucleic acids, e.g., different target nucleic acids. In some embodiments, the bacterial strain is exposed to about 1 to about 5 target nucleic acids, e.g., different target nucleic acids. In some embodiments, the bacterial strain is exposed to about 1 to about 4 target nucleic acids, e.g., different target nucleic acids. In some embodiments, the bacterial strain is exposed to about 1 to about 3 target nucleic acids, e.g., different target nucleic acids. In some embodiments, the bacterial strain is exposed to about 1 to about 2 target nucleic acids, e.g., different target nucleic acids. In some embodiments, the bacterial strain is exposed to 1 target nucleic acid. In some embodiments, the bacterial strain is exposed to 2 target nucleic acids. In some embodiments, the bacterial strain is exposed to 3 target nucleic acids. In some embodiments, the bacterial strain is exposed to 4 target nucleic acids. In some embodiments, the bacterial strain is exposed to 5 target nucleic acids. In some embodiments, when the bacterial strain is exposed to more than one target nucleic acid, e.g., different target nucleic acids, the exposure occurs simultaneously. For example, the bacterial strain is exposed, e.g., contacted, with each target nucleic acid at the same time or at a temporally proximal time, e.g., added to a medium containing the bacterial strain one after the other, and the bacterial strain is not assessed for resistance to a target nucleic acid prior to addition of another target nucleic acid sequence. In some embodiments, when the bacterial strain is exposed to more than one target nucleic acid, e.g., different target nucleic acids, the exposure occurs sequentially. For example, the bacterial strain is exposed, e.g., contacted, with a target nucleic acid after a duration of time has elapsed, e.g., a duration of time where the probability of at least one bacterium acquiring a spacer is at least 50%, e.g., 60%, 70%, 80%, 90%, 95%, or more. In some embodiments, when the target nucleic acids are added sequentially, the exposed bacterial strain may be assessed for resistance, e.g., by challenge or new spacer identification (e.g., sequencing and/or comparison), to the previously exposed target nucleic acid prior to addition of another target nucleic acid. In some embodiments, only bacterial strains with confirmed resistance to the target nucleic acid are exposed to a further target nucleic acid.

### 2. Bacteriophages

In an aspect is provided a method for producing bacterial strain resistant to bacteriophages. In some embodiments, the method includes exposing a bacterial strain containing a non-adapting CRISPR-Cas system and a composition for enabling adaptation in a non-adapting CRISPR-Cas system as described in, e.g., Section I-A, to a bacteriophage. In some embodiments, exposing refers to contacting, e.g., by mixing, the bacterial strain with the bacteriophage so as to induce adaptation. In some embodiments, the bacteriophage is a bacteriophage known or suspected of infecting bacterial strains used for food production. In some embodiments, the bacteriophage is a bacteriophage known or suspected of infecting bacterial strains used for food protection. In some embodiments, the bacteriophage is a bacteriophage known or suspected of infecting bacterial strains used for probiotics. In some embodiments, the bacteriophage is a bacteriophage commonly found in industrial setting, such as food processing plants. In some embodiments, the bacteriophage is a newly identified bacteriophage. For example, the newly identified bacteriophage may be bacteriophage newly identified in an industrial setting, e.g., a food processing plant. In some embodiments, the bacteriophage may be a bacteriophage that the bacterial strain is known not to have resistance to.

In some embodiments, the bacterial strain is exposed to one or more bacteriophages, e.g., different bacteriophages as described herein. In some embodiments, the bacterial strain is exposed to 1, 2, 3, 4, 5, 6, 7, 8, 9, 10, or more bacteriophages, e.g., different bacteriophages. In some embodiments, the bacterial strain is exposed to about 1 to about 10 bacteriophages, e.g., different bacteriophages. In some embodiments, the bacterial strain is exposed to about 1 to about 5 bacteriophages, e.g., different bacteriophages. In some embodiments, the bacterial strain is exposed to about 1 to about 4 bacteriophages, e.g., different bacteriophages. In some embodiments, the bacterial strain is exposed to about 1 to about 3 bacteriophages, e.g., different bacteriophages. In some embodiments, the bacterial strain is exposed to about 1 to about 2 bacteriophages, e.g., different bacteriophages. In some embodiments, the bacterial strain is exposed to 1 bacteriophage. In some embodiments, the bacterial strain is exposed to 2 bacteriophages. In some embodiments, the bacterial strain is exposed to 3 bacteriophages. In some embodiments, the bacterial strain is exposed to 4 bacteriophages. In some embodiments, the bacterial strain is exposed to 5 bacteriophages. In some embodiments, when the bacterial strain is exposed to more than one bacteriophage, e.g., different bacteriophages, the exposure occurs simultaneously. For example, the bacterial strain is exposed, e.g., contacted, with each bacteriophage at the same time or at a temporally proximal time, e.g., added to a medium containing the bacterial strain one after the other, and the bacterial strain is not assessed for resistance to a bacteriophage prior to addition of another bacteriophage sequence. In some embodiments, when the bacterial strain is exposed to more than one bacteriophage, e.g., different bacteriophages, the exposure occurs sequentially. For example, the bacterial strain is exposed, e.g., contacted, with a bacteriophage after a duration of time has elapsed, e.g., a duration of time where the probability of at least one bacterium acquiring a spacer is at least 50%, e.g., 60%, 70,%, 80%, 90%, 95%, or more. In some embodiments, when the bacteriophages are added sequentially, the exposed bacterial strain may be assessed for resistance, e.g., by challenge or new spacer identification (e.g., sequencing and/or comparison), to the previously exposed bacteriophage prior to addition of another bacteriophage. In some embodiments, only bacterial strains with confirmed resistance to the bacteriophage are exposed to a further bacteriophage.

### 3. Desirable Phenotypes

In an aspect is provided a method for producing an evolved bacterial strain, where the bacterial strain exhibits a desirable phenotype. In some embodiments, the method includes exposing a bacterial strain containing a non-adapting CRISPR-Cas system and a composition for enabling adaptation in a non-adapting CRISPR-Cas system as described in, e.g., Section I-A, to stresses or selective pressures designed to promote a desirable phenotype. In this case, exposing refers to incubating the bacterial strain under culture conditions dependent on the desirable phenotype. In some embodiments, the exposure promotes proliferation of a bacterial strain having a desirable phenotype. The choice of selective pressure or stressor can be selected according to the desirable phenotype to be achieved.

Stressors and selective pressures contemplated herein include, but are not limited to, environment variables that impact bacterial fitness. For example, in some cases, the stressors and selective pressures lead to a decrease in bacterial growth rate or competitive ability. Under stressors and selective pressures, only bacteria with a particular phenotype(s) may be able to survive and growth. Various selective pressure or stressor to achieve a desirable phenotype are known in the art.

In some embodiments, the stressor or selective pressure is starvation. In some embodiments, starvation is induced by the normal depletion of nutrients during batch culture. In some embodiments, starvation is induced by suspending bacteria in a nutrient-free medium.

In some embodiments, the stressor or selective pressure is a nutrient-limitation. In some embodiments, nutritional-limitation includes growing bacteria in a media lacking one nutrient. In some embodiments, more than one nutrient, e.g., 2, 3, 4, 5, or more, nutrients are not present, but at least one nutrient is present.

In some embodiments, the stressor or selective pressure is a nutritional selection. In this case, a limitation of a nutrient may be overcome by mutation. For example, a nutritional selection may be induced by growing a bacteria on an energy source or carbon source which it does not or is not known to metabolize. By way of example, nutritional selection may be induced by incubating bacteria incapable of metabolizing lactose with lactose as the only energy or carbon source.

In some embodiments, the stressor or selective pressure is hunger. In some embodiments, hunger is induced by growing bacteria in the presence of suboptimal levels of nutrients. By way of example, hunger may be induced by growing bacteria in nutrient-limited chemostats.

In some embodiments, the stressor or selective pressure is temperature selection. In some embodiments, temperature selection is induced by temperature changes, such as rapid increases or decreases in temperature. In some embodiments, temperature selection is induced by heating the bacteria to about 42 to about 47° C., e.g., heat-shock treatment. In some embodiments, temperature selection is induced by cooling the bacteria to between about 0 to 15° C., e.g., cold shock treatment. In some embodiments, the change in temperature occurs within less than 10, 5, 4, 3, 2, or 1 minute or less. In some embodiments, the changed temperature is sustained for at least 5 minute, 10 minutes, 20 minutes, 30 minutes, 1 hour, 2 hours, 3 hours, 5 hours, 10 hours, 24 hours, or more.

In some embodiments, the stressor or selective pressure is oxidative stress. In some embodiments, the stressor or selective pressure is alkylation stress. In some embodiments, the stressor or selective pressure is a pH. In some embodiments, the pH is a low pH, such as a pH below 6.0, 5.0, 4.0, 3.0, 2.0, or 1.0. In some embodiments, the low pH is a pH between about 1.0 and 5.5. In some embodiments, the stressor or selective pressure is osmotic pressure. In some embodiments, osmotic pressure includes an increase or a decrease in osmotic pressure. In some embodiments, osmotic pressure, either an increase or decrease in osmotic pressure, may be induced by incubating the bacteria in media containing different amounts of salt (e.g., NaCl) or sucrose.

In some embodiments, the stressor or selective pressure is a growth rate reduction. In some embodiments, the stressor or selective pressure is a toxin. For example, the bacteria may be grown in the presence of one or more toxins. In some embodiments, the stressor or selective pressure is an antibiotic. For example, the bacteria may be grown in the presence of one or more antibiotics.

In some embodiments, the exposure to the stressor or selective pressure results in the acquisition of self-targeting spacer sequences. For example, the spacers may be targeted against the bacterial strain genome or resident plasmids and/or transcripts thereof. In some embodiments, the self-targeting spacer sequences are active against nucleic acids that endow the bacterial strain with undesirable phenotype, their removal thereby conferring a desirable phenotype. In some embodiments, the undesirable phenotype is an antibiotic resistance. In some embodiments, the undesirable phenotype is a virulence factor. In some embodiments, the undesirable phenotype is a toxin production. In some embodiments, the undesirable phenotype is a biogenic amine. In some embodiments, the undesirable phenotype is a bacteriocin.

In some embodiments, the self-targeting spacer may alter expression of a desirable or undesirable phenotype, thereby conferring a desirable phenotype. Non-limiting examples of altering expression of desirable or undesirable phenotypes include altering a promoter, a repressor, an activator, a two-component regulatory system, or quorum sensing is altered.

In some embodiments, the self-targeting spacer may alter the behavior, e.g., inactive, an enzyme of the bacterial strain, thereby conferring a desirable phenotype. Non-limiting examples of enzyme modification capable of conferring a desirable phenotype include: the inactivation of any glycosyl transferases in the eps operon, which could abrogate production of exopolysaccharide, alter the composition of exopolysaccharides, e.g., compared to the bacterial strain not containing a self-targeting spacer sequence, or change, e.g., compared to a bacterial strain not containing a self-targeting spacer sequence, an amount of exopolysaccharides produced; the inactivation of d-lactose dehydrogenase, which could eliminate production of d-lactate; the inactivation of acetolactate decarboxylase, which could promote accumulation of acetolactate for conversion to diacetyl.

In some embodiments, the desirable phenotype is a phenotype useful in food production. In some embodiments, the desirable phenotype is a phenotype useful in food protective. In some embodiments, the desirable phenotype is a phenotype useful for probiotics. For example, the phenotype may be useful in for the cultures and products described in Section III.

In some embodiments, the bacterial strain is exposed to one or more selective pressures or stressors, e.g., different selective pressures or stressors as described herein. In some embodiments, the bacterial strain is exposed to 1, 2, 3, 4, 5, 6, 7, 8, 9, 10, or more selective pressures or stressors, e.g., different selective pressures or stressors. In some embodiments, the bacterial strain is exposed to about 1 to about 10 selective pressures or stressors, e.g., different selective pressures or stressors. In some embodiments, the bacterial strain is exposed to about 1 to about 5 selective pressures or stressors, e.g., different selective pressures or stressors. In some embodiments, the bacterial strain is exposed to about 1 to about 4 selective pressures or stressors, e.g., different selective pressures or stressors. In some embodiments, the bacterial strain is exposed to about 1 to about 3 selective pressures or stressors, e.g., different selective pressures or stressors. In some embodiments, the bacterial strain is exposed to about 1 to about 2 selective pressures or stressors, e.g., different selective pressures or stressors. In some embodiments, the bacterial strain is exposed to 1 selective pressures or stressor. In some embodiments, the bacterial strain is exposed to 2 selective pressures or stressors. In some embodiments, the bacterial strain is exposed to 3 selective pressures or stressors. In some embodiments, the bacterial strain is exposed to 4 selective pressures or stressors. In some embodiments, the bacterial strain is exposed to 5 selective pressures or stressors. In some embodiments, when the bacterial strain is exposed to more than one selective pressure or stressor, e.g., different selective pressures or stressors, the exposure occurs simultaneously. For example, the bacterial strain is exposed, e.g., contacted, with each selective pressure or stressor at the same time or at a temporally proximal time, e.g., added to a medium containing the bacterial strain one after the other, and the bacterial strain is not assessed for resistance to a selective pressure or stressor prior to addition of another selective pressures or stressor sequence. In some embodiments, when the bacterial strain is exposed to more than one selective pressure or stressor, e.g., different selective pressures or stressors, the exposure occurs sequentially. For example, the bacterial strain is exposed, e.g., contacted, with a selective pressure or stressor after a duration of time has elapsed, e.g., a duration of time where the probability of at least one bacterium acquiring a spacer is at least 50%, e.g., 60%, 70,%, 80%, 90%, 95%, or more. In some embodiments, when the selective pressures or stressors are added sequentially, the exposed bacterial strain may be assessed for resistance, e.g., by challenge or new spacer identification (e.g., sequencing and/or comparison), to the previously exposed selective pressure or stressor prior to addition of another selective pressure or stressor. In some embodiments, only bacterial strains with confirmed resistance to the selective pressure or stressor are exposed to a further selective pressures or stressor.

C. Bacteria with Adapted Non-Adapting CRISPR-Cas Systems

The methods provided herein may be used to produce bacterial strains with known, e.g., customized, adaptive immune profiles.

1. Selection and/or Isolation of Bacteria

In some embodiments, bacterial strains that have undergone methods for inducing spacer acquisition, e.g., as described in Section II-B above, and display expected resistances and/or desirable phenotypes are selected and/or isolated. In some embodiments, confirmation of an expected resistance and/or desirable phenotype is determined by testing the bacterial strain for resistances and/or desirable phenotypes. The methods of testing to confirm resistances and/or desirable attributes will depend on the types and combinations of exposures the bacterial strain was subjected to. Suitable methods of testing resistances, e.g., to phages, MGEs, plasmids, or the presence of a desirable attribute, e.g., exopolysaccharide or d-lactate production, are known in the art and may be selected for use accordingly.

In some embodiments, the bacterial strains displaying expected resistances and/or desirable phenotypes are selected. In some embodiments, the bacterial strains displaying expected resistances and/or desirable phenotypes are isolated, e.g., purified, from bacterial strains lacking expected resistances and/or desirable phenotypes and/or other bacterial strains with expected resistances and/or desirable phenotypes. It is contemplated that the methods provided herein may produce bacterial strains, e.g., cells, that may or may not be genetically identical, e.g., in terms of spacer sequences acquired, and/or exhibit the same resistance and/or desirable phenotype. By way of example, a bacterial strain having undergone phage challenge to induce spacer acquisition may contain a subset of cells having a more or less robust resistance to the phage compared to other cells of the bacterial strain, e.g., because the cells acquired different spacer sequences. Similarly, bacterial strains exposed to stressors or selective pressures may acquire different self-targeting spacers that result in differential expression of the same desirable phenotype. Thus, in some cases, bacterial strains displaying expected resistances and/or desirable phenotypes produced according the methods described herein may be selected and/or isolated from bacterial strains lacking expected resistances and/or desirable phenotypes. In some embodiments, bacterial strains displaying expected resistances and/or desirable phenotypes produced according the methods described herein may be selected and/or isolated from bacterial strains displaying expected resistances and/or desirable phenotypes but which express such characteristics to a different extent and/or have a different genotype. In some embodiments, the different extent, e.g., a strength of resistance, an expression of desirable phenotype, may be a difference from a mean resistance and/or a mean expression of a desirable phenotype of a bacterial strain. In some embodiments, the different expression of a resistance and/or desirable phenotype is identified as a 1, 2, 3 or more standard deviations from the mean. In some embodiments, the different expression of a resistance and/or desirable phenotype is identified as a positive or negative difference of more than of 10%, 20%, 30, 40%, 50%, 60%, 70%, 80%, or 90% from the mean. Numerous mathematical methods for assessing differences in data are known in the art and are contemplated for use herein. In some embodiments, a difference in genotype is determined by comparing the spacer sequences acquired by the cells of the bacterial strain. In some embodiments, spacer sequences with less than 99%, 98%, 97%, 96%, 95%, 94%, 93%, 92%, 91%, or 90% sequence identity are identified as a different genotype.

In some embodiments, the selected and or isolated bacterial strains are assessed to determine the presence of one or more new spacer sequences. In some embodiments, the one or more new spacer sequences are spacer sequences that are not present in the non-adapting CRISPR-Cas system (CRISPR array), e.g., prior to exposing the bacterial strain to induce spacer acquisition as described in Section II-B. In some embodiments, the spacer sequences of the non-adapting CRISPR-Cas system prior to inducing spacer acquisition are known. Thus, in some cases, only the spacer sequences in the bacterial strain exposed to spacer acquisition inducing conditions are determined. In some cases, for example when spacer sequences of the non-adapting CRISPR-Cas system are not known prior to exposure to spacer acquisition inducing conditions, spacer sequences of a non-adapting CRISPR-Cas system that is the same as the non-adapting CRISPR-Cas system that underwent spacer acquisition induction are determined in addition to the spacer sequences of the non-adapting CRISPR-Cas system that underwent spacer acquisition induction. In some embodiments, the spacer sequences identified in the adapted non-adapting CRISPR-Cas system and the non-adapting CRISPR-Cas system are compared.

In some embodiments, the presence of one or more spacer sequences is determined by PCR. In some embodiments, the presence of one or more spacer sequences is determined by sequencing methods. In some embodiments, PCR and sequencing are used to identify the presence of spacer sequences, e.g., new spacer sequences, in a CRISPR array. Exemplary methods of detecting new spacers include, but are not limited to, those described in Example 1 below and DNA sequencing.

### 2. Removal of Compositions for Enabling Adaptation

In some embodiments, the compositions for enabling adaptation in the non-adapting CRISPR-Cas system are removed, e.g., cured, from the bacterial strain that underwent the methods of inducing spacer acquisition as described in Section II-B. In some embodiments, the compositions for enabling adaptation in the non-adapting CRISPR-Cas system are removed from the selected and/or isolated bacterial strain. It should be appreciated that, in some cases, removal or curing of compositions for enabling adaptation is optional. For example, in some instances, the adapted non-adapting CRISPR-Cas system, or a portion thereof, may be introduced into a recipient bacterial strain. See, Section II-C3. In such cases, it may not be necessary to remove the compositions for enabling adaptation from the bacterial strain that has undergone adaptation according to Section II-B.

As described supra, in some embodiments, the compositions for enabling adaptation, e.g., vectors, are labile. Thus, in some cases, following the methods of inducing spacer acquisition as described in Section II-B, the bacterial strain is treated so as to destroy the compositions for enabling adaptation, e.g., vectors, present in the bacterial cells. In some embodiments, the compositions for enabling adaptation, e.g., vectors, are cured naturally through cell division. In some embodiments, the compositions for enabling adaptation, e.g., vectors, are cured from the cell via interference from the adapted non-adapting CRISPR-Cas system. For example, the adapted non-adapting CRISPR-Cas system may acquire spacers that target the compositions for enabling adaptation. In this case, the compositions for enabling adaptation would be cleaved and/or degraded by the adapted non-adapting CRISPR-Cas system. In some embodiments, the compositions for enabling adaptation, e.g., vectors, are cured by treating the bacteria with chemical or physical agents.

In some embodiments, for example when the composition for enabling adaptation is under control of an inducible promoter, the bacterial cells are removed from inducing conditions (see, e.g., Section I-B1).

In some embodiments, the curing, removing, or preventing expression of the compositions for enabling adaptation from the bacterial strains prevents the cells from continuing to acquire spacer sequences. The ability to control when adaptation occurs can have a number of advantages, including preventing additional spacer acquisition after the preferred immune profile is acquired.

Thus, the methods provided herein allow for a customized immune profile to be achieved in a (adapted) non-adapting CRISPR-Cas system, without the risk of the immune profile being modified by unwanted spacer acquisition.

### 3. Recipient Bacteria

It is further contemplated that the adapted non-adapting CRISPR-Cas system containing a customized immune profile may be introduced into recipient bacterial strains, e.g., recipient bacterial cells of a bacterial strain. In some embodiments, for example when the adapted non-adapting CRISPR-Cas system is contained on a plasmid, the plasmid may be introduced to a recipient bacterial strain. In some embodiments, a region of the adapted non-adapting CRISPR-Cas system containing one or more new spacer sequences may be introduced to a recipient bacterial strain. In some embodiments, the region of the adapted non-adapting CRISPR-Cas system containing the one or more new spacer sequences may be amplified, e.g., by PCR, and multiple copies may be introduced to the recipient bacterial strain. In some embodiments, the copies introduced to the recipient bacterial strain may undergo homologous recombination in the recipient cell. In some embodiments, the region of the adapted non-adapting CRISPR-Cas system containing the one or more new spacer sequences may be amplified, e.g., by PCR, and subcloned into an expression vector that can be introduced to a recipient bacterial strain.

In some embodiments, introduction to the recipient bacterial strain can be carried out by methods including, but not limited to, transformation, conjugation, transduction, or protoplast fusion. Methods for introducing polynucleotides or polypeptides by transformation into a recipient bacterial strain, include, but are not limited to, microinjection, electroporation, stable transformation methods, transient transformation methods (such as induced competence using chemical (e.g. divalent cations such as CaCl₂), mechanical (electroporation) means, or methods such as those described in published international applications WO 2018/114983 and WO 2010/149721, which are incorporated herein by reference in their entireties), ballistic particle acceleration (particle bombardment), direct gene transfer, viral-mediated introduction, cell-penetrating peptides, or mesoporous silica nanoparticle (MSN)-mediated direct protein delivery. Introducing a nucleic acid, construct, plasmid, or vector into a recipient bacterial strain can be carried out by conjugation, which is a specific method of natural DNA exchange requiring physical cell-to-cell contact. Introducing a nucleic acid, construct, plasmid, or vector into a recipient bacterial strain can be carried out by transduction, which is the introduction of DNA via a virus (e.g. phage) infection which is also a natural method of DNA exchange. Generally, such methods involve incorporating a polynucleotide within a viral DNA or RNA molecule.

Proteins may be introduced into a recipient bacterial strain by directly introducing the protein itself or an mRNA encoding the protein. The protein can be introduced into a recipient bacterial strain transiently. Uptake of the protein into the recipient bacterial strain can be facilitated with a Cell Penetrating Peptide (CPP).

In some embodiments, the introduction is stable, i.e., that the nucleic acid (construct, plasmid, or vector) introduced into the recipient bacterial strain integrates into a genome of the cell and is capable of being inherited by the progeny thereof. In some embodiments, the introduction can be temporary, i.e., that a nucleic acid (construct, plasmid, or vector) is introduced into the recipient bacterial strain and does not integrate into a genome of the cell or a polypeptide is introduced into the recipient bacterial strain. Transient transformation indicates that the introduced nucleic acid or protein is only temporarily expressed or present in the recipient bacterial strain. In some embodiments, transient introduction enables curing from the recipient bacterial strain.

In some embodiments, introduction to the recipient bacterial strain occurs by natural competence. In some embodiments, the recipient bacterial strain is naturally competent. In some embodiments, the natural competence is induced natural competence. For example, the recipient strain is induced to become competent. Methods of inducing natural competence in bacterial cells are known in the art, and in some cases may proceed according to the methods described in published international applications WO 2018/114983 and WO 2010/149721, which are incorporated herein by reference in their entireties. In some embodiments, introduction to the recipient bacterial strain occurs by conjugation. In some embodiments, introduction to the recipient bacterial strain occurs by transformation.

In some embodiments, the recipient bacterial strain is a strain of a starter culture. In some embodiments, the recipient bacterial strain is a strain of a protective culture. In some embodiments, the recipient bacterial strain is a strain of a probiotic culture. In some embodiments, the bacterial strain is a Gram-positive bacterial strain. In some embodiments, the bacterial strain is a lactic acid bacterial strain.

In some embodiments, the recipient bacterial strain is a Streptococcus thermophilus strain, a Lactobacillus acidophilus strain, a Bifidobacterium lactis strain, Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, a Lactococcus lactis strain, or a Lactococcus cremoris. In some embodiments, the recipient strain is a Lactococcus lactis or a biovar or subspecies thereof. In some embodiments, the recipient strain is a Lactococcus lactis subsp lactis. In some embodiments, the recipient strain is a Lactococcus cremoris or a biovar or subspecies thereof. In some embodiments, the recipient strain is a Lactococcus cremoris subsp cremoris. Examples of suitable bacterial strains to receive adapted non-adapting CRISPR-Cas systems are further described in Section I-D above.

In some embodiments, the adapted non-adapting CRISPR-Cas system is the only CRISPR-Cas system in the recipient bacterial strain. In some embodiments, the recipient bacterial strain includes one or more CRISPR-Cas systems in addition to the introduced adapted non-adapting CRISPR-Cas system or region containing the one or more new spacer sequences. In some embodiments, the region containing the one or more new spacer sequences is incorporated into a CRISPR-Cas system resident in the recipient bacterial strain. In some embodiments, the recipient bacterial strain is incapable of enabling adaptation in the adapted non-adapting CRISPR-Cas system, e.g., unless the composition and methods provided herein are performed.

### III. CULTURES AND PRODUCTS

Bacteria containing adapted non-adapting CRISPR-Cas systems, e.g., according to the methods provided herein, may be used in a variety of applications. In some embodiments, the bacteria may be used in methods of manufacturing food products. In some embodiments, the bacteria are or are a part of bacterial cultures for manufacturing food products.

### A. Bacterial Cultures and Methods of Use

In an aspect is provided a cell culture containing or consisting of at least one bacterial strain of the invention containing an adapted non-adapting CRISPR-Cas system as described herein. In some embodiments, the cell culture is a pure culture, i.e., comprises or consists of a single bacterial strain. In another embodiment, the cell culture is a mixed culture, i.e. comprises or consists of at least one bacterial strain(s) of the invention (containing an adapted non-adapting CRISPR-Cas system) and at least one other bacterial strain. By “at least one other bacterial strain”, it is meant 1 or more, and in particular 1, 2, 3, 4 or 5 strains. In some embodiments, the cell culture contains about 10 to about 40 bacterial strains, e.g., 10 to 35, 10 to 30, 10 to 25, 10 to 20, or 10 to 15 bacterial strains. In some embodiments, the cell culture contains at least 10 bacterial strains. In some embodiments, the cell culture contains at most 40 bacterial strains. In some embodiments, the cell culture contains at least 10 and at most 40 bacterial strains.

In some embodiments, the bacterial strain containing the adapted non-adapting CRISPR-Cas system is a lactic acid bacterial strain. In some embodiments, the bacterial strain containing the adapted non-adapting CRISPR-Cas system is from any of the following genera Streptococcus, Lactobacillus, Bifidobacterium, Limosilactobacillus, Lacticaseibacillus, Lactiplantibacillus, Propionibacteria, Pediococcus, Enterococcus, or Lactococcus. In some embodiments, the bacterial strain containing the adapted non-adapting CRISPR-Cas system is a Streptococcus thermophilus strain, a Lactobacillus acidophilus strain, a Bifidobacterium lactis strain, Limosilactobacillus fermentum strain, a Lacticaseibacillus paracasei strain, Lacticaseibacillus casei strain, Lacticaseibacillus rhamnosus strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, a Lactococcus lactis strain, or a Lactococcus cremoris. In some embodiments, the bacterial strain containing the adapted non-adapting CRISPR-Cas system is a Streptococcus thermophilus strain, a Lacticaseibacillus paracasei strain, a Lactiplantibacillus plantarum strain, a Lactobacillus delbrueckii subsp bulgaricus strain, a Propionibacteria freudenreichii strain, a Lactococcus lactis strain, or a Lactococcus cremoris. In some embodiments, the bacterial strain containing the adapted non-adapting CRISPR-Cas system is a Streptococcus thermophilus strain, a Lactococcus lactis strain, or a Lactococcus cremoris. In some embodiments, the bacterial strain containing the adapted non-adapting CRISPR-Cas system is a Lactococcus lactis strain. In some embodiments, the bacterial strain containing the adapted non-adapting CRISPR-Cas system is a Lactococcus lactis subsp lactis strain. In some embodiments, the bacterial strain containing the adapted non-adapting CRISPR-Cas system is a Lactococcus lactis biovar diacetylactis strain. In some embodiments, the bacterial strain containing the adapted non-adapting CRISPR-Cas system is a Lactococcus cremoris strain. In some embodiments, the bacterial strain containing the adapted non-adapting CRISPR-Cas system is a Lactococcus cremoris subsp cremoris strain.

In some embodiments, the cell culture of the invention includes or consists of at least one bacterial strain of the invention, and one or more further species of bacteria from the genera Streptococcus, Lactobacillus, Bifidobacterium, Limosilactobacillus, Lacticaseibacillus, Lactiplantibacillus, Propionibacteria, Pediococcus, Enterococcus, Lactococcus, or any combination thereof.

Lactococcus species include Lactococcus lactis, Lactococcus lactis subsp lactis, Lactococcus cremoris, Lactococcus cremoris subsp cremoris, and Lactococcus lactis biovar diacetylactis. Bifidobacterium species includes Bifidobacterium animalis, in particular Bifidobacterium animalis subsp lactis. Other lactic acid bacteria species include Leuconostoc sp., Streptococcus thermophilus, Lactobacillus acidophilus, Lactobacillus delbrueckii subsp. bulgaricus, Lactobacillus helveticus, Lacticaseibacillus rhamnosus, Lacticaseibacillus paracasei, and Lacticaseibacillus casei.

In some embodiments, the cell culture, either as a pure or mixed culture as defined above is in frozen, dried, freeze-dried, liquid or solid format, in the form of pellets or frozen pellets, or in a powder or dried powder. In some embodiments, the cell culture of the invention is in a frozen format or in the form of pellets or frozen pellets, in particular contained into one or more box or sachet. In some embodiments, the cell culture as defined herein is in a powder form, such as a dried or freeze-dried powder, in particular contained into one or more box or sachet.

In some embodiments, the cell culture of the invention, either as a pure culture or mixed culture as defined above, and whatever the format (frozen, dried, freeze-dried, liquid or solid format, in the form of pellets or frozen pellets, or in a powder or dried powder) comprises the bacterial strain(s) of the invention in a concentration comprised in the range of 10⁵ to 10¹² cfu (colony forming units) per gram of the cell culture. In some embodiments, the concentration of the bacterial strain(s) within the cell culture of the invention is in the range of 10⁷ to 10¹² cfu per gram of the cell culture, and in particular at least 10⁷, at least 10⁸, at least 10⁹, at least 10¹⁰ or at least 10¹¹ CFU/g of the cell culture. In a particular aspect, when in the form of frozen or dried concentrate, the concentration of bacterial strain(s) of the invention—as pure culture or as a mixed culture—within the cell culture is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

In some embodiments, the cell culture is a starter culture. In some embodiments, the starter culture is used to produce a food product. In some embodiments, the starter culture is used to produce a fermented food product. In some embodiments, the fermented food product is a fermented dairy product. In some embodiments, the fermented food product is a fermented dairy alternative product. For example, a plant-based fermented food product, such as a plant-based yoghurt, milk beverage, or cheese.

Starter cultures used in the manufacture of many fermented milk, cheese and butter products include cultures of bacteria, generally classified as lactic acid bacteria. Such bacterial starter cultures impart specific features to various dairy products by performing a number of functions.

Commercial non-concentrated cultures of bacteria are referred to in industry as ‘mother cultures’, and are propagated at the production site, for example a dairy, before being added to an edible starting material, such as milk, for fermentation.

The starter culture may comprise several bacterial strains, i.e. it may be a defined mixed culture. Accordingly, the starter culture may comprise the bacterial strain of the invention and a further bacterial strain, e.g., as described above.

For example, the starter culture may be suitable for use in the dairy industry. When used in the dairy industry the starter culture may additionally comprise a lactic acid bacteria species, a Bifidobacterium species, a Brevibacterium species, and/or a Propionibacterium species. Cultures of lactic acid bacteria are commonly used in the manufacture of fermented milk products—such as buttermilk, yoghurt or sour cream, and in the manufacture of butter and cheese, for example Brie or Harvati.

Suitable lactic acid bacteria include commonly used strains of a Lactococcus species, a Streptococcus species, a Lactobacillus species including Lactobacillus acidophilus, Enterococcus species, Pediococcus species, a Leuconostoc species and Oenococcus species or combinations thereof.

Other lactic acid bacteria species include Leuconostoc sp., Streptococcus thermophilus, Lactobacillus delbrueckii subsp. bulgaricus and Lactobacillus helveticus. Mesophilic cultures of lactic acid bacteria commonly used in the manufacture of fermented milk products such as buttermilk, yoghurt or sour cream, and in the manufacture of butter and cheese, for example Brie or Harvati. In addition, probiotic strains such as Bifidobacterium lactis, Lactobacillus acidophilus, Lacticaseibacillus casei may be added during said manufacturing to enhance flavour or to promote health.

Cultures of lactic acid bacteria commonly used in the manufacture of cheddar and Monterey Jack cheeses include Streptococcus thermophilus, Lactococcus lactis (e.g., Lactococcus lactis subsp lactis) and Lactococcus cremoris (e.g., Lactococcus cremoris subsp cremoris), or combinations thereof.

Thermophilic cultures of lactic acid bacteria commonly used in the manufacture of Italian cheeses such as Pasta filata or parmesan, include Streptococcus thermophilus and Lactobacillus delbrueckii subsp. bulgaricus. Other Lactobacillus species—such as Lactobacillus helveticus—may be added during manufacturing to obtain a desired flavour.

The selection of organisms for the starter culture of the invention will depend on the particular type of products to be prepared and treated. Thus, for example, for cheese and butter manufacturing, mesophilic cultures of Lactococcus species, Leuconostoc species and Lactobacillus, Lacticaseibacillus, Lactiplantibacillus, and Limosilactobacillus species are widely used, whereas for yoghurt and other fermented milk products, thermophilic strains of Streptococcus species and of Lactobacillus, Lacticaseibacillus, Lactiplantibacillus, and Limosilactobacillus species are typically used.

Starter cultures may be prepared by techniques well known in the art such as those disclosed in U.S. Pat. No. 4,621,058. By way of example, starter cultures may be prepared by the introduction of an inoculum, for example a bacterium, to a growth medium to produce an inoculated medium and ripening the inoculated medium to produce a starter culture.

Dried starter cultures may be prepared by techniques well known in the art, such as those discussed in U.S. Pat. Nos. 4,423,079 and 4,140,800.

In some embodiments, the cell culture is a protective culture. Protective cultures are considered in some cases as an integral part of starter cultures, which are the traditional tools of food technology used to produce fermented food such as cheese, yoghurt, certain sausages, wine etc. It is a general property of fermented foods that these possess a longer shelf life than the non-fermented raw materials (for instance cheese, has a much longer shelf-life than milk). This property is the result of the active metabolism of the fermenting culture, conducting its actions through a complex system of competition for nutrients and binding sites and by production of inhibitory metabolites like organic acids, hydrogen peroxide, diacetyl, reuterin and bacteriocins. Their usage is not limited to “classic” fermented foods but also plays an important role when their metabolic activities take place in food with a neutral pH and high water activity, which are subject to increased risk of growth of food pathogens. The application of protective cultures constitutes an additional measure to improve food hygiene and should not permit a neglecting of any measure of good manufacturing practice ensuring the high standard of food safety.

In some embodiments, the protective culture is used to protect food products. In some embodiments, the protective culture inhibits growth of biological contaminants, e.g., fungi, molds, harmful bacteria, in the food product. In some embodiments, the food product is a fermented food product. In some embodiments, the fermented food product is a fermented dairy product. In some embodiments, the fermented food product is a fermented dairy alternative product. For example, a plant-based fermented food product, such as a plant-based yoghurt alternative, plant-based milk beverage alternative, or plant-based cheese alternative. In some embodiments, the food product is a meat product. For example, a shelf-stable or cured meat product.

In some embodiments, the cell culture is a probiotic culture. In some embodiments, the probiotic culture is included in a food product. For example, the food product may provide nutritional benefit in addition to a therapeutic effect, such as in a nutritional supplement. Similarly, a food product may be formulated to enhance the taste of the probiotic culture or to make the probiotic culture more attractive to consume by being more similar to a common food item, rather than to a pharmaceutical composition. In some embodiments, the food product is a fruit juice; bar; cheese; fresh fermented product; pickle; kimchi; miso; kombucha; kefir or other fermented milks; tempeh; indigenous fermented food; sauerkraut and other fermented vegetables; coffee; cocoa and other fermented food containing yeast; sourdough bread; beer; cereal; milk; powder milk; infant formula; a composition for sportsmen like energy drinks, protein solutions/powders; specialized nutrition, e.g., for elderly or infants; hospital nutrition; medical foods. In some embodiments, the probiotic is included in a dietary supplement. In some embodiments, the probiotic culture is included in a functional food as defined herein.

### B. Products

Any product that is prepared from or contains a bacterial strain or cell culture of the invention is contemplated in accordance with the present invention. Suitable products include, but are not limited to, a food or a feed product. These include, but are not limited to, fruits, legumes, fodder crops and vegetables including derived products, grain and grain-derived products, dairy foods and dairy food-derived products, meat, poultry and seafood. In some embodiments, the food or feed product is a dairy, meat or cereal product.

The term “food” is used in a broad sense and includes feeds, foodstuffs, food ingredients, food supplements, and functional foods. Here, the term “food” is used in a broad sense—and covers food for humans as well as food for animals (i.e. a feed). In a preferred aspect, the food is for human consumption.

As used herein the term “food ingredient” includes a formulation, which is or can be added to foods and includes formulations which can be used at low levels in a wide variety of products that require, for example, acidifying or emulsifying.

As used herein, the term “functional food” means a food which is capable of providing not only a nutritional effect and/or a taste satisfaction but is also capable of delivering a further beneficial effect to consumer. Although there is no legal definition of a functional food, most of the parties with an interest in this area agree that there are foods marketed as having specific health effects.

The bacterial strain or cell culture of the present invention may be—or may be added to—a food ingredient, a food supplement, or a functional food. In some embodiments, the bacterial strain or cell culture of the present invention may be—or may be added to—a dietary supplement.

The food may be in the form of a solution or as a solid—depending on the use and/or the mode of application and/or the mode of administration.

The bacterial strain or cell culture of the present invention can be used in the preparation of food products such as one or more of: confectionery products, dairy products, meat products, poultry products, fish products and bakery products. By way of example, the bacterial strain or cell culture can be used as an ingredient to prepare soft drinks, a fruit juice or a beverage comprising whey protein, health teas, cocoa drinks, milk drinks and lactic acid bacteria drinks, yoghurt, drinking yoghurt and wine.

In some embodiments, a food as described herein is a dairy product. In some embodiments, a dairy product as described herein is one or more of the following: a yoghurt, a cheese (such as an acid curd cheese, a hard cheese, a semi-hard cheese, a cottage cheese), a buttermilk, quark, a sour cream, kefir, a fermented whey-based beverage, a koumiss, a milk beverage, a yoghurt drink, a fermented milk, a matured cream, a cheese, a fromage frais, a milk, a dairy product retentate, a process cheese, a cream dessert, or infant milk.

In some embodiments, a food as described herein is a fermented food product. In some embodiments, a food as described herein is a fermented dairy product—such as a fermented milk, a yoghurt, a cream, a matured cream, a cheese, a fromage frais, a milk beverage, a processed cheese, a cream dessert, a cottage cheese, a yoghurt drink, a dairy product retentate, or infant milk. In some embodiments, the dairy product according to the invention comprises milk of animal and/or plant origin.

Milk is understood to mean that of animal origin, such as cow, goat, sheep, buffalo, zebra, horse, donkey, or camel, and the like. The term milk also applies to a plant-based milk, for example extracts of plant material which have been treated or otherwise, such as leguminous plants (soya bean, chick pea, lentil and the like) or oilseeds (colza, soya bean, sesame, cotton and the like), which extract contains proteins in solution or in colloidal suspension, which are coagulable by chemical action, by acid fermentation and/or by heat. The word milk also denotes mixtures of animal milks and of vegetable milks.

The milk may be in the native state, reconstituted milk, a skimmed milk or a milk supplemented with compounds necessary for the growth of the bacteria or for the subsequent processing of fermented milk, such as fat, proteins of a yeast extract, peptone and/or a surfactant, for example.

In one embodiment, the term “milk” means commercial UHT milk supplemented with 3% (w/w) of semi-skimmed milk powder pasteurized by heating during 10 min+/−1 min. at 90° C.+/−0.2° C.

In a further aspect there is provided a method for preparing a food product, e.g., fermented food product, including fermenting a substrate with a bacterial strain or cell culture as described herein, e.g., by inoculating the substrate, to obtain a fermented product. In some embodiments, the bacterial strain of the invention is inoculated as a cell culture according to the present invention, such as a pure culture or a mixed culture. In some embodiments, the substrate is a milk substrate. By “milk substrate,” it is meant milk of animal and/or plant origin. In some embodiments, the milk substrate is of animal origin, such as cow, goat, sheep, buffalo, zebra, horse, donkey, or camel, and the like. The milk may be in the native state, a reconstituted milk, a skimmed milk, or a milk supplemented with compounds necessary for the growth of the bacteria or for the subsequent processing of fermented milk. In some embodiments, the milk substrate includes solid items. In some embodiments, the solid items include of fruits, chocolate products, or cereals. In some embodiments, the fermented product is a fermented dairy product.

In some embodiments, the food product is a meat, such as a sausage, a pepperoni, a salami, a ham, a frankfurter, a mortadella. In some embodiments, the meat is a fermented meat.

The present invention also provides in a further aspect the use of the bacterial strain or bacterial composition according to the present invention to manufacture a food or feed product, optionally a fermented food product, such as a fermented dairy product.

The invention is also directed to a food product. In some embodiments, the food product is obtained using the bacterial strain or cell culture of the invention or contains the bacterial strain or cell culture of the invention. In some embodiments, the food product is a yoghurt, a cream, a matured cream, a cheese, fromage frais, a milk beverage, a processed cheese, a cream dessert, a cottage cheese, an infant milk, a kefir, a sausage, a pepperoni, a salami, a ham, a frankfurter, a mortadella.

### IV. KITS

Also provided are kits including the compositions, e.g., non-adapting CRISPR-Cas systems, polynucleotides, vectors, bacteria, adapted non-adapting CRISPR-Cas systems, described herein, which may further include instructions on methods of using the compositions, such as uses described herein. In some embodiments, the kit includes polynucleotides encoding Cas1 and Cas2, either separately or together, as described herein. In some embodiments, the kit includes vectors encoding Cas1 and Cas2, either separately or together, as described herein. In some embodiments, the kit includes a non-adapting CRISPR-Cas system as described herein. In some embodiments, the kit includes the polynucleotides and/or vectors and a non-adapting CRISPR-Cas system, all of where are as described herein. In some embodiments, the kit includes an adapted non-adapting CRISPR-Cas system or a portion thereof containing one or more new spacer sequences. In some embodiments, the adapted non-adapting CRISPR-Cas system or the portion thereof containing one or more new spacer sequences are contained in a vector. In some embodiments, the portion of the adapted non-adapting CRISPR-Cas system containing one or more new spacer sequences are contained in a vector, optionally one or more vectors. The kits described herein may also include other materials desirable from a commercial and user standpoint, including other buffers, diluents, filters, needles, syringes, and package inserts with instructions for performing any methods described herein.

### Exemplary Embodiments

Among the provided embodiments are:

1. A polynucleotide comprising a nucleic acid sequence encoding a Clustered Regularly Interspaced Short Palindromic Repeats (CRISPR)-associated protein 1 (Cas1) polypeptide.
2. The polynucleotide of embodiment 1, wherein the nucleic acid sequence encoding the Cas1 polypeptide is operably linked to a heterologous regulatory sequence.
3. The polynucleotide of embodiment 1 or embodiment 2, comprising a nucleic acid sequence encoding a CRISPR-associated endoribonuclease Cas2 (Cas2) polypeptide. It is to be understood that such polynucleotide will comprise both a nucleic acid sequence encoding a Cas1 polypeptide and a nucleic acid sequence encoding a Cas2 polypeptide.
4. The polynucleotide of embodiment 3, wherein the heterologous regulatory sequence is a first heterologous regulatory sequence and the nucleic acid sequence encoding the Cas2 polypeptide is operably linked to a second heterologous regulatory sequence or a polycistronic element.
5. A polynucleotide comprising a nucleic acid sequence encoding a CRISPR-associated endoribonuclease Cas2 (Cas2) polypeptide.
6. The polynucleotide of embodiment 5, wherein the nucleic acid sequence encoding the Cas2 polypeptide is operably linked to a heterologous regulatory sequence.
7. The polynucleotide of embodiment 5 or embodiment 6, comprising a nucleic acid sequence encoding a Clustered Regularly Interspaced Short Palindromic Repeats (CRISPR)-associated protein 1 (Cas1) polypeptide.
8. The polynucleotide of embodiment 7, wherein the heterologous regulatory sequence is a first heterologous regulatory sequence and the nucleic acid sequence encoding the Cas1 polypeptide is operably linked to a second heterologous regulatory sequence or a polycistronic element.
9. The polynucleotide of any one of embodiments 1-4, 7, and 8, wherein the Cas1 polypeptide is a Cas1 polypeptide of a Lactococcus bacterial strain.
10. The polynucleotide of any one of embodiments 1-4, and 7-9, wherein the Cas1 polypeptide is a Cas1 polypeptide of a Lactococcus raffinolactis bacterial strain.
11. The polynucleotide of any one of embodiments 1-4, and 7-9, wherein the Cas1 polypeptide is a Cas1 polypeptide of a Lactococcus lactis bacterial strain, a Lactococcus cremoris bacterial strain, or a subspecies or biovar thereof.
12. The polynucleotide of any one of embodiments 1-4 and 7-10, and wherein the Cas1 polypeptide comprises an amino acid sequence set forth in SEQ ID NO:2 or an amino acid sequence having at least 85%, 90%, 91%, 92%, 93%, 94%, 95%, 96%, 97%, 98%, or 99% sequence identity to the sequence set forth in SEQ ID NO:2.
13. The polynucleotide of any one of embodiments 3-12, wherein the Cas2 polypeptide is a Cas2 polypeptide of a Lactococcus bacterial strain.
14. The polynucleotide of any one of embodiments 3-13, wherein the Cas2 polypeptide is a Cas2 polypeptide of a Lactococcus raffinolactis bacterial strain.
15. The polynucleotide of any one of embodiments 3-13, wherein the Cas2 polypeptide is a Cas2 polypeptide of a Lactococcus lactis bacterial strain, a Lactococcus cremoris bacterial strain, or a subspecies or biovar thereof.
16. The polynucleotide of any one of embodiments 3-14, wherein the Cas2 polypeptide comprises an amino acid sequence set forth in SEQ ID NO:4 or an amino acid sequence having at least 85%, 90%, 91%, 92%, 93%, 94%, 95%, 96%, 977%, 98%, or 99% sequence identity to the sequence set forth in SEQ ID NO:4.
17. The polynucleotide of any one of embodiments 3, 4, and 8-16, wherein the nucleic acid sequence encoding the Cas1 polypeptide is 5′ to the nucleic acid sequence encoding the Cas2 polypeptide.
18. The polynucleotide of any one of embodiments 4 and 7-16, wherein the nucleic acid sequence encoding the Cas2 polypeptide is 5′ to the nucleic acid sequence encoding the Cas1 polypeptide.
19. The polynucleotide of any one of embodiments 2, 3, 4, 6-18, wherein the heterologous regulatory sequence is a promoter.
20. The polynucleotide of any one of embodiments 4 and 8-19, wherein the first and/or second heterologous regulatory sequence is a promoter.
21. The polynucleotide of embodiment 19 or embodiment 20, wherein the promoter is an inducible promoter.
22. The polynucleotide of embodiment 21, wherein the inducible promoter is a phage-inducible promoter or a chemical-inducible promoter.
23. The polynucleotide of any one of embodiments 4 and 8-22, wherein the polycistronic element is a ribosome binding site.
24. A vector comprising the polynucleotide of any one of embodiments 1, 2, 9-12, 19, 21, and 22.
25. A vector comprising the polynucleotide of any one of embodiments 5, 6, 13-16, 19, 21, and 22.
26. A vector comprising the polynucleotide of any one of embodiments 3, 4, and 7-23.
27. The vector of any one of embodiments 24-26, wherein the vector is labile.
28. A bacterial strain comprising one of:
(i) a vector of embodiment 26 or embodiment 27;
(ii) a first vector and a second vector, wherein the first vector is a vector of embodiment 24 or embodiment 27 and the second vector is a vector of embodiment 25 or embodiment 27;
(iii) a first polynucleotide and a second polynucleotide, wherein the first polynucleotide is a polynucleotide of any one of embodiments 1, 2, 9-12, 19, 21, and 22 and the second polynucleotide is a polynucleotide of any one of embodiments 5, 6, 13-16, 19, 21, and 22; or
(iv) a polynucleotide of any one of embodiments 3, 4, and 7-23.
   29. The bacterial strain of embodiment 28, wherein the bacterial strain comprises a non-adapting CRISPR-Cas system.
   30. The bacterial strain of embodiment 29, wherein the non-adapting CRISPR-Cas system is comprised in a plasmid.
   31. The bacterial strain of embodiment 29 or embodiment 30, wherein the non-adapting CRISPR-Cas system is a lactococcal CRISPR-Cas system, optionally a Lactococcus cremoris, a Lactococcus lactis, or a subspecies or biovar thereof CRISPR-Cas system.
   32. The bacterial strain of any one of embodiments 29-31, wherein the non-adapting CRISPR-Cas system is a type III-A CRISPR-Cas system.
   33. The bacterial strain of any one of embodiments 29-32, wherein the non-adapting CRISPR-Cas system comprises a nucleic acid sequence encoding the set forth by SEQ ID NO:24 or a sequence having at least 85%, 90%, 91%, 92%, 93%, 94%, 95%, 96%, 97%, 98%, or 99% sequence identity to the sequence set forth in SEQ ID NO:24.
   34. A method of enabling adaptation in a non-adapting CRISPR-Cas system, the method comprising introducing into a bacterial strain comprising a non-adapting CRISPR-Cas system one of:
(i) a vector of any embodiment 26 or 27;
(ii) a first vector and a second vector, wherein the first vector is a vector of embodiment 24 or embodiment 27 and the second vector is a vector of embodiment 25 or embodiment 27;
(iii) a first polynucleotide and a second polynucleotide, wherein the first polynucleotide is a polynucleotide of any one of embodiments 1, 2, 9-12, 19, 21, or 22 and the second polynucleotide is a polynucleotide of any one of embodiments 5, 6, 13-16, 19, 21, or 22; or
(iv) a polynucleotide of any one of embodiments 3, 4, and 7-23.
   35. The method of embodiment 34, further comprising exposing the bacterial strain to a target nucleic acid.
   36. The method of embodiment 34 or embodiment 35, further comprising exposing the bacterial strain to a bacteriophage.
   37. The method of any one of embodiments 34-36, further comprising exposing the bacterial strain to one or more stressors or selective pressures dependent on a desirable phenotype.
   38. A method of producing a bacterial strain resistant to a nucleic acid, the method comprising exposing a bacterial strain to a target nucleic acid, wherein the bacterial strain comprises a non-adapting CRISPR-Cas system and one of:
(i) a vector of embodiment 26 or embodiment 27;
(ii) a first vector and a second vector, wherein the first vector is a vector of embodiment 24 or embodiment 27 and the second vector is a vector of embodiment 25 or embodiment 27;
(iii) a first polynucleotide and a second polynucleotide, wherein the first polynucleotide is a polynucleotide of any one of embodiments 1, 2, 9-12, 19, 21, and 22 and the second polynucleotide is a polynucleotide of any one of embodiments 5, 6, 13-16, 19, 21, and 22; or
(iv) a polynucleotide of any one of embodiments 3, 4, and 7-23.
   39. The method of any one of embodiments 35-38, wherein the target nucleic acid is a foreign DNA or foreign RNA.
   40. The method of any of embodiments 35-39, wherein the target nucleic acid is a plasmid or mobile genetic element.
   41. The method of embodiment 39 or embodiment 40, wherein the foreign DNA or foreign RNA encodes an antibiotic resistance, a virulence factor, or a toxin.
   42. The method of any one of embodiments 35-41, wherein the exposing comprises contacting the bacterial strain with one or more target nucleic acids, optionally simultaneously or sequentially.
   43. The method of any one of embodiments 35-42, comprising selecting and/or isolating bacterial cells resistant to the target nucleic acid.
   44. A method of producing a bacterial strain resistant to a bacteriophage, the method comprising exposing a bacterial strain to a bacteriophage, wherein the bacterial strain comprises a non-adapting CRISPR-Cas system and one of:
(i) a vector of embodiment 26 or embodiment 27;
(ii) a first vector and a second vector, wherein the first vector is a vector of embodiment 24 or embodiment 27 and the second vector is a vector of embodiment 25 or embodiment 27;
(iii) a first polynucleotide and a second polynucleotide, wherein the first polynucleotide is a polynucleotide of any one of embodiments 1, 2, 9-12, 19, 21, and 22 and the second polynucleotide is a polynucleotide of any one of embodiments 5, 6, 13-16, 19, 21, and 22; or
(iv) a polynucleotide of any one of embodiments 3, 4, and 7-23.
   45. The method of any one of embodiments 36-37 and 39-44, wherein the exposing comprises contacting the bacterial strain with one or more bacteriophages, optionally simultaneously or sequentially.
   46. The method of any one of embodiments 36-37 and 39-45, comprising selecting and/or isolating bacterial cells resistant to the bacteriophage.
   47. A method of producing a bacterial strain having a desirable phenotype, the method comprising exposing a bacterial strain to one or more stressors or selective pressures dependent on a desirable phenotype, wherein the bacterial strain comprises a non-adapting CRISPR-Cas system and one of:
(i) a vector of embodiment 26 or embodiment 27;
(ii) a first vector and a second vector, wherein the first vector is a vector of embodiment 24 or embodiment 27 and the second vector is a vector of embodiment 25 or embodiment 27;
(iii) a first polynucleotide and a second polynucleotide, wherein the first polynucleotide is a polynucleotide of any one of embodiments 1, 2, 9-12, 19, 21, and 22 and the second polynucleotide is a polynucleotide of any one of embodiments 5, 6, 13-16, 19, 21, and 22; or
(iv) a polynucleotide of any one of embodiments 3, 4, and 7-23.
   48. The method of any one of embodiments 37, 39-43, and 45-47, wherein the desirable phenotype is a phenotype useful for food production, food protection, or a probiotic.
   49. The method of any one of embodiments 37, 39-43, and 45-48, wherein the one or more stressors or selective pressures comprise starvation, nutrient-limitation, nutritional selection, hunger, temperature selection, oxidative stress, toxins, antibiotics, low pH, alkylation stress, or osmotic pressure.
   50. The method of any one of embodiments 37, 39-43, and 45-49, comprising selecting and/or isolating bacterial cells of the bacterial strain having the desirable phenotype.
   51. The method of any one of embodiments 34-50, wherein the one of:
(i) the vector of embodiment 26 or embodiment 27;
(ii) the first vector and a second vector, wherein the first vector is a vector of embodiment 24 or embodiment 27 and the second vector is a vector of embodiment 25 or embodiment 27;
(iii) a first polynucleotide and a second polynucleotide, wherein the first polynucleotide is a polynucleotide of any one of embodiments 1, 2, 9-12, 19, 21, and 22 and the second polynucleotide is a polynucleotide of any one of embodiments 5, 6, 13-16, 19, 21, and 22; or (iv) a polynucleotide of any one of embodiments 3, 4, and 7-23;
is introduced to the bacterial strain prior to exposing.
   52. The method of any one of embodiments 34-51, wherein the non-adapting CRISPR-Cas system is comprised on a plasmid.
   53. The method of any one of embodiments 34-52, wherein the non-adapting CRISPR-Cas system is a type III-A CRISPR-Cas system.
   54. The method of any one of embodiments 34-53, wherein the non-adapting CRISPR-Cas system is a lactococcal CRISPR-Cas system, optionally a Lactococcus cremoris or a Lactococcus lactis CRISPR-Cas system.
   55. The method of any one of embodiments 34-54, wherein the non-adapting CRISPR-Cas system comprises a nucleic acid sequence encoding the sequence set forth by SEQ ID NO:24 or a sequence having at least 95%, 96%, 97%, 98%, or 99% sequence identity to the sequence set forth in SEQ ID NO:24.
   56. The method of any one of embodiments 43, 45, 46, and 48-50, wherein the non-adapting CRISPR-Cas system of the selected and/or isolated bacterial cells comprises one or more new spacer sequences compared to spacers of the non-adapting CRISPR-Cas system prior to the exposing.
   57. The method of embodiment 56, wherein the one or more new spacer sequences are identified, and optionally sequenced.
   58. The method of any one of embodiments 43, 45, 46, and 48-57, further comprising removing the one of:
(i) a vector of embodiment 26 or embodiment 27;
(ii) a first vector and a second vector, wherein the first vector is a vector of embodiment 24 or embodiment 27 and the second vector is a vector of embodiment 25 or embodiment 27; or
(iii) a first polynucleotide and a second polynucleotide, wherein the first polynucleotide is a polynucleotide of any one of embodiments 1, 2, 9-12, 19, 21, and 22 and the second polynucleotide is a polynucleotide of any one of embodiments 5, 6, 13-16, 19, 21, and 22; or
(iv) a polynucleotide of any one of embodiments 3, 4, and 7-23;
from the selected and/or isolated bacterial cells.
   59. The method of any one of embodiments 43, 45, 46, and 48-58, further comprising introducing the non-adapting CRISPR-Cas system of the selected and/or isolated bacterial cells that comprises one or more new spacer sequences to a recipient bacterial strain.
   60. The method of embodiment 59, wherein the introducing comprises:
(i) introducing the non-adapting CRISPR-Cas system comprising the one or more new spacer sequences to the recipient bacterial strain, optionally wherein the non-adapting CRISPR-Cas system is comprised in a plasmid; or
(ii) introducing a region of the non-adapting CRISPR-Cas system comprising the one or more new spacer sequences to the recipient bacterial strain, optionally wherein the region comprising the one or more spacer sequences is amplified and multiple copies of the region are introduced to the recipient bacterial strain, optionally in a vector.
   61. The method of embodiment 59 or embodiment 60, wherein the introducing comprises conjugation, transformation, and/or transduction, and optionally wherein the recipient bacterial strain is naturally competent.
   62. The method of any one of embodiments 59-61, wherein the recipient bacterial strain is a strain of a starter culture, a protective culture, or a probiotic culture.
   63. The method of any one of embodiments 59-62, wherein the recipient bacterial strain is a lactic acid bacterial strain.
   64. The method of any one of embodiments 59-63, wherein the recipient bacterial strain is a Streptococcus thermophilus strain, a Lactobacillus acidophilus strain, a Bifidobacterium lactis strain, Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, a Lactococcus lactis strain, or a Lactococcus cremoris strain.
   65. The method of any one of embodiments 59-64, wherein the recipient bacterial strain is a Lactococcus lactis strain, a Lactococcus cremoris strain, or a biovar or subspecies thereof.
   66. The method of any one of embodiments 34-65, wherein bacterial strain is a strain of a starter culture, a protective culture, or a probiotic culture.
   67. The method of any one of embodiments 34-66, wherein bacterial strain is a Lactococcus lactis strain, a Lactococcus cremoris strain, or a biovar or subspecies thereof.
   68. A bacterial strain produced by the method of any one of embodiments 34-67.
   69. The bacterial strain of any one of embodiments 28-33 and 68, wherein the bacterial strain is a strain of a starter culture, a probiotic culture, or a protective culture.
   70. The bacterial strain of any one of embodiments 28-33, 68, and 69, wherein the bacterial strain is a Lactococcus lactis, Lactococcus cremoris, or a biovar or subspecies thereof.
   71. An adapted non-adapting CRISPR-Cas system produced by the method of any one of embodiments 34-67, optionally comprised on a plasmid.
   72. A bacterial strain comprising the adapted non-adapting CRISPR-Cas system of embodiment 71 or a portion of the adapted non-adapting CRISPR-Cas system comprising one or more new spacer sequences.
   73. The bacterial strain of embodiment 72, wherein the bacterial strain is a strain of a starter culture, a probiotic culture, or a protective culture.
   74. The bacterial strain of embodiment 72 or embodiment 73, wherein the bacterial strain is a lactic acid bacterial strain.
   75. The bacterial strain of any one of embodiments 72-74, wherein the bacterial strain is a Lactococcus bacterial strain.
   76. The bacterial strain of any one of embodiments 72-75, wherein the bacterial strain is a Streptococcus thermophilus strain, a Lactobacillus acidophilus strain, a Bifidobacterium lactis strain, Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, a Lactococcus lactis strain, or a Lactococcus cremoris strain.
   77. The bacterial strain of any one of embodiments 72-76, wherein the bacterial strain is a Lactococcus lactis strain, a Lactococcus cremoris strain, or a biovar or subspecies thereof.
   78. A cell culture comprising the bacterial strain of any one of embodiments 68-70 or the bacterial strain of any one of embodiments 72-77.
   79. The cell culture according to embodiment 78, wherein said culture is a starter culture, a probiotic culture, or a protective culture.
   80. The cell culture of embodiment 79, further comprising one or more strains selected from the group consisting of a Streptococcus thermophilus strain, a Lactococcus cremoris strain, a Lactobacillus acidophilus strain, a Bifidobacterium lactis strain, Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, and a Lactococcus lactis strain.
   81. A food product comprising the bacterial strain of any one of embodiments 72-77 or the cell culture of any one of embodiments 78-80.
   82. A dietary supplement comprising the bacterial strain of any one of embodiments 72-77 or the cell culture of any one of embodiments 78-80.
   83. Use of a bacterial strain of any one of embodiments 72-77 or the cell culture of any one of embodiments 78-80 for preparing a food product or dietary supplement.
   84. The food product of embodiment 81 or the use of embodiment 83, wherein the food product is a fermented food product.
   85. A method for preparing a food product, the method comprising fermenting a substrate with a bacterial strain of any one of embodiments 72-77 or a cell culture of any one of embodiments 78-80.
   86. The method of embodiment 85, wherein the substrate is a dairy milk or vegetal substrate.
   87. The food product of embodiment 81 or embodiment 83, the use of embodiment 83 or embodiment 84, or the method of embodiment 83 or embodiment 84, wherein the food product is a yoghurt, a cream, a matured cream, a cheese, fromage frais, a milk beverage, a processed cheese, a cream dessert, a cottage cheese, an infant milk, a kefir, a sausage, a pepperoni, a salami, a ham, a frankfurter, a mortadella, or a plant-based alternative of any of the foregoing.

### V. EXAMPLES

The following examples are included for illustrative purposes only and are not intended to limit the scope of the invention.

### Example 1: Induction of CRISPR Adaptation in Lactococcus

Clustered Regularly Interspaced Short Palindromic Repeats-CRISPR associated proteins (CRISPR-Cas) is a phage resistance mechanism found in bacteria and archaea. This adaptive immune system consists of short repeat sequences interspaced by short variable/spacer sequences derived from invading nucleic acids. CRISPR-Cas systems generally function in three steps: 1) adaptation, where new spacers are incorporated into the CRISPR array; 2) maturation or processing, during which CRISPR RNAs (crRNA) are generated to guide the Cas protein machinery towards their respective nucleic acid targets; 3) interference, in which the crRNA targeted invader nucleic acid is cleaved and/or degraded (Makarova et al., 2015, Nature Reviews, Microbiology, Vol. 13, November 2015, p. 722-736). The native CRISPR-Cas identified in milk-adapted lactococci, however, appears to lack the ability to adapt and incorporate new spacers into the CRISPR array (Millen et al., 2012, PLOS ONE, December 2012, Vol. 7, issue 12, e51663). The ability to enable CRISPR adaptation in Lactococcus cremoris subsp cremoris (L. cremoris subsp cremoris) was assessed.

### Materials and Methods

Exemplary strains, bacteriophages, and plasmids used for assessing the induction of CRISPR adaptation in L. cremoris subsp cremoris are shown in Table E1.

TABLE E1
Exemplary strains, bacteriophages, and plasmids for used for testing
induction of CRISPR adaptation in L. cremoris subsp cremoris.

| | |
|---|---|
| Biological Material | Relevant Characteristics |

Bacteria

| | |
|---|---|
| Lactococcus cremoris | Natural CRISPR-Cas transconjugant of MG1363 containing p537CR (Wegmann, |
| subsp cremoris | U., M. O'Connell-Motherway, A. Zomer, G. Buist, C. Shearman, C. Canchaya, |
| DGCC12607 | M. Ventura, A. Goesmann, M. J. Gasson, O. P. Kuipers, D. van Sinderen, and J. |
| | Kok. 2007. Complete genome sequence of the prototype lactic acid bacterium |
| | Lactococcus lactis subsp. cremoris MG1363. J. Bacteriol. 189: 3256-3270.) |
| Lactococcus | CRISPR-Cas⁺, cas1-cas2⁺ |
| raffinolactis Lr_19_5 | |
| (Accession number | |
| CP047616) | |
| Enterococcus italicus | CRISPR-Cas⁺, cas1-cas2⁺ |
| DSM15952 | |
| (Accession number | |
| AEPV01000074) | |
| 12607-989 | DGCC12607 + pTRK989 |
| 12607-Cas1Cas2 | DGCC12607 + pRafCas1Cas2 |
| 12607-Cas1 | DGCC12607 + pRafCas1 |
| 12607-Cas2 | DGCC12607 + pRafCas2 |
| 12607-italCas1Cas2 | DGCC12607 + pItalCas1Cas2 |
| Eschericia coli | Host for vector assembly |
| TG1RepA | |
| (Duwat et al., 1997) | |

Bacteriophages

| | |
|---|---|
| p2.S3.S4.V | Host DGCC12607; variant of p2 (Bebeacua et al., 2013, Structure, adsorption to |
| | host, and infection mechanism of virulent lactococcal phage p2. J. Virol. 87 |
| | 12302-12312); Accession number NC_042024) with mutations in protospacers |
| | 3 and 4 abrogating CRISPR-Cas directed phage resistance |

Plasmids

| | |
|---|---|
| p537CR | Native lactococcal CRISPR-Cas plasmid; CRISPR-Cas operon is identical to |
| | that on pKLM (Millen et al., 2012, PLOS ONE, December 2012, Vol. 7, |
| | issue 12, e51663) |
| pTRK989 | p6 promoter, Cm^{R} |
| (Rodrigues da | |
| Cunha, 2011) | |
| pRafCas1Cas2 | pTRK989 + L. raffinolactis cas1-cas2 |
| pRafCas1 | pTRK989 + L. raffinolactis cas1 |
| pRafCas2 | pTRK989 + L. raffinolactis cas2 |
| pItalCas1Cas2 | pTRK989 + E. italicus cas1-cas2 |

### Cas1-Cas2 Complementation

pRafCas1Cas2 and pltalCas1Cas2 were assembled in E. coli TG1RepA using NEBuilder HiFi DNA assembly. cas1-cas2 from L. raffinolactis Lr_19_5 was synthesized as a gBlock by Integrated DNA Technologies (USA) (Table E2). cas1-cas2 from E. italicus was amplified from DSM15952 (see, Table E2) with primers italCas1Cas2F and italCas1Cas2R. pTRK989 was amplified with primers 989RafCasF-GC and 989RafCasR-GC for assembly with the L. raffinolactis Cas1Cas2 gBlock and with primers 989F and 989F for assembly with E. italicus cas1-cas2 amplicon. PCRs were performed using Phusion® High-Fidelity PCR Master Mix with HF Buffer (New England Biolabs Inc., USA) according to manufacturer's instructions. Primers were synthesized by Integrated DNA Technologies (USA) (Table E2). PCR products were purified using Wizard SV Gel and PCR Clean-Up System (Promega Corp., Madison, WI, USA). Purified vector amplicons were assembled with the respective insert (L. raffinolactis Cas1Cas2 gBlock or E. italicus cas1-cas2 amplicon) using NEBuilder HiFi DNA Assembly MasterMix (New England Biolabs, USA) according to manufacturer's instructions. Assemblies were electroporated into TG1 RepA (Dower et al., 1988, Nucleic Acids Res. 1988 Jul. 11; 16(13): 6127-6145). Recombinant vectors were purified from E. coli using the GeneJET Plasmid Miniprep Kit (Thermo Fisher Scientific, USA). pRafCas1 and pRafCas2 were developed by amplifying regions of pRafCas1Cas2 and assembling the resultant amplicons using NEBuilder HiFi DNA assembly.

Amplicons generated from PCRs with primer set CasSingleF and Cas1cloneR and primer set CasSingleR and Cas1cloneF were assembled to develop pRafCas1, and amplicons generated from PCRs with primer set CasSingleF and Cas2clone-rbsR and primer set CasSingleR and Cas2clone-rbsF were assembled to develop pRafCas2. Purified vectors were transformed (Holo and Nes, 1989) into DGCC12607.

### Phage Challenge and Detection of Spacer Acquisition

A single-step phage challenge was performed by exposing DGCC12607 transformants to exemplary phage p2.S3.S4.V at two multiplicities of infection (MOI) which were then plated in MRS overlay. Plates were incubated at 30° C. for 2 days. Colonies that grew in the presence of p2.S3.S4.V were presumed to be phage-resistant variants. Colonies were tested for spacer acquisition using primers CR-F2 and Sp15R.

Spacer acquisition was tested in a culture population by PCR with primers CR-F2 (upstream of the first repeat) and Sp15R (inside of the first spacer) (see, Table E2) using overnight culture as a template. A faint higher molecular weight band in addition to the control amplicon was interpreted as positive for acquisition in the population.

TABLE E2
Primer sequences and cas1 and cas2 sequences.

| SEQ ID | | |
|---|---|---|
| NO: | Sequence (5′-3′) | Description |
| 1 | ATGAGAGACATTATCTACGTTGAAAATAGGTATTTTATCAGTAGTC | L. raffinolactis |
| | GAGAAAATGCGCTCAGATTTCATGACTATATTAATAAAGCGACCCA | Cas1Cas2 gBlock |
| | TTTCATTGCTTTTGATGACATTGATATTTTAGTTTTGGATAATGCAA | |
| | GAAGTTACTTGTCAAATGGTGTGATTAACGACTGTTTGGATCGAAA | |
| | TATTTTGATTTTAACTTGCGATAATAAACATTCTCCTAAAGCGATTT | |
| | TAAGCAATGCTTTTGCTAATAAAAAACGCTTAGAACGCTTAAGAAA | |
| | TCAATTGCAATTATCCTCTAAGTCAAAGAATCGTCTTTGGCGTAAA | |
| | ATTGTGATGGCGAAAATTAATAATCAGGCTGATGCTGTTACCTTCA | |
| | CGGTTCAAGATGGAACAGTTCATCGAGAGATAATTGAACTAGGAA | |
| | AAATGGTCACAGAAGGAGATAAAGATAATCGCGAAGCTGTCGTTG | |
| | CACGAAAATATTTTCGGACATTATTTGGTGGTAATTTTAAGCGTGG | |
| | TCGTTTTGATGATGTCATCAATTCAGCGCTCAATTATGGCTATGCA | |
| | CTTGTGAGGGCTGTCATCAGACGAGAATTGGCTATCTGTGGTTTT | |
| | GAGATGAGCTTTGGTATTCATCATATGTCAACAGAAAATCCTTTTA | |
| | ATCTCTCTGATGATATGATTGAAGTTTTTCGGCCTTTTGTTGATGT | |
| | GTTGGTATTTGAGATAATTGTGACGAATGACGTAACTGTTTTTGAT | |
| | TATGAAATCAAAAAACTTCTGGTTAATATTTTTCTGGAAAAATGTGT | |
| | GATAGACGGAAAAGTCATGTCTTTAACAGATGCAGTGCGTGTAAC | |
| | TATCCAGAGTTTAATTACTTGTCTTGAGGATGACAGCAGTGCACC | |
| | CTTGAAACTTCCTAGCTTTATTCAGGAGGGAAAATAGATGATGTTG | |
| | TTGTGTGCTTTTGATTTGCCTCGTGAGACCAAAGAAGAAAGAAAA | |
| | GCTGCAAATAAATACCGTAAGCGATTAGTAGAGCTTGGATTTGCA | |
| | ATGAAGCAATTTTCCTTGTATGAGCGTGAGGTTCGTCATATTGATG | |
| | TTAAAAATAGGTTAATTGATATTTTACGGGAAGAATTACCAGATAC | |
| | TGGAGCGATTACCCTATACCTATTACCCAATGAGGTGAATGATGC | |
| | TCAGATTACGATATTGGGTGAAAAGTCGGTTAATAAAACGGTAAG | |
| | AGTTGCAAGAATTATTTTTTTGTAG | |
| 3 | ATGAGAGACATTATCTACGTTGAAAATAGGTATTTTATCAGTAGTC | L. raffinolactis |
| | GAGAAAATGCGCTCAGATTTCATGACTATATTAATAAAGCGACCCA | cas1 |
| | TTTCATTGCTTTTGATGACATTGATATTTTAGTTTTGGATAATGCAA | |
| | GAAGTTACTTGTCAAATGGTGTGATTAACGACTGTTTGGATCGAAA | |
| | TATTTTGATTTTAACTTGCGATAATAAACATTCTCCTAAAGCGATTT | |
| | TAAGCAATGCTTTTGCTAATAAAAAACGCTTAGAACGCTTAAGAAA | |
| | TCAATTGCAATTATCCTCTAAGTCAAAGAATCGTCTTTGGCGTAAA | |
| | ATTGTGATGGCGAAAATTAATAATCAGGCTGATGCTGTTACCTTCA | |
| | CGGTTCAAGATGGAACAGTTCATCGAGAGATAATTGAACTAGGAA | |
| | AAATGGTCACAGAAGGAGATAAAGATAATCGCGAAGCTGTCGTTG | |
| | CACGAAAATATTTTCGGACATTATTTGGTGGTAATTTTAAGCGTGG | |
| | TCGTTTTGATGATGTCATCAATTCAGCGCTCAATTATGGCTATGCA | |
| | CTTGTGAGGGCTGTCATCAGACGAGAATTGGCTATCTGTGGTTTT | |
| | GAGATGAGCTTTGGTATTCATCATATGTCAACAGAAAATCCTTTTA | |
| | ATCTCTCTGATGATATGATTGAAGTTTTTCGGCCTTTTGTTGATGT | |
| | GTTGGTATTTGAGATAATTGTGACGAATGACGTAACTGTTTTTGAT | |
| | TATGAAATCAAAAAACTTCTGGTTAATATTTTTCTGGAAAAATGTGT | |
| | GATAGACGGAAAAGTCATGTCTTTAACAGATGCAGTGCGTGTAAC | |
| | TATCCAGAGTTTAATTACTTGTCTTGAGGATGACAGCAGTGCACC | |
| | CTTGAAACTTCCTAGCTTTATTCAGGAGGGAAAATAG | |
| 5 | ATGATGTTGTTGTGTGCTTTTGATTTGCCTCGTGAGACCAAAGAAG | L. raffinolactis |
| | AAAGAAAAGCTGCAAATAAATACCGTAAGCGATTAGTAGAGCTTG | cas2 |
| | GATTTGCAATGAAGCAATTTTCCTTGTATGAGCGTGAGGTTCGTCA | |
| | TATTGATGTTAAAAATAGGTTAATTGATATTTTACGGGAAGAATTAC | |
| | CAGATACTGGAGCGATTACCCTATACCTATTACCCAATGAGGTGA | |
| | ATGATGCTCAGATTACGATATTGGGTGAAAAGTCGGTTAATAAAAC | |
| | GGTAAGAGTTGCAAGAATTATTTTTTTGTAG | |
| 7 | ATGAAGGATATTATTTATGTTGAACGAAAGTATTTTTTAACCGTAAA | E. italicus cas1 |
| | AGGGGAGTCCATAAAATTCGTAAACGTGATGGACAAAACGGAAAA | |
| | ATACATCCCGCTTGATGATGTGGAATGGTTGATTTTTGATCATCCT | |
| | AGTAGTTATTTTTCAAACAAATTAATTACTGAATGTATGGTTAGAGG | |
| | GATAGGTGTGCTTTTTTGCGATGGCAAGCATTCACCAGATGCTAT | |
| | TTTACTGAATCAGTTTGGACATCGACAAAGATTAACGCGAGTGAAT | |
| | CATCAATTATCTGTATCGAATCGAACAAAAAAACGATTATGGAAGA | |
| | AAATAATCAGGACCAAAATTCATAATCAAGCCCAATGCATTGATCA | |
| | ATTAACAGATAACCAGAAGAGCAGAGATTACCTTATGTCTCTGTCA | |
| | AATTCTGTGCAGGAAGGAGACAGTTCAAATAGAGAGGCGGTGGC | |
| | AGCTAAGATTTACTTCACTGCATTATTTGGTGAAAACTTTAGGCGC | |
| | GGACGCTATGATGATGTTGTAAATAGTGGCTTAAATTATGGGTATG | |
| | CACTTGTTAGAGGAATGATCAAAAAAGAACTGGCAAGGTATGGTG | |
| | TAGAACAGAGCTTTGGCATCCATCATAAATCAAGTGAAAATCCGTT | |
| | TAATTTAGCTGATGACATTATTGAACCTTTTCGACCATTCGTAGAT | |
| | CGAAGTGTGTATGAAAATTTTTATTTGACAGAAAAAACGACTTTCG | |
| | AACATACTGACAAACAAGCACTATTTCAAGTTTTTTTTGATTCATGC | |
| | ATCATTCAAGGAAAAGTGTTTCAACTGACGGATGCCATTAATCAAG | |
| | CAGTGCAATCTTTCGTAAAATCAATTGATCAGAACAGTGCGACCG | |
| | CATTAACAGTCCCTCAATTTGTGGAGGTGGGTAGGTGA | |
| 9 | ATGATGTTATTGGTATGCTTTGATTTACCAAGGGCGACTATCAAGA | E. italicus cas2 |
| | ACCGCAAAGATGCAACAAAATACAGAAAACGTTTAGTTGAGTTGG | |
| | GCTTTACTATGAAACAATTTAGTCTGTATGAGAGAGAAATTCGACA | |
| | GACGGATACTCGTCGAAAAGTGATTGCAACTTTATCGGAGTGCTT | |
| | ACCAGAAGAAGGGCAAATAACATTGTATGAGTTGCCGGATGAAGT | |
| | GAATGATCAACAAATCACGATTTTGGGTGAAAAAGTTGCAAGAAC | |
| | AACTTTTCGAAGAGCCAAGTTTCTAGTATTATAA | |
| 10 | CAACGTAGATAATGTCTCTCATGCTTGAGCTCTCTAGAGGATC | 989RafCasF-GC |
| 11 | GGTAAGAGTTGCAAGAATTATTTTTTTGTAGTTCTCGAGTGCATAT | 989RafCasR-GC |
| | TTTCGG | |
| 12 | TAGGTCAATCTAGCCCATTG | CR-F2 |
| 13 | CGCAGCTTAAAATCCGGCAGG | Sp15R |
| 14 | GCTTGAGCTCTCTAGAGGATC | 989F |
| 15 | TTCTCGAGTGCATATTTTCGG | 989R |
| 16 | CCGAAAATATGCACTCGAGAATTATAATACTAGAAACTTGG | italCas1Cas2F |
| 17 | GATCCTCTAGAGAGCTCAAGCGGGAGATGATAGAAAATGAAGG | italCas1Cas2R |
| 18 | TTCTAATGTCACTAACCTGC | CasSingleF |
| 19 | GCAGGTTAGTGACATTAGAA | CasSingleR |
| 20 | CTTTATTCAGGAGGGAAAATAGTTCTCGAGTGCATATTTTCG | Cas1cloneF |
| 21 | CGAAAATATGCACTCGAGAACTATTTTCCCTCCTGAATAAAG | Cas1cloneR |
| 22 | AAGCTCAGGAGGGAAAATAGATGATGTTGTTGTGTGCTTTTG | Cas2clone-rbsF |
| 23 | CTATTTTCCCTCCTGAGCTTGAGCTCTCTAGAGGATC | Cas2clone-rbsR |

### Results

Lactococcus raffinolactis CRISPR-Cas and Cas1-Cas2 Complementation

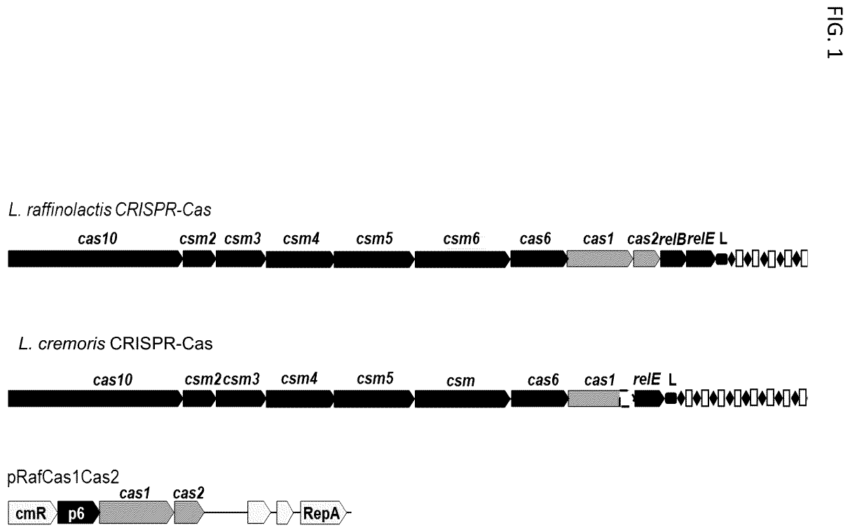

A type III-A CRISPR-Cas related to the L. lactis/L. cremoris CRISPR-Cas was identified in three L. raffinolactis strains. Nucleotide comparison of exemplary L. raffinolactis strain Lr_19_5 CRISPR-Cas to the L. cremoris, formerly referred to as Lactococcus lactis subsp cremoris, p537CR CRISPR-Cas found that the CRISPR repeats are 100% identical, the leader regions are 98% identical, and the trailer regions are 100% identical until its disruption by a mobile element fragment in L. cremoris. The Cas proteins involved in CRISPR interference are highly conserved between p537CR and Lr_19_5 with each protein sharing >97% amino acid identity with the exception of Csm6, which shares 93% amino acid identity (Table E3). Lr_19_5 encodes a cas2 and a relB antitoxin which are absent in L. cremoris. The exemplary L. cremoris cas1 appeared to be truncated compared to the exemplary Lr_19_5 cas1 (FIG. 1).

The exemplary L. raffinolactis cas1-cas2 was cloned behind a p6 promoter in expression vector pTRK989, creating vector pRafCas1Cas2. pTRK989 and pRafCas1Cas2 were transformed into exemplary DGCC12607, resulting in the isolation of 12607-989 and 12607-Cas1Cas2, respectively. A simplified diagram of the exemplary L. cremoris, formerly referred to as Lactococcus lactis subsp cremoris, and the exemplary L. raffinolactis Lr_19_5 CRISPR-Cas regions and pRafCas1Cas2 can be found in FIG. 1.

TABLE E3
Comparison of the exemplary L. cremoris and
L. raffinolactis type III-A CRISPR-Cas locus

| L. cremoris p537CR Protein | |
|---|---|
| (formerly referred to as | % Amino acid ID with |
| Lactococcus lactis subsp. cremoris) | L. raffinolactis Lr_19_5 |
| Cas10 | 99.6 |
| Csm2 | 98.6 |
| Csm3 | 98.6 |
| Csm4 | 99.3 |
| Csm5 | 97.7 |
| Csm6 | 93 |
| Cas6 | 99.5 |
| RelE | 97 |

| L. cremoris | % Nucleotide ID with |
|---|---|
| p537CR Sequence | L. raffinolactis Lr_19_5 |
| Leader | 98 |
| Repeat | 100 |
| Trailer | 100 |

### Spacer Acquisition and Sequence Analysis

Surviving colonies were analyzed following challenges of 12607-989 and 12607-Cas1Cas2 with phage p2.S3.S4.V at two different MOIs (1 and 0.1). The 12607-989 challenge produced 38 colonies at MOI=0.1 and 16 colonies at MOI=1. All colonies were tested for spacer acquisition using PCR with primers CR-F2 and Sp15R (Table E2). Based on amplicon size, no colonies had acquired additional spacers. From the 12607-Cas1Cas2 challenge, 82 and 66 colonies were observed at MOI=0.1 and MOI=1, respectively. From these survivors, 46 (of 82, MOI=0.1) and 47 (of 66, MOI=1) were tested for spacer acquisition using PCR with primers CR-F2 and Sp15R. Based on amplicon size, 28 total colonies acquired additional spacers. Of the isolated colonies that were tested, 6/46 (13%, MOI=0.1) and 22/47 (47%, MOI=1) had acquired new spacers.

CRISPR sequences were analyzed from the 28 colonies with acquired spacers. The six colonies from the MOI=0.1 phage challenge each had one new spacer, four from p2.S3.S4.V, and two from pRafCas1Cas2. All 22 colonies analyzed from the MOI=1 challenge were unique with regard to spacer content, and the number of newly added spacers ranged from one to three. All 22 colonies had acquired a new spacer against p2.S3.S4.V, all of which were located closest to the leader end of the CRISPR array. Typically, adaptive spacer acquisition occurs directionally initiating at the first repeat adjacent to the 3′-end of the leader sequence (McGinn and Marraffini, 2019). Hence, in all isolates the position of the new spacers next to the leader is consistent with the conventional spacer acquisition mechanism. In one case, the same p2.S3.S4.V spacer was acquired by multiple isolates. In colony H5, this p2.S3.S4.V spacer was the only spacer acquired, but in colony F6, this spacer was apparently acquired subsequent to acquisition of a spacer targeting pRafCas1Cas2. Therefore, it is likely that acquisitions of this spacer in colonies F6 and H5 were the result of independent events.

Five isolates from the MOI=1 challenge had acquired new spacers prior to acquisition of spacers targeting p2.S3.S4.V. These spacers were acquired from extrachromosomal elements; lactococcal sex factor pFI430 (resident in the genome of DGCC12067), pRafCas1Cas2, and CRISPR-Cas plasmid p537CR. Three isolates harbored the same two spacers (acquired from pRafCas1Cas2 and pFI430) downstream of their unique p2.S3.S4.V spacers. Additionally, the pRafCas1Cas2 spacer from these three isolates was identical to a spacer acquired by two other isolates, including one from the MOI=0.1 challenge. This suggests that spacer acquisition was occurring in the population before exposure to phage p2.S3.S4.V.

Analysis of protospacer regions found that the spacers acquired from pRafCas1Cas2 and p537CR were oriented such that the spacer RNA transcript is not complimentary to the target mRNA and therefore, will not provide interference, as targeting by type III-A CRISPR-Cas systems is transcription-dependent (Millen et al., 2018, Lactococcus lactis type III-A CRISPR-Cas system cleaves bacteriophage RNA, RNA Biology, 16:4, 461-468). This is consistent with their existence in phage resistant isolates. Effective targeting of pRafCas1Cas2 would prevent acquisition of spacers targeting p2.S3.S4.V, and effective targeting of p537CR would eliminate CRISPR interference, and these cells would not survive the phage challenge. As expected, all spacers acquired from p2.S3.S4.V were found to be oriented to facilitate phage interference.

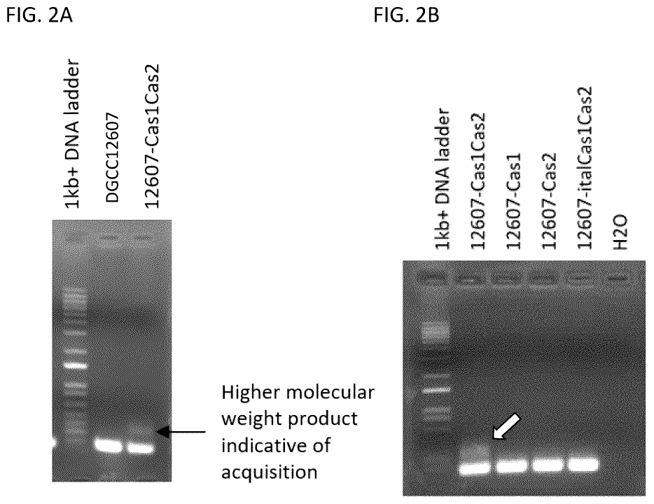

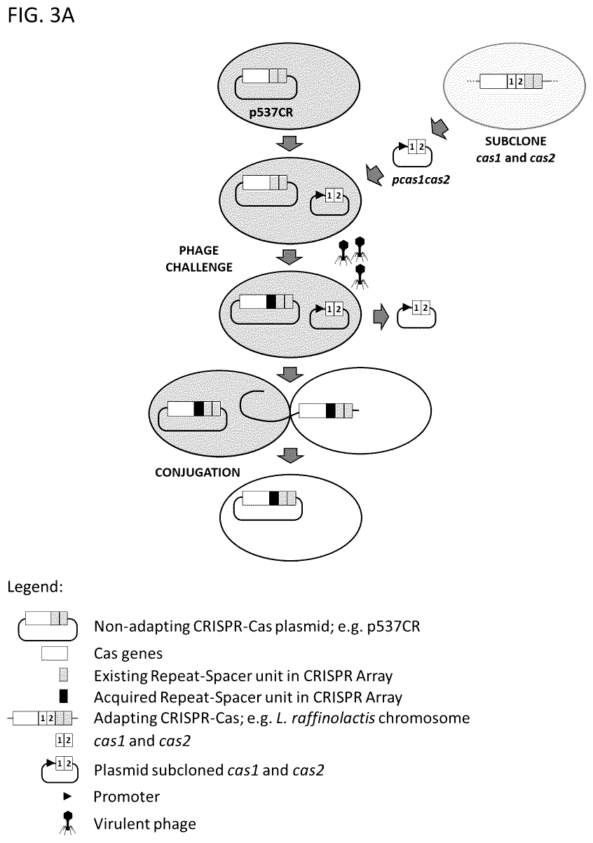

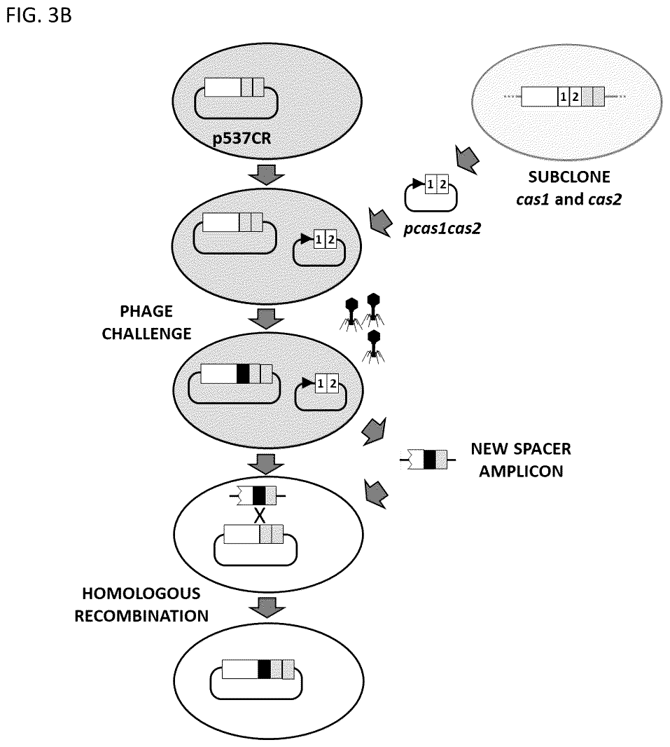

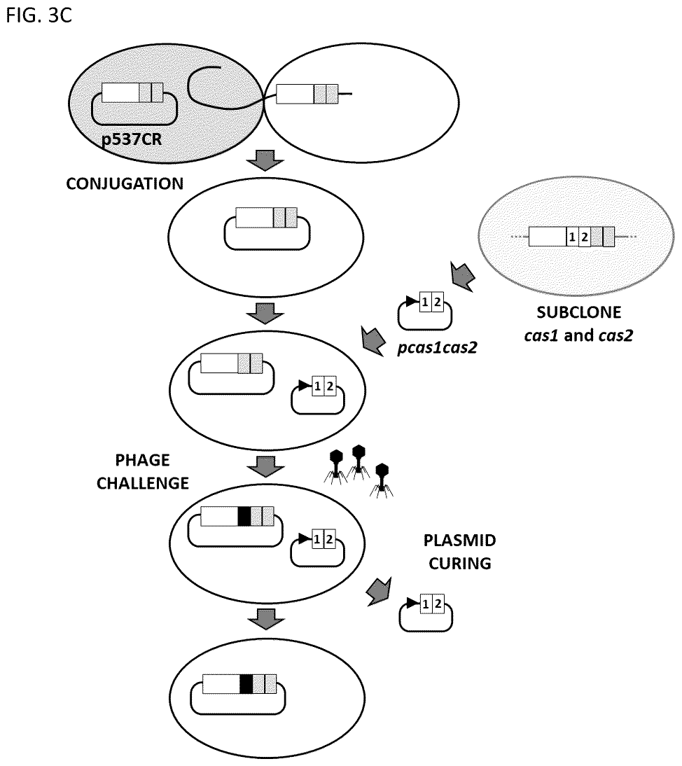

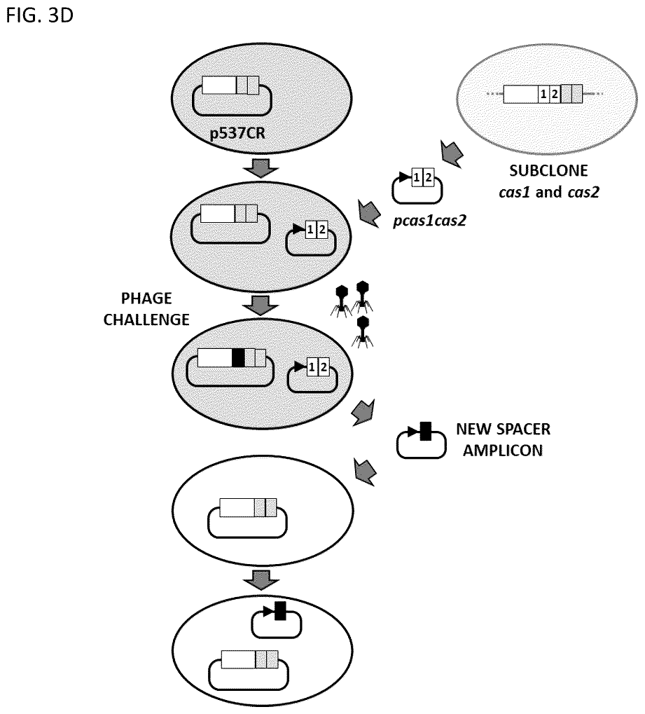

Spacer acquisition was shown to be detectable in a culture population without phage pressure using PCR with primers CR-F2 and Sp15R using overnight culture as a template. A faint higher molecular weight band in addition to the single repeat spacer amplicon was observed in 12607-Cas1Cas2 and interpreted as positive for acquisition in the population (FIG. 2A).

Individual L. raffinolactis Cas1 and Cas2 Complementation and E. Italicus Cas1-Cas2 Complementation To determine if both the exemplary L. raffinolactis cas1 and cas2 were required for the acquisition phenotype rather than cas1 alone or cas2 alone, cas1 and cas2 were cloned individually into pTRK989, creating vectors pRafCas1 and pRafCas2, respectively. Vectors were transformed into DGCC12607, resulting in the isolation of 12607-Cas1 and 12607-Cas2. Spacer acquisition was not observed in 12607-Cas1 or 12607-Cas2 when tested by PCR with primers CR-F2 and Sp15R on overnight culture (FIG. 2B).

To test if a distantly related cas1/cas2 could function in spacer acquisition in L. cremoris, the cas1-cas2 adjacent to a type III-A CRISPR-Cas system in E. italicus DSM15952 was chosen to test in DGCC12607. This specific cas1-cas2 was chosen because there is high conservation over the 3′ half of the L. cremoris and E. italicus CRISPR repeats. This E. italicus Cas1/Cas2 share 46.4/63.4% amino acid identity with L. raffinolactis Cas1/Cas2. E. italicus cas1-cas2 was cloned behind the p6 promoter in pTRK989, creating vector pltalCas1Cas2. pltalCas1Cas2 was transformed into DGCC12607, resulting in the isolation of 12607-italCas1Cas2. Spacer acquisition was not observed in 12607-italCas1Cas2 when tested by PCR with primers CR-F2 and Sp15R on overnight culture (FIG. 2B).

These results are supportive of the ability to enable CRISPR adaptation in an exemplary lactococcal strain using an exemplary L. raffinolactis cas1-cas2 system. The results further suggest that both Cas1 and Cas2 are useful for enabling CRISPR adaptation.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A polynucleotide comprising a nucleic acid sequence encoding a Clustered Regularly Interspaced Short Palindromic Repeats (CRISPR)-associated protein 1 (Cas1) polypeptide.

2. The polynucleotide of claim 1, comprising a nucleic acid sequence encoding a CRISPR-associated endoribonuclease Cas2 (Cas2) polypeptide.

3. A polynucleotide comprising a nucleic acid sequence encoding a CRISPR-associated endoribonuclease Cas2 (Cas2) polypeptide.

4. The polynucleotide of claim 1, wherein the Cas1 polypeptide is a Cas1 polypeptide of a Lactococcus bacterial strain.

5. The polynucleotide of claim 1, wherein the Cas1 polypeptide comprises an amino acid sequence set forth in SEQ ID NO:2 or an amino acid sequence having at least 85% sequence identity to the sequence set forth in SEQ ID NO:2.

6. The polynucleotide of claim 2, wherein the Cas2 polypeptide is a Cas2 polypeptide of a Lactococcus bacterial strain.

7. The polynucleotide of claim 3, wherein the Cas2 polypeptide comprises an amino acid sequence set forth in SEQ ID NO:4 or an amino acid sequence having at least 85% sequence identity to the sequence set forth in SEQ ID NO:4.

8. A vector comprising the polynucleotide of claim 1.

9. A vector comprising the polynucleotide of claim 3.

10. A vector comprising the polynucleotide of claim 2.

11. A bacterial strain comprising a polynucleotide of claim 2.

12. The bacterial strain of claim 11, wherein the bacterial strain comprises a non-adapting CRISPR-Cas system.

13. The bacterial strain of claim 11, wherein the non-adapting CRISPR-Cas system is a type III-A CRISPR-Cas system.

14. The bacterial strain of claim 11, wherein the non-adapting CRISPR-Cas system comprises a nucleic acid sequence encoding the set forth by SEQ ID NO:24 or a sequence having at least 85% sequence identity to the sequence set forth in SEQ ID NO:24.

15. A method of enabling adaptation in a non-adapting CRISPR-Cas system, the method comprising introducing into a bacterial strain comprising a non-adapting CRISPR-Cas system a polynucleotide of claim 2.


SEQUENCE LISTING (25 sequences)

SEQ ID NO: 1 - DNA, 1215 bp, Lactococcus raffinolactis
  source 1..1215: mol_type = genomic DNA
atgagagacattatctacgttgaaaataggtattttatcagtagtcgagaaaatgcgctcagatttcatgactatattaataaagcgacccatttcattgcttttgatgacattgatattttagttttggataatgcaagaagttacttgtcaaatggtgtgattaacgactgtttggatcgaaatattttgattttaacttgcgataataaacattctcctaaagcgattttaagcaatgcttttgctaataaaaaacgcttagaacgcttaagaaatcaattgcaattatcctctaagtcaaagaatcgtctttggcgtaaaattgtgatggcgaaaattaataatcaggctgatgctgttaccttcacggttcaagatggaacagttcatcgagagataattgaactaggaaaaatggtcacagaaggagataaagataatcgcgaagctgtcgttgcacgaaaatattttcggacattatttggtggtaattttaagcgtggtcgttttgatgatgtcatcaattcagcgctcaattatggctatgcacttgtgagggctgtcatcagacgagaattggctatctgtggttttgagatgagctttggtattcatcatatgtcaacagaaaatccttttaatctctctgatgatatgattgaagtttttcggccttttgttgatgtgttggtatttgagataattgtgacgaatgacgtaactgtttttgattatgaaatcaaaaaacttctggttaatatttttctggaaaaatgtgtgatagacggaaaagtcatgtctttaacagatgcagtgcgtgtaactatccagagtttaattacttgtcttgaggatgacagcagtgcacccttgaaacttcctagctttattcaggagggaaaatagatgatgttgttgtgtgcttttgatttgcctcgtgagaccaaagaagaaagaaaagctgcaaataaataccgtaagcgattagtagagcttggatttgcaatgaagcaattttccttgtatgagcgtgaggttcgtcatattgatgttaaaaataggttaattgatattttacgggaagaattaccagatactggagcgattaccctatacctattacccaatgaggtgaatgatgctcagattacgatattgggtgaaaagtcggttaataaaacggtaagagttgcaagaattatttttttgtag

SEQ ID NO: 2 - protein, 302 aa, Lactococcus raffinolactis
MRDIIYVENRYFISSRENALRFHDYINKATHFIAFDDIDILVLDNARSYLSNGVINDCLDRNILILTCDNKHSPKAILSNAFANKKRLERLRNQLQLSSKSKNRLWRKIVMAKINNQADAVTFTVQDGTVHREIIELGKMVTEGDKDNREAVVARKYFRTLFGGNFKRGRFDDVINSALNYGYALVRAVIRRELAICGFEMSFGIHHMSTENPFNLSDDMIEVFRPFVDVLVFEIIVTNDVTVFDYEIKKLLVNIFLEKCVIDGKVMSLTDAVRVTIQSLITCLEDDSSAPLKLPSFIQEGK

SEQ ID NO: 3 - DNA, 144 bp, Lactococcus raffinolactis
  source 1..144: mol_type = genomic DNA
tttctggaaaaatgtgtgatagacggaaaagtcatgtctttaacagatgcagtgcgtgtaactatccagagtttaattacttgtcttgaggatgacagcagtgcacccttgaaacttcctagctttattcaggagggaaaatag

SEQ ID NO: 4 - protein, 101 aa, Lactococcus raffinolactis
MMLLCAFDLPRETKEERKAANKYRKRLVELGFAMKQFSLYEREVRHIDVKNRLIDILREELPDTGAITLYLLPNEVNDAQITILGEKSVNKTVRVARIIFL

SEQ ID NO: 5 - DNA, 306 bp, Lactococcus raffinolactis
  source 1..306: mol_type = genomic DNA
atgatgttgttgtgtgcttttgatttgcctcgtgagaccaaagaagaaagaaaagctgcaaataaataccgtaagcgattagtagagcttggatttgcaatgaagcaattttccttgtatgagcgtgaggttcgtcatattgatgttaaaaataggttaattgatattttacgggaagaattaccagatactggagcgattaccctatacctattacccaatgaggtgaatgatgctcagattacgatattgggtgaaaagtcggttaataaaacggtaagagttgcaagaattatttttttgtag

SEQ ID NO: 6 - protein, 302 aa, Enterococcus italicus
MKDIIYVERKYFLTVKGESIKFVNVMDKTEKYIPLDDVEWLIFDHPSSYFSNKLITECMVRGIGVLFCDGKHSPDAILLNQFGHRQRLTRVNHQLSVSNRTKKRLWKKIIRTKIHNQAQCIDQLTDNQKSRDYLMSLSNSVQEGDSSNREAVAAKIYFTALFGENFRRGRYDDVVNSGLNYGYALVRGMIKKELARYGVEQSFGIHHKSSENPFNLADDIIEPFRPFVDRSVYENFYLTEKTTFEHTDKQALFQVFFDSCIIQGKVFQLTDAINQAVQSFVKSIDQNSATALTVPQFVEVGR

SEQ ID NO: 7 - DNA, 909 bp, Enterococcus italicus
  source 1..909: mol_type = genomic DNA
atgaaggatattatttatgttgaacgaaagtattttttaaccgtaaaaggggagtccataaaattcgtaaacgtgatggacaaaacggaaaaatacatcccgcttgatgatgtggaatggttgatttttgatcatcctagtagttatttttcaaacaaattaattactgaatgtatggttagagggataggtgtgcttttttgcgatggcaagcattcaccagatgctattttactgaatcagtttggacatcgacaaagattaacgcgagtgaatcatcaattatctgtatcgaatcgaacaaaaaaacgattatggaagaaaataatcaggaccaaaattcataatcaagcccaatgcattgatcaattaacagataaccagaagagcagagattaccttatgtctctgtcaaattctgtgcaggaaggagacagttcaaatagagaggcggtggcagctaagatttacttcactgcattatttggtgaaaactttaggcgcggacgctatgatgatgttgtaaatagtggcttaaattatgggtatgcacttgttagaggaatgatcaaaaaagaactggcaaggtatggtgtagaacagagctttggcatccatcataaatcaagtgaaaatccgtttaatttagctgatgacattattgaaccttttcgaccattcgtagatcgaagtgtgtatgaaaatttttatttgacagaaaaaacgactttcgaacatactgacaaacaagcactatttcaagttttttttgattcatgcatcattcaaggaaaagtgtttcaactgacggatgccattaatcaagcagtgcaatctttcgtaaaatcaattgatcagaacagtgcgaccgcattaacagtccctcaatttgtggaggtgggtaggtga

SEQ ID NO: 8 - protein, 101 aa, Enterococcus italicus
MMLLVCFDLPRATIKNRKDATKYRKRLVELGFTMKQFSLYEREIRQTDTRRKVIATLSECLPEEGQITLYELPDEVNDQQITILGEKVARTTFRRAKFLVL

SEQ ID NO: 9 - DNA, 306 bp, Enterococcus italicus
  source 1..306: mol_type = genomic DNA
atgatgttattggtatgctttgatttaccaagggcgactatcaagaaccgcaaagatgcaacaaaatacagaaaacgtttagttgagttgggctttactatgaaacaatttagtctgtatgagagagaaattcgacagacggatactcgtcgaaaagtgattgcaactttatcggagtgcttaccagaagaagggcaaataacattgtatgagttgccggatgaagtgaatgatcaacaaatcacgattttgggtgaaaaagttgcaagaacaacttttcgaagagccaagtttctagtattataa

SEQ ID NO: 10 - DNA, 43 bp, synthetic construct
  misc_feature 1..43: note = 989RafCasF-GC
  source 1..43: mol_type = other DNA
caacgtagataatgtctctcatgcttgagctctctagaggatc

SEQ ID NO: 11 - DNA, 52 bp, synthetic construct
  misc_feature 1..52: note = 989RafCasR-GC
  source 1..52: mol_type = other DNA
ggtaagagttgcaagaattatttttttgtagttctcgagtgcatattttcgg

SEQ ID NO: 12 - DNA, 20 bp, synthetic construct
  misc_feature 1..20: note = CR-F2
  source 1..20: mol_type = other DNA
taggtcaatctagcccattg

SEQ ID NO: 13 - DNA, 21 bp, synthetic construct
  misc_feature 1..21: note = Sp15R
  source 1..21: mol_type = other DNA
cgcagcttaaaatccggcagg

SEQ ID NO: 14 - DNA, 21 bp, synthetic construct
  misc_feature 1..21: note = 989F
  source 1..21: mol_type = other DNA
gcttgagctctctagaggatc

SEQ ID NO: 15 - DNA, 21 bp, synthetic construct
  misc_feature 1..21: note = 989R
  source 1..21: mol_type = other DNA
ttctcgagtgcatattttcgg

SEQ ID NO: 16 - DNA, 41 bp, synthetic construct
  misc_feature 1..41: note = italCas1Cas2F
  source 1..41: mol_type = other DNA
ccgaaaatatgcactcgagaattataatactagaaacttgg

SEQ ID NO: 17 - DNA, 43 bp, synthetic construct
  misc_feature 1..43: note = italCas1Cas2R
  source 1..43: mol_type = other DNA
gatcctctagagagctcaagcgggagatgatagaaaatgaagg

SEQ ID NO: 18 - DNA, 20 bp, synthetic construct
  misc_feature 1..20: note = CasSingleF
  source 1..20: mol_type = other DNA
ttctaatgtcactaacctgc

SEQ ID NO: 19 - DNA, 20 bp, synthetic construct
  misc_feature 1..20: note = CasSingleR
  source 1..20: mol_type = other DNA
gcaggttagtgacattagaa

SEQ ID NO: 20 - DNA, 42 bp, synthetic construct
  misc_feature 1..42: note = Cas1cloneF
  source 1..42: mol_type = other DNA
ctttattcaggagggaaaatagttctcgagtgcatattttcg

SEQ ID NO: 21 - DNA, 42 bp, synthetic construct
  misc_feature 1..42: note = Cas1cloneR
  source 1..42: mol_type = other DNA
cgaaaatatgcactcgagaactattttccctcctgaataaag

SEQ ID NO: 22 - DNA, 42 bp, synthetic construct
  misc_feature 1..42: note = Cas2clone-rbsF
  source 1..42: mol_type = other DNA
aagctcaggagggaaaatagatgatgttgttgtgtgcttttg

SEQ ID NO: 23 - DNA, 37 bp, synthetic construct
  misc_feature 1..37: note = Cas2clone-rbsR
  source 1..37: mol_type = other DNA
ctattttccctcctgagcttgagctctctagaggatc

SEQ ID NO: 24 - protein, 251 aa, Lactococcus cremoris
MRDIIYVENRYFISSRENALRFHDYINKATHFIAFDDIDILVLDNARSYLSNGVINDCLDRNILILTCDNKHSPKAILSNAFANKKRLERLRNQLQLSSKSKNRLWRKIVMAKINNQADAVTFTVQDGTVHREIIELGKMVTEGDKDNREAVVARKYFRTLFGGNFKRGRFDDVINSALNYGYALVRAVIRRELAICGFEMSFGIHHMSTENPFNLSDDMIEVFRPFVDVLVFEIIVTNYKRKFKRAIKVF

SEQ ID NO: 25 - DNA, 756 bp, Lactococcus cremoris
  source 1..756: mol_type = genomic DNA
atgagagacattatctacgttgaaaataggtattttatcagtagtcgagaaaatgcgctcagatttcatgactatattaataaagcgacccatttcattgcttttgatgacattgatattttagttttggataatgcaagaagttacttgtcaaatggtgtgattaacgactgtttggatcgaaatattttgattttaacttgcgataataaacattctcctaaagcgattttaagcaatgcttttgctaataaaaaacgcttagaacgcttaagaaatcaattgcaattatcctctaagtcaaagaatcgtctttggcgtaaaattgtgatggcgaaaattaataatcaggctgatgctgttaccttcacggttcaagatggaacagttcatcgagagataattgaactaggaaaaatggtcacagaaggagataaagataatcgcgaagctgtcgttgcacgaaaatattttcggacattatttggtggtaattttaagcgtggtcgttttgatgatgtcatcaattcagcgctcaattatggctatgcacttgtgagggctgtcatcagacgagaattggctatctgtggttttgagatgagctttggtattcatcatatgtcaacagaaaatccttttaatctctctgatgatatgattgaagtttttcggccttttgttgatgtgttggtatttgagataattgtgacgaactacaagcggaagttcaaaagggctatcaaagttttttaa